(19) United States
(12) Patent Application Publication
(10) Pub. No.: US 2023/0128972 A1
(43) Pub. Date: Apr. 27, 2023

(54) Anat Inhibitors and Methods of Use Thereof

(71) Applicants: Atomwise Inc., San Francisco, CA (US); The University of Toledo, Toledo, OH (US)

(72) Inventors: Ronald E. Viola, Toledo, OH (US); Adrian Stecula, San Francisco, CA (US)

(21) Appl. No.: 17/794,911

(22) PCT Filed: Jan. 29, 2021
(86) PCT No.: PCT/US2021/015862

§ 371 Date: Jul. 22, 2022

Related U.S. Application Data
(60) Provisional application No. 62/968,839, filed on Jan. 31, 2020.

(51) Int. Cl. A61K 31/498; A61K 31/505; A61K 31/433; A61K 31/4365; A61K 31/4245

(52) U.S. Cl. CPC A61K 31/498; A61K 31/505; A61K 31/433; A61K 31/4365; A61K 31/4245

(57) ABSTRACT

Inhibitors of a critical brain enzyme, N-acetyltransferase (ANAT), and methods of discovering, making and using the same for the treatment of disease are disclosed.

### CROSS REFERENCE TO RELATED APPLICATIONS

This application claims the benefit of U.S. Provisional Patent Application No. 62/968,839, filed on Jan. 31, 2020, which is incorporated by reference in its entirety.

### FIELD

The disclosure relates generally to compounds and methods of using the same for treating conditions characterized by elevated levels of the brain metabolite N-acetylaspartate (NAA), and/or by defects in the enzyme responsible for the metabolism of NAA, including compounds and methods of using the same that bind aspartate N-acetyltransferase (ANAT).

### BACKGROUND

Over 8,000 diseases are classified as rare, and cumulatively affect approximately 350 million people worldwide. This large and diverse set of diseases represents a plurality of urgent unmet medical needs each requiring specific examination. Therefore, methods to accelerate drug discovery and development—without incurring the unsustainable costs of simply scaling typical discovery processes in parallel—are of paramount importance. One burgeoning approach to identify new starting points in the drug discovery process is machine learning.

Machine learning is a subfield of statistics concerned with computationally-expensive predictive algorithms fit to large datasets, and has yielded super-human performance in fields ranging from game playing (for example, chess, go, and real-time strategy games) to speech processing, and diagnosing diabetic retinopathy. In chemistry, machine learning approaches promise to identify useful and safe molecules with high accuracy, and inform the deployment of always-limited synthetic resources. Computational approaches are unique in that they decouple the potential scope of screening from the cost and time-limiting physical challenges of expressing and purifying proteins; synthesizing, characterizing and storing molecules; culturing and maintaining cell lines; and running in vitro assays with combinations of the components above to generate the desired data outputs. Predictive algorithms permit in-silico evaluation of modern synthesize-on-demand libraries, which number in the billions of diverse molecules and avoid the narrow scaffold diversity that has historically been a problem of combinatorial or DNA-encoded libraries.

### SUMMARY

In some embodiments, the disclosure provides a compound of any one of Formulas I to VIII, or a pharmaceutically acceptable analog, derivative, salt, solvate, hydrate, cocrystal, or prodrug thereof, wherein in some embodiments, the compound is an ANAT inhibitor, wherein in Formulas I to VIII: U, V, W, V′, W′, Y′, X, Y, and Z are independently selected at each occurrence from C, CH, CH₂, N, NH, NR^{a}, O, S; G is independently at each occurrence a mono- or polycyclic optionally substituted cycloalkyl, mono- or polycyclic optionally substituted heterocycloalkyl, mono- or polycyclic optionally substituted aryl, mono- or polycyclic optionally substituted arylalkyl, mono- or polycyclic optionally substituted heteroaryl, and mono- or polycyclic optionally substituted heteroarylalkyl; R₁ R₂, and R₃ are independently selected at each occurrence from hydrogen, optionally substituted alkylheteroaryl, optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted alkylheteroaryl, optionally substituted heteroarylalkyl, optionally substituted alkoxy, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂, wherein R₁, R₂, and R₃ can optionally be joined to form a carbo- or heterocycle; L₁, L₂, and L₃ are linkers comprising independently at each occurrence one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a}C(O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl; wherein any optional substituent is independently selected at each occurrence from optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂; R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and a, b, c, d, e, f, g, h, and i are independently at each occurrence an integer selected from 0, 1, 2, 3, 4, 5, and 6. In some embodiments, G is independently at each occurrence selected from benzene, pyridine, pyrimidine, pyrazine, pyridazine, and triazine. In some embodiments, G is independently at each occurrence selected from furan, thiophene, pyrrole, thiazole, isothiazole, 1,2,3-thiadiazole, imidazole, oxazole, isoxazole, 1,2,3-oxadiazole, 1,3,4-oxadiazole, 1,2,4-oxadiazole, 1,2,3-triazole, 1,2,4-triazole, and pyrazole. In some embodiments, G is independently at each occurrence selected from naphthalene, quinoline, isoquinoline, cinnoline, quinoxaline, phtalazine, pyridopyrazine, pteridine, pyridopyridazine, naphtyridine, carbazole, dibenzofuran, or quinazoline. In some embodiments, G is independently at each occurrence selected from indole, benzoxazole, benzothiophene, benzimidazole, indazole, benzotriazole, pyrrolopyridine, triazolopyridine, purine, indolizine, pyrrolopyrimidine, pyrrolopyrazine, pyrrolopyrimine, pyrrolopyridazine, imidazopyridine, pyrazolopyridine, imidazopyridazine, or imidazopyrimidine.

In some embodiments, the disclosure provides a compound of any one of Formulas 1 to 5, or a pharmaceutically acceptable analog, derivative, salt, solvate, hydrate, cocrystal, or prodrug thereof, wherein in some embodiments, the compound is an ANAT inhibitor.

In some embodiments, the disclosure provides a compound of any one of Formulas 1001 to 1024, or a pharmaceutically acceptable analog, derivative, salt, solvate, hydrate, cocrystal, or prodrug thereof, wherein in some embodiments, the compound is an ANAT inhibitor.

In some embodiments, the disclosure provides a compound of any one of Formulas 2001 to 2025, or a pharmaceutically acceptable analog, derivative, salt, solvate, hydrate, cocrystal, or prodrug thereof, wherein in some embodiments, the compound is an ANAT inhibitor.

In some embodiments, the disclosure provides a compound of any one of Formulas 3001 to 3023, or a pharmaceutically acceptable analog, derivative, salt, solvate, hydrate, cocrystal, or prodrug thereof, wherein in some embodiments, the compound is an ANAT inhibitor.

In some embodiments, the disclosure provides a compound of any one of Formulas 4001 to 4030, or a pharmaceutically acceptable analog, derivative, salt, solvate, hydrate, cocrystal, or prodrug thereof, wherein in some embodiments, the compound is an ANAT inhibitor.

In some embodiments, the disclosure provides a compound of any one of Formulas 5001 to 5023, or a pharmaceutically acceptable analog, derivative, salt, solvate, hydrate, cocrystal, or prodrug thereof, wherein in some embodiments, the compound is an ANAT inhibitor.

In some embodiments, the disclosure provides a compound of any one of Formulas 6001 to 6005, or a pharmaceutically acceptable analog, derivative, salt, solvate, hydrate, cocrystal, or prodrug thereof, wherein in some embodiments, the compound is an ANAT inhibitor.

In some embodiments, the disclosure provides a pharmaceutical composition comprising a therapeutically effective amount of an ANAT inhibitor of any one of Formulas I to VIII, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, or a pharmaceutically acceptable analog, derivative, salt, solvate, hydrate, cocrystal, or prodrug thereof. In some embodiments, the disease is Canavan disease. In some embodiments, the disease is cancer. In some embodiments, the disease is lung cancer. In some embodiments, the disease is an inborn error of metabolism. In some embodiments, the disease is epilepsy. In some embodiments, the disease is leukodystrophy. In some embodiments, the disease is N-acetyl aspartate deficiency. In some embodiments, the subject is human. In some embodiments, the ANAT inhibitor is administered in a dosage unit form. In some embodiments, the dosage unit comprises a physiologically compatible carrier medium.

In some embodiments, the disclosure provides methods of treating or preventing diseases alleviated by inhibiting ANAT in a subject in need thereof, comprising administering to the patient a therapeutically effective amount of an ANAT inhibitor of any one of Formulas I to VIII, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, or a pharmaceutically acceptable analog, derivative, salt, solvate, hydrate, cocrystal, or prodrug thereof. In some embodiments, the disease is Canavan disease. In some embodiments, the disease is cancer. In some embodiments, the disease is lung cancer. In some embodiments, the disease is an inborn error of metabolism. In some embodiments, the disease is epilepsy. In some embodiments, the disease is leukodystrophy. In some embodiments, the disease is N-acetyl aspartate deficiency. In some embodiments, the subject is human. In some embodiments, the ANAT inhibitor is administered in a dosage unit form. In some embodiments, the dosage unit comprises a physiologically compatible carrier medium.

In some embodiments, the disclosure provides method of treating or preventing disease alleviated by inhibiting ANAT in a subject in need thereof, comprising administering to the patient a therapeutically effective amount of a pharmaceutical composition comprising a therapeutically effective amount of an ANAT inhibitor of any one of Formulas I to VIII, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, or a pharmaceutically acceptable analog, derivative, salt, solvate, hydrate, cocrystal, or prodrug thereof. In some embodiments, the disease is Canavan disease. In some embodiments, the disease is cancer. In some embodiments, the disease is lung cancer. In some embodiments, the disease is an inborn error of metabolism. In some embodiments, the disease is epilepsy. In some embodiments, the disease is leukodystrophy. In some embodiments, the disease is N-acetyl aspartate deficiency. In some embodiments, the subject is human. In some embodiments, the ANAT inhibitor is administered in a dosage unit form. In some embodiments, the dosage unit comprises a physiologically compatible carrier medium.

### BRIEF DESCRIPTION OF THE FIGURES

FIGS. 1A and 1B illustrate the reactions catalyzed by three proteins of interest, in accordance with some embodiments of the present disclosure. FIG. 1A illustrates the reactions catalyzed by the enzymes aspartate N-acetyltransferase (ANAT) and aspartoacylase (ASPA). FIG. 1B illustrates the reaction catalyzed by the ANAT homologue Streptomyces hygroscopicus phosphinothricin N-acetyltransferase (PDB ID 5T7E, 20% sequence identity).

FIG. 2 illustrates a cartoon representation of the Streptomyces hygroscopicus phosphinothricin N-acetyltransferase (PDB ID 5T7E, white) and the comparative structure model of the human N-acetylaspartate synthetase (aqua), in accordance with some embodiments of the present disclosure. Acetyl CoA is shown in white as sticks (overlaid from PDB ID 5T7D). Phosphinothricin is shown in grey as sticks.

FIG. 3 illustrates a cartoon representation of the comparative structure model of the human N-acetylaspartate synthetase (aqua), in accordance with some embodiments of the present disclosure. Acetyl CoA is shown in white as sticks (overlaid from PDB ID 5T7D). Residues outlining the predicted aspartic acid binding site are shown as sticks.

FIG. 4 illustrates a surface representation of the comparative structure model of the human N-acetylaspartate synthetase (aqua), in accordance with some embodiments of the present disclosure. Acetyl CoA is shown in white as sticks (overlaid from PDB ID 5T7D). Residues outlining the predicted aspartic acid binding site are shown in white.

FIGS. 5A-5E illustrate the dose-response protocols of the compounds of (A) Formula 1, (B) Formula 2, (C) Formula 3, (D) Formula 4, and (E) Formula 5 with low-micromolar potency against ANAT, in accordance with some embodiments of the present disclosure.

FIGS. 6A-6AG illustrate additional ANAT inhibitor compounds screened and tested.

### DETAILED DESCRIPTION

Rare neglected diseases may be neglected, but are hardly rare, affecting hundreds of millions of people around the world. A hit identification approach using AtomNet, the world's first deep convolutional neural network for structure-based drug discovery, is described herein to identify inhibitors targeting aspartate N-acetyltransferase (ANAT), a promising target for the treatment of patients suffering from Canavan disease. Despite the lack of a protein structure and high sequence identity homologs, the approach successfully identified five low-micromolar inhibitors with drug-like properties.

Unless defined otherwise, all technical and scientific terms used herein have the same meaning as is commonly understood by one of skill in the art to which this disclosure belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include the compounds described herein and, more specifically: an ANAT inhibitor of any one of Formulas I to VIII, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and any pharmaceutically acceptable analogs, derivatives, salts, solvates, hydrates, cocrystals, or prodrugs thereof. The terms “active pharmaceutical ingredient” and “drug” may also include those compounds described herein and any pharmaceutically acceptable analogs, derivatives, salts, solvates, hydrates, cocrystals, or prodrugs thereof that bind aspartate N-acetyltransferase (ANAT).

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells. The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs disclosed herein, can also be incorporated into the described compositions and methods.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule. In some embodiments, the biological molecules modulated by the methods and compounds of the disclosure to effect treatment may include aspartate N-acetyltransferase (ANAT).

As used herein, the term “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of an ANAT inhibitor of any one of Formulas I to VIII, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005. The amino acid residues include but are not limited to the 20 naturally occurring amino acids commonly designated by one or three letter symbols but also include, for example, 4-hydroxyproline, hydroxylysine, desmosine, isodesmosine, 3- methylhistidine, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters (e.g., methyl esters and acetoxy methyl esters). Prodrug esters as employed herein includes esters and carbonates formed by reacting one or more hydroxyls of compounds of the method of the disclosure with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent, e.g., an ANAT inhibitor of any one of Formulas I to VIII, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and any pharmaceutically acceptable analogs, derivatives, salts, solvates, hydrates, or cocrystals thereof, is a prodrug within the scope of the disclosure. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the disclosure.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, or from 0% to 10%, or from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocyclyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acylsulfonamide” refers a —S(O)₂—N(R^{a})—C(═O)— radical, where R^{a} is hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Unless stated otherwise specifically in the specification, an acylsulfonamide group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{a}, and —NR^{a}R^{a} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “aryloxy” refers to the group —O-aryl.

The term “substituted aryloxy” refers to aryloxy wherein the aryl substituent is substituted (i.e., —O-(substituted aryl)). Unless stated otherwise specifically in the specification, the aryl moiety of an aryloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” or “Het” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

For the avoidance of doubt, it is intended herein that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All embodiments of the invention can, in the alternative, be more specifically defined by any of the transitional terms “comprising,” “consisting essentially of,” and “consisting of.”

### Aspartate N-acetyltransferase (ANAT) Inhibitors

The disclosure provides compounds that are inhibitors of aspartate N-acetyltransferase (ANAT). Computational chemistry is both old and new. Numerical modeling has been applied to chemistry as early as the Hammett studies of the reaction rates of substituted benzoic acid eighty years ago. The size and scope of freely-available chemical data today is unprecedented (for example, PubChem at present lists over 250 million bioactivities) and is driving a proliferation of new statistical methods for diverse molecular prediction tasks. Current machine learning methods for identifying small molecule binders to protein targets include a diversity of ligand-based and structure-based approaches. For a broad survey of machine learning methods for hit identification, see, e.g., Carpenter et al.

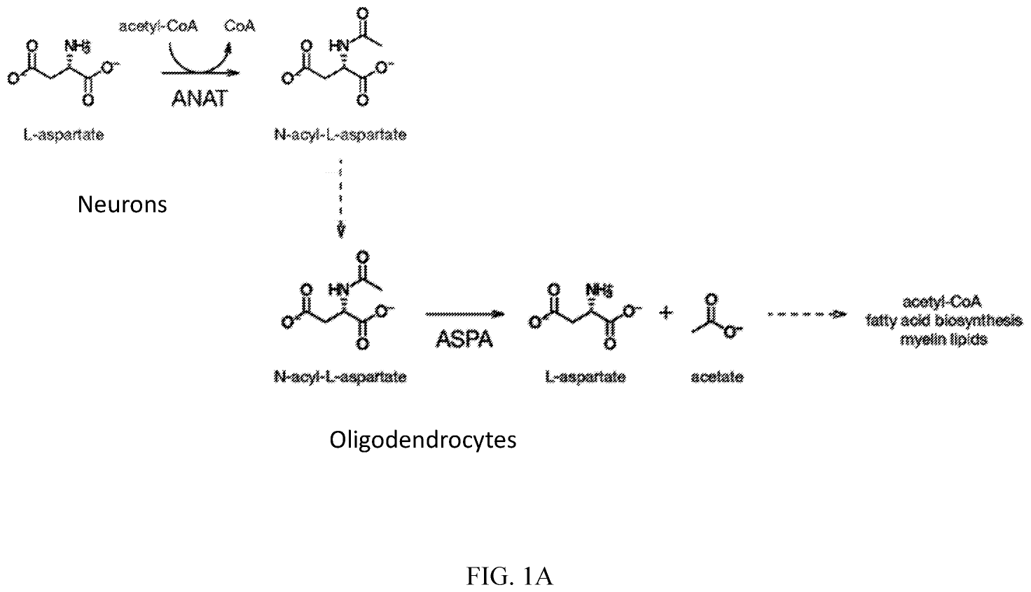

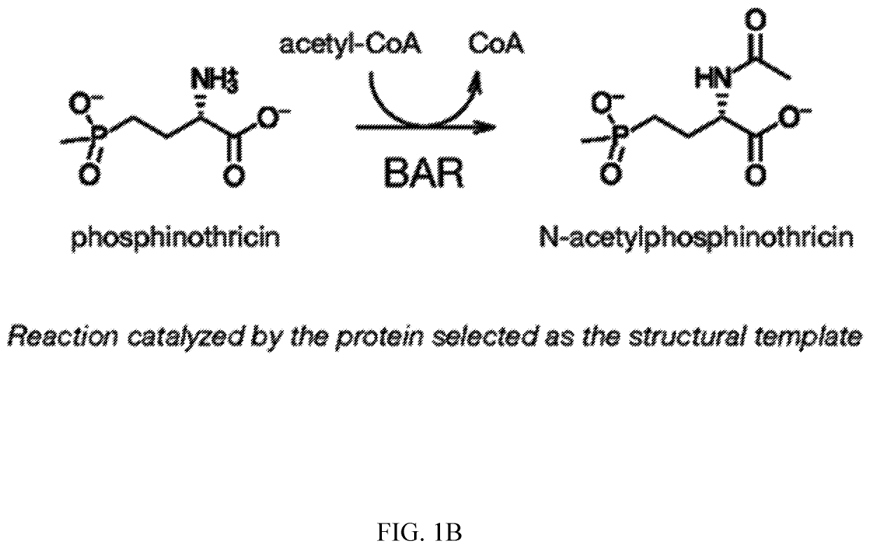

Canavan disease (CD) is a rare disease affecting several hundred people world-wide, with an estimated carrier rate as high as 1 in 40 among the Ashkenazi Jewish population. It is caused by mutations in the aspA gene that codes for the enzyme aspartoacylase (ASPA). These mutations result in amino acid substitutions that produce mutant enzyme isoforms which are expressed, but are either unstable or have diminished catalytic activity. Failure to produce an optimally functional form of this enzyme leads to the accumulation of the critical brain metabolite N-acetyl-L-aspartate (NAA), and a subsequent deficiency in acetate production in oligodendrocyte cell. This, in turn, has been proposed to lead to decreased fatty acid biosynthesis and neuronal demyelination (FIG. 1A). Despite the identification of the specific defects in this gene, the availability of a disease biomarker, and the use of routine genetic screening in high impact populations, new cases of CD with unique mutations continue to emerge, while CD remains a fatal and incurable disease due to the absence of any effective treatments. The devastating symptoms of CD—blindness, lack of motor skills, and dramatically shortened lifetimes—demand the pursuit of additional therapeutic avenues to identify new treatment options.

The vast majority of research conducted over the past several decades to develop treatments for CD have focused on ASPA, the defective enzyme that is exclusively responsible for the metabolism of NAA into aspartic acid and acetate in the brain (FIG. 1A). Several studies have examined the role of elevated NAA in the etiology of CD, hypothesizing that the accumulation of NAA to toxic levels is either the direct cause of CD or indirectly leads to the symptoms through alterations in the osmotic balance in these cells.

The synthesis of NAA in the brain is catalyzed by the enzyme aspartate N-acetyltransferase (ANAT), encoded by the nat8l gene (FIG. 1A). A knockout of the nat8l gene resulted in the elimination of the brain defects that are the hallmark of CD. Immunohistology and TEM imaging showed no evidence of the extensive vacuolation and demyelination found in CD, and the lowering of NAA levels decreased the loss of neurons seen in the cortex and cerebellum regions of the brain. This elimination of developmental defects occurred despite the diminished ability of these animals to metabolize NAA to aspartate and acetate, which is the metabolic consequence which has been the focus of most studies for the treatment of CD. Not only are these developmental defects eliminated by lowering NAA levels, but behavioral and performance evaluations indicate dramatic improvements compared to the impaired functions of the Canavan mouse model.

A critical barrier preventing further examination of the effect of decreased NAA levels in CD patients has been the unavailability of ANAT and a lack of tool compounds for the interrogation of the pathway. ANAT is a membrane-associated enzyme, which poses challenges for expression and purification. Nevertheless, recent advances have finally succeeded in designing, expressing, and purifying a soluble ANAT construct.

Despite these advancements in the etiology of CD, there are no FDA-approved ANAT inhibitors on the market today. The current set of known inhibitors was identified through a fragment library screen, which was subsequently iteratively optimized to produce the first potent inhibitors of this enzyme.

The present disclosure describes a novel approach for hit identification of ANAT. Considering a lack of publicly available structures, as well as a general lack of templates with high sequence identify, the approach comprises generating a comparative structure model based on a template structure which falls well within the “twilight zone” of protein sequence homology. The approach further comprises using AtomNet, a deep convolutional neural network, to screen a library of several million readily available commercial compounds. The approach further comprises testing high-confidence predicted binders in vitro to prospectively discover the first set of drug-like small molecule inhibitors of ANAT.

In some embodiments, the compounds described herein may decrease the activity of ANAT. In some embodiments, the compounds described herein may inhibit the activity of ANAT. In some embodiments, the compounds described herein may modulate the activity of ANAT. In some embodiments, the compounds described herein may be delivered as a listed or as a pharmaceutically acceptable salt, solvate, hydrate, cocrystal, tautomer, or prodrug thereof.

The disclosure provides in one aspect a compound of any one of Formulas I to VIII described herein, or a pharmaceutically acceptable analog, derivative, salt, solvate, hydrate, cocrystal, or prodrug thereof, wherein in some embodiments, the compound is an ANAT inhibitor, wherein in Formulas I to VIII: U, V, W, V′, W′, Y′, X, Y, and Z are independently selected at each occurrence from C, CH, CH₂, N, NH, NR^{a}, O, S; G is independently at each occurrence a mono- or polycyclic optionally substituted cycloalkyl, mono- or polycyclic optionally substituted heterocycloalkyl, mono- or polycyclic optionally substituted aryl, mono- or polycyclic optionally substituted arylalkyl, mono- or polycyclic optionally substituted heteroaryl, and mono- or polycyclic optionally substituted heteroarylalkyl; R₁, R₂, and R₃ are independently selected at each occurrence from hydrogen, optionally substituted alkylheteroaryl, optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted alkylheteroaryl, optionally substituted heteroarylalkyl, optionally substituted alkoxy, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂, wherein R₁, R₂, and R₃ can optionally be joined to form a carbo- or heterocycle; L₁, L₂, and L₃ are linkers comprising independently at each occurrence one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a}C(O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl; wherein any optional substituent is independently selected at each occurrence from optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂; R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and a, b, c, d, e, f, g, h, and i are independently at each occurrence an integer selected from 0, 1, 2, 3, 4, 5, and 6. In some embodiments, G is independently at each occurrence selected from benzene, pyridine, pyrimidine, pyrazine, pyridazine, and triazine. In some embodiments, G is independently at each occurrence selected from furan, thiophene, pyrrole, thiazole, isothiazole, 1,2,3-thiadiazole, imidazole, oxazole, isoxazole, 1,2,3-oxadiazole, 1,3,4-oxadiazole, 1,2,4-oxadiazole, 1,2,3-triazole, 1,2,4-triazole, and pyrazole. In some embodiments, G is independently at each occurrence selected from naphthalene, quinoline, isoquinoline, cinnoline, quinoxaline, phtalazine, pyridopyrazine, pteridine, pyridopyridazine, naphtyridine, carbazole, dibenzofuran, or quinazoline. In some embodiments, G is independently at each occurrence selected from indole, benzoxazole, benzothiophene, benzimidazole, indazole, benzotriazole, pyrrolopyridine, triazolopyridine, purine, indolizine, pyrrolopyrimidine, pyrrolopyrazine, pyrrolopyrimine, pyrrolopyridazine, imidazopyridine, pyrazolopyridine, imidazopyridazine, or imidazopyrimidine.

The disclosure also provides a compound of any one of Formulas 1 to 5, or a pharmaceutically acceptable analog, derivative, salt, solvate, hydrate, cocrystal, or prodrug thereof, wherein in some embodiments, the compound is an ANAT inhibitor:

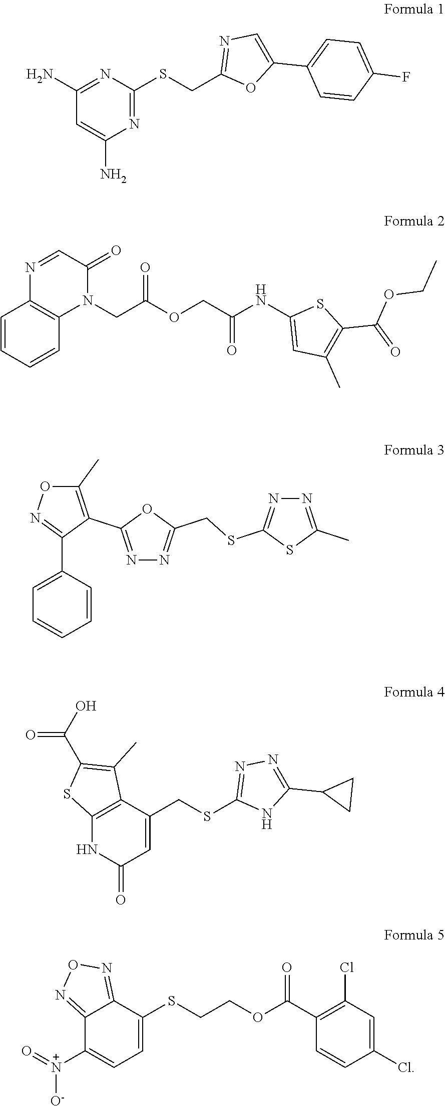 [SMILES: Nc1cc(N)nc(SCc2ncc(-c3ccc(F)cc3)o2)n1 ; CCOC(=O)c1sc(NC(=O)COC(=O)Cn2c(=O)cnc3ccccc32)cc1C ; Cc1nnc(SCc2nnc(-c3c(-c4ccccc4)noc3C)o2)s1 ; Cc1c(C(=O)O)sc2[nH]c(=O)cc(CSc3nnc(C4CC4)[nH]3)c12 ; O=C(OCCSc1ccc([N+](=O)[O-])c2nonc12)c1ccc(Cl)cc1Cl]

The disclosure also provides a compound of any one of Formulas 1001 to 1024, or a pharmaceutically acceptable analog, derivative, salt, solvate, hydrate, cocrystal, or prodrug thereof, wherein in some embodiments, the compound is an ANAT inhibitor:

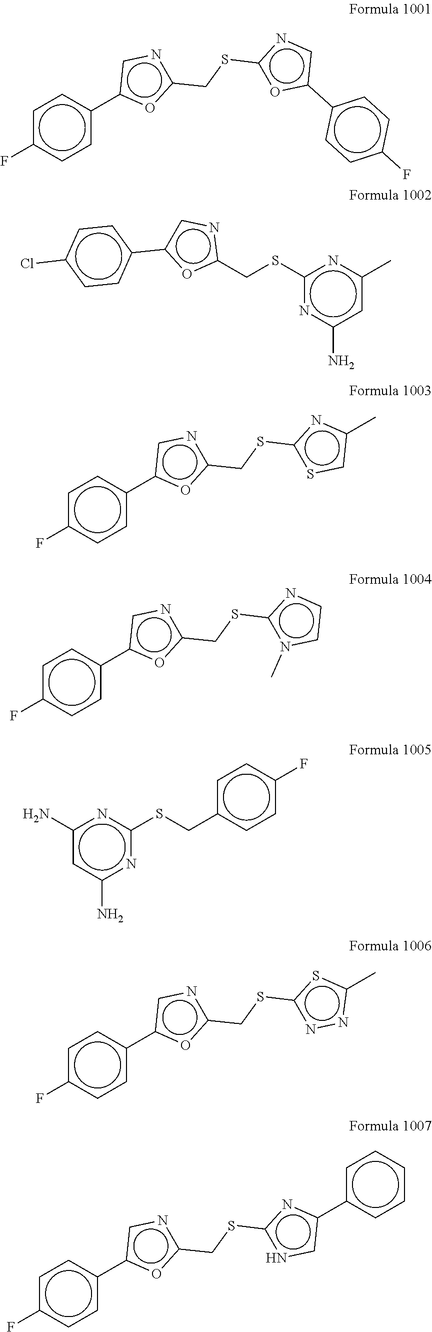

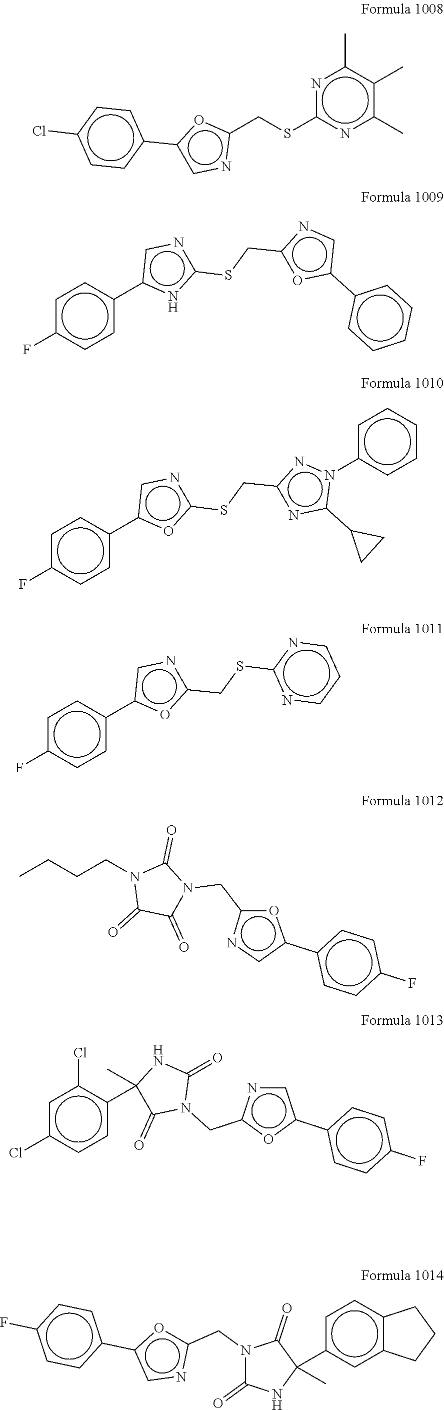

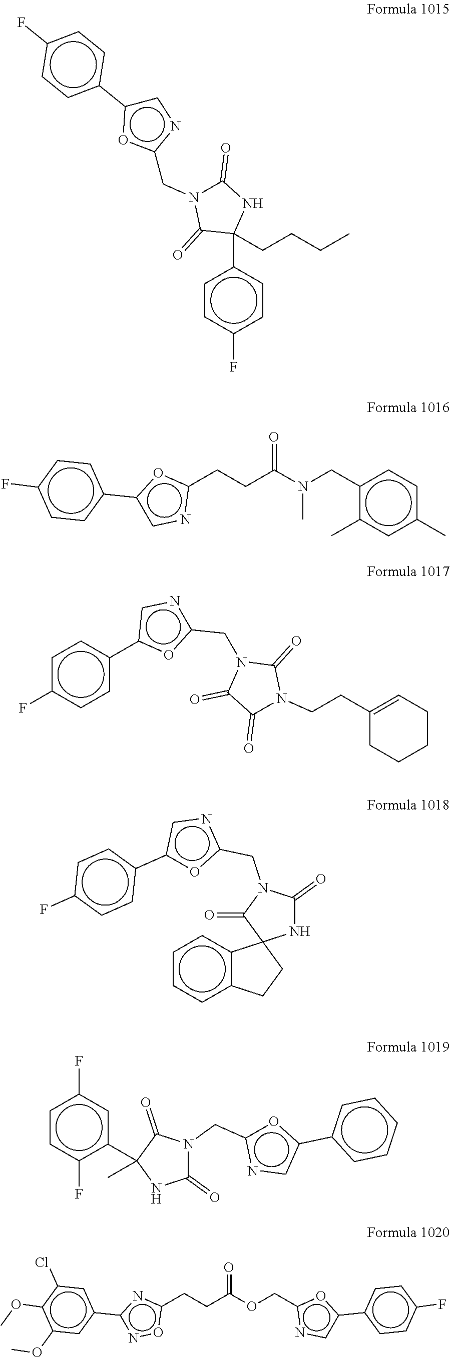

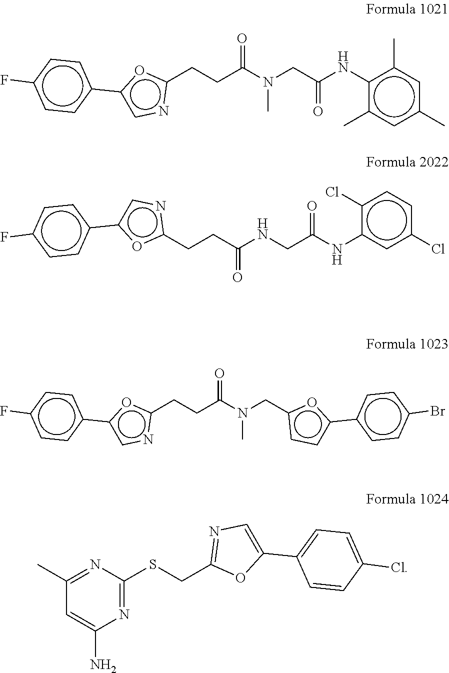

The disclosure also provides a compound of any one of Formulas 2001 to 2025, or a pharmaceutically acceptable analog, derivative, salt, solvate, hydrate, cocrystal, or prodrug thereof, wherein in some embodiments, the compound is an ANAT inhibitor:

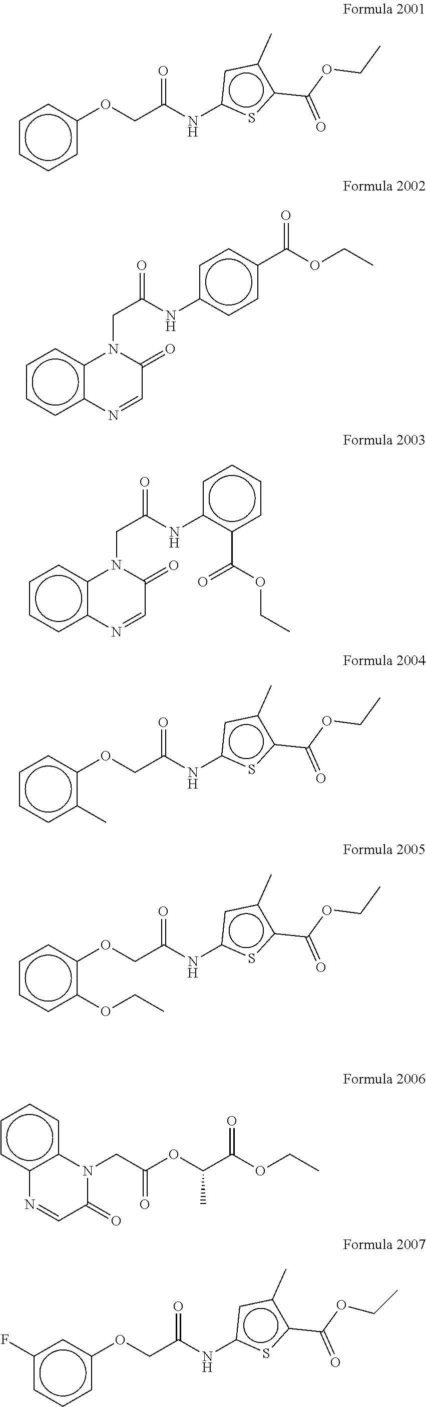

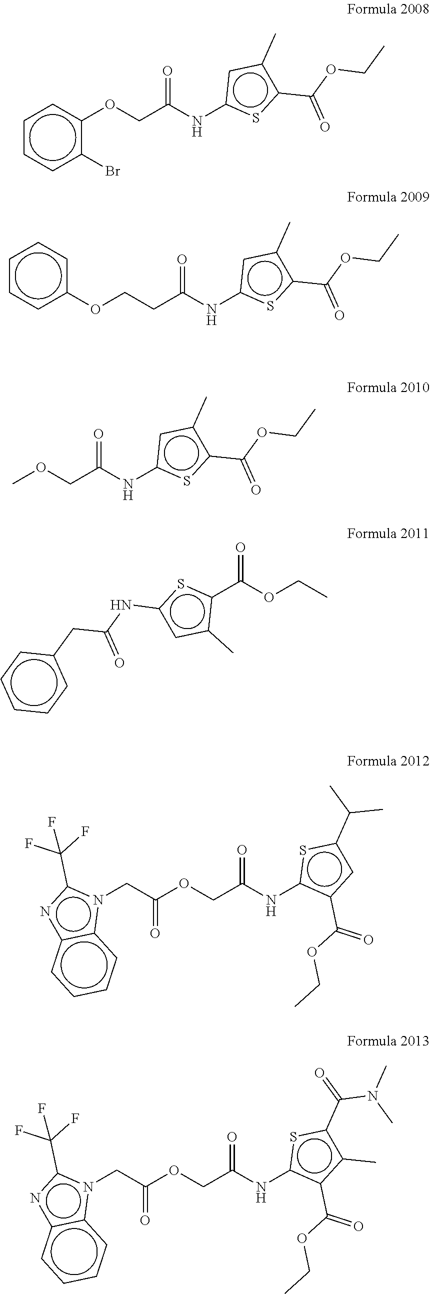

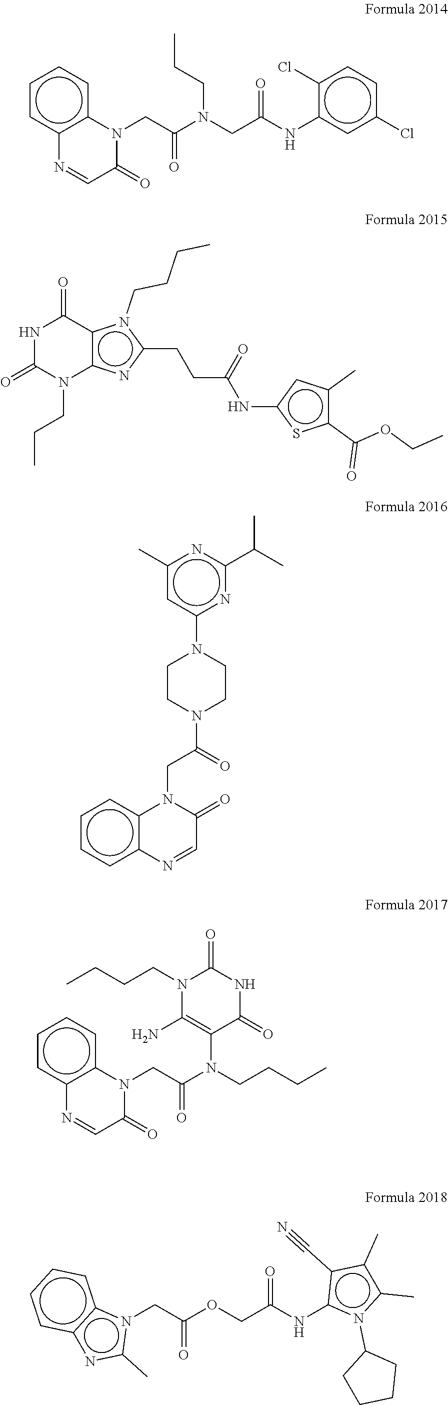

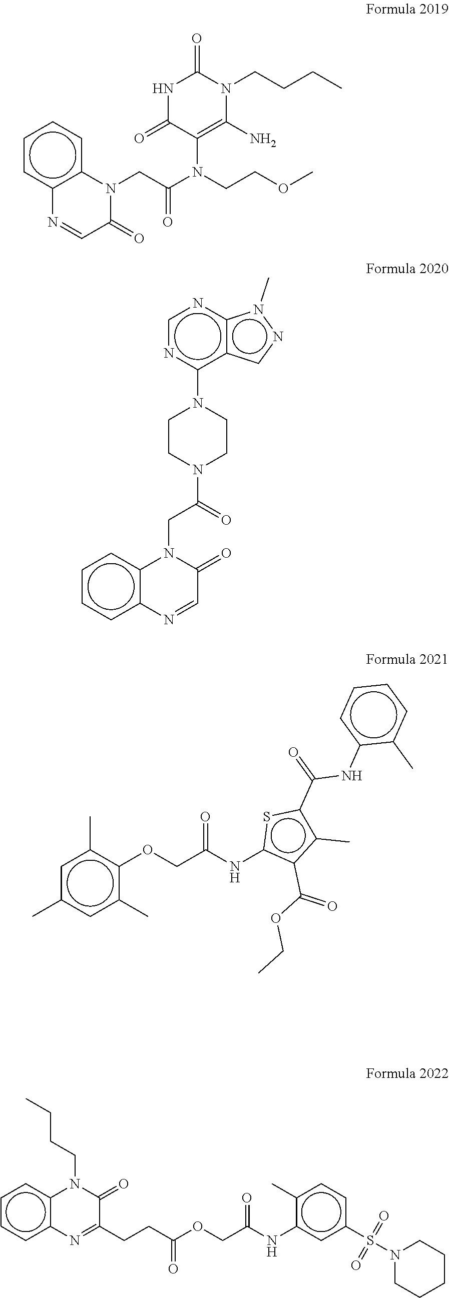

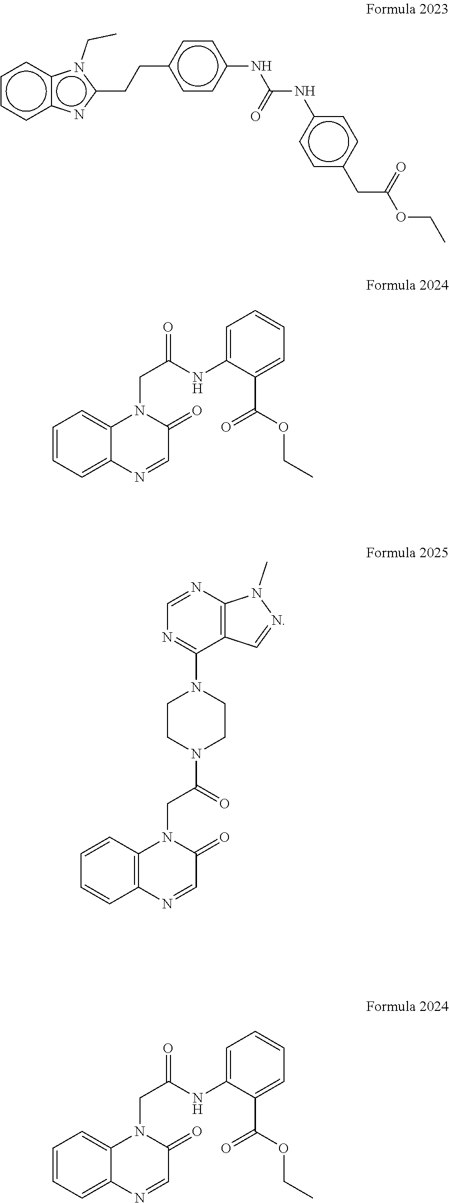

The disclosure also provides a compound of any one of Formulas 3001 to 3023, or a pharmaceutically acceptable analog, derivative, salt, solvate, hydrate, cocrystal, or prodrug thereof, wherein in some embodiments, the compound is an ANAT inhibitor.

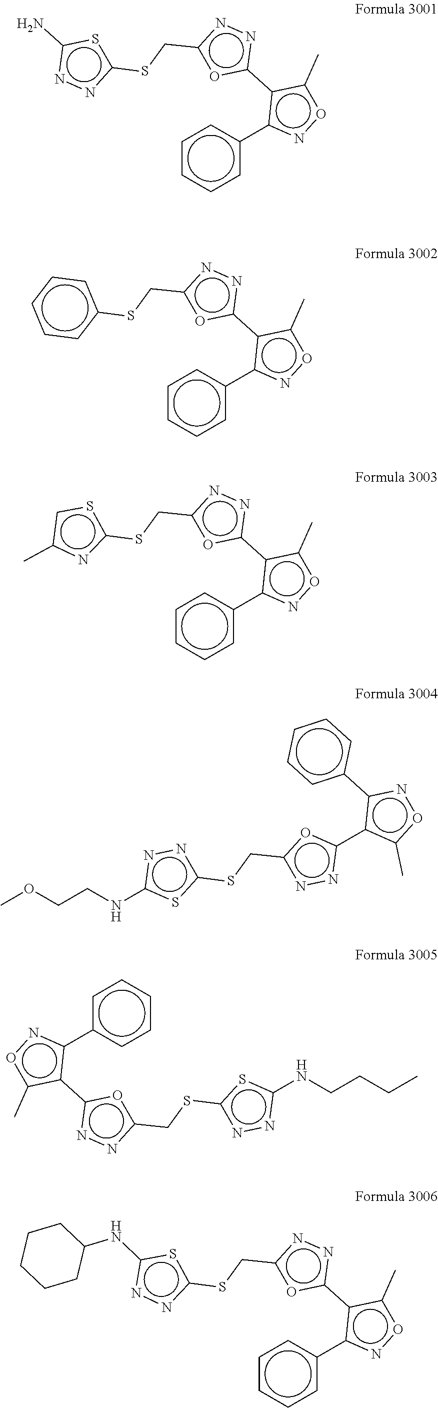

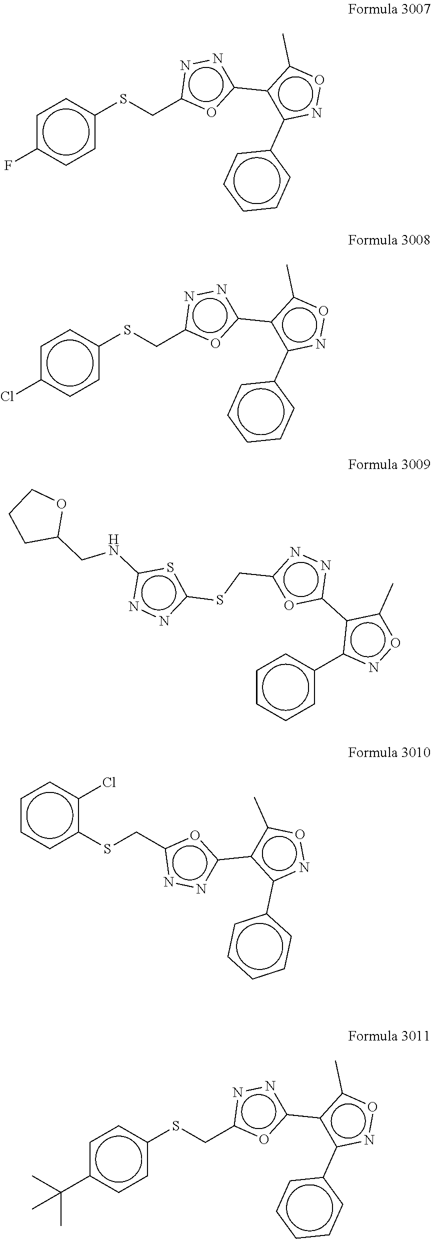

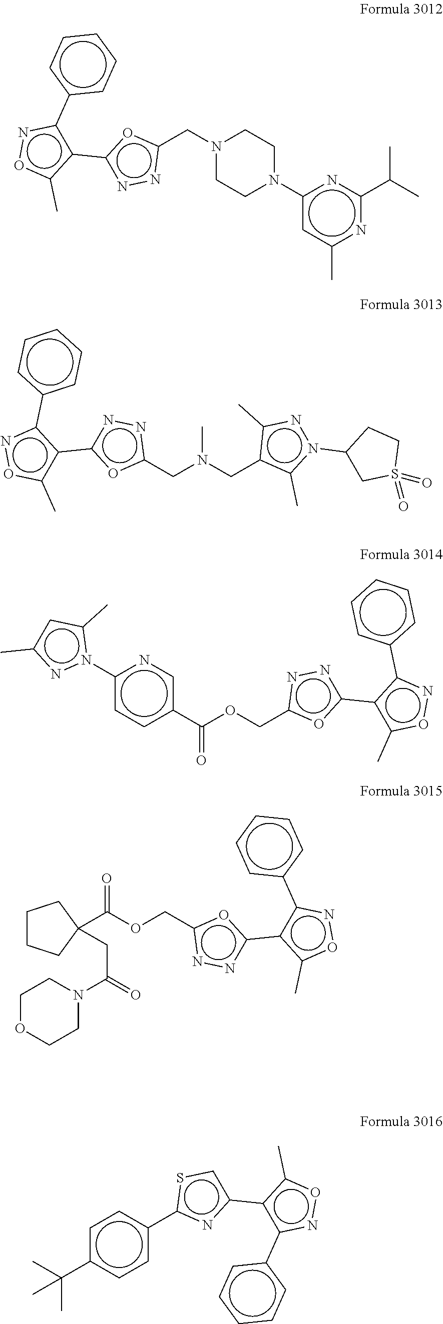

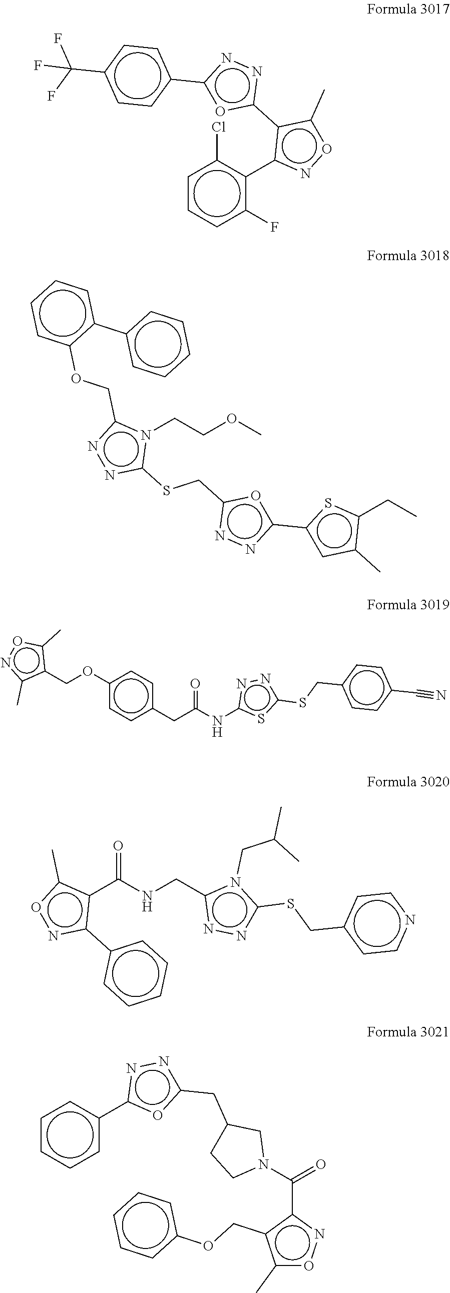

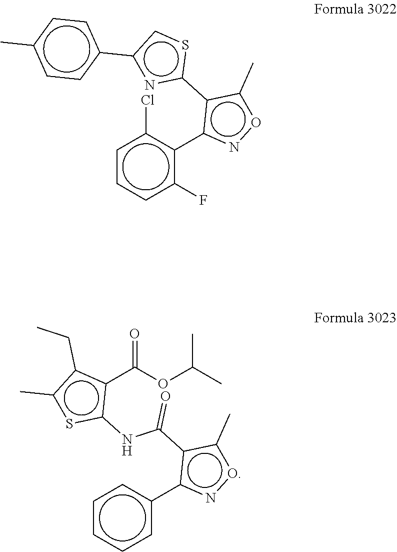

The disclosure also provides a compound of any one of Formulas 4001 to 4030, or a pharmaceutically acceptable analog, derivative, salt, solvate, hydrate, cocrystal, or prodrug thereof, wherein in some embodiments, the compound is an ANAT inhibitor:

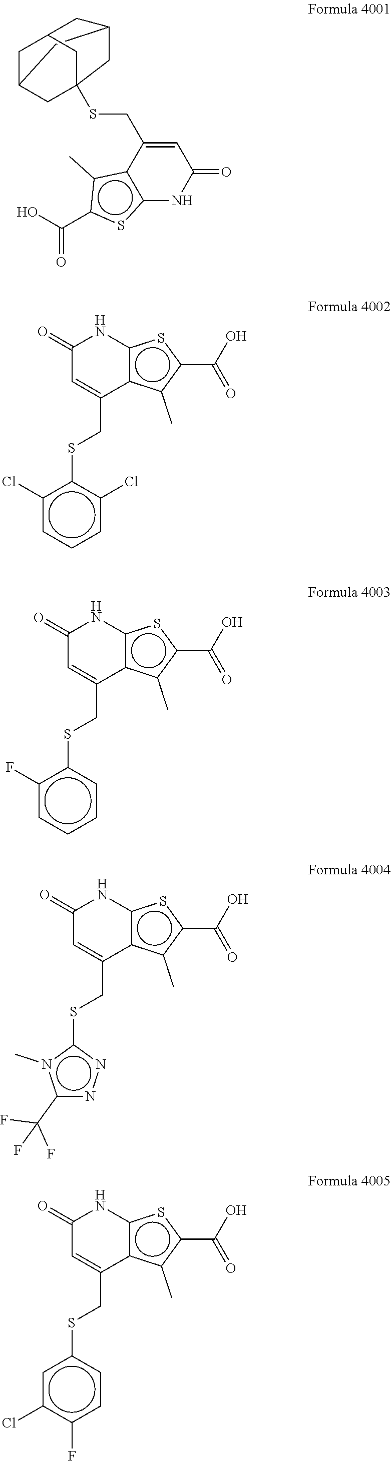

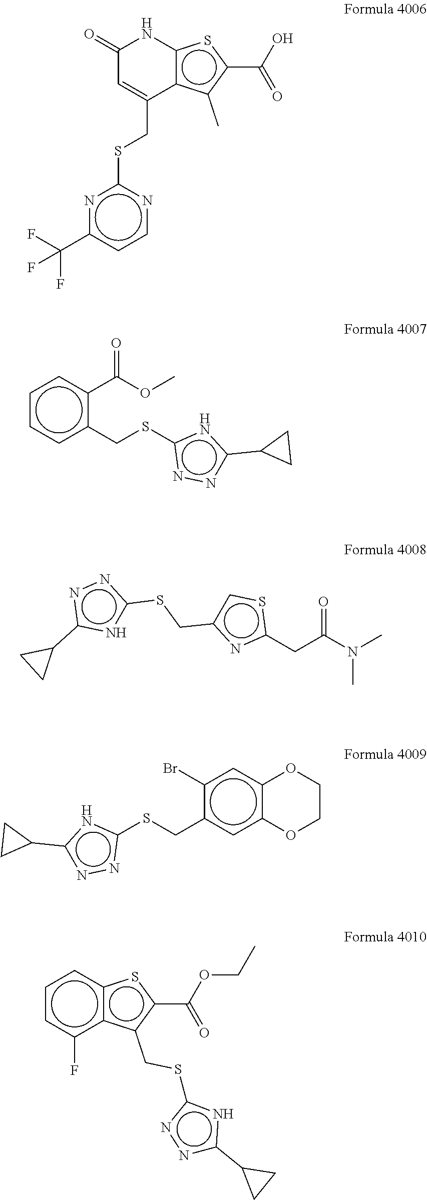

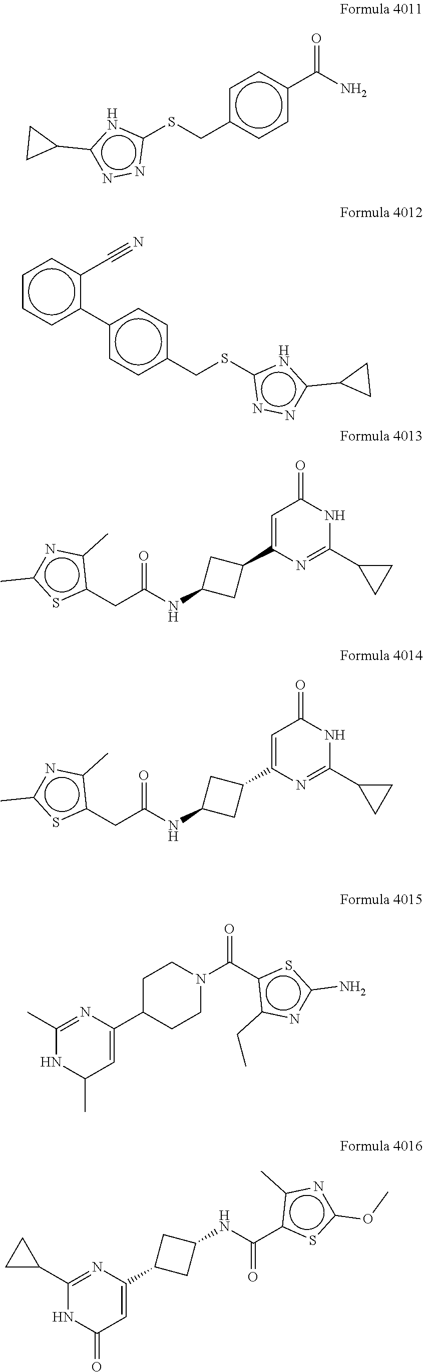

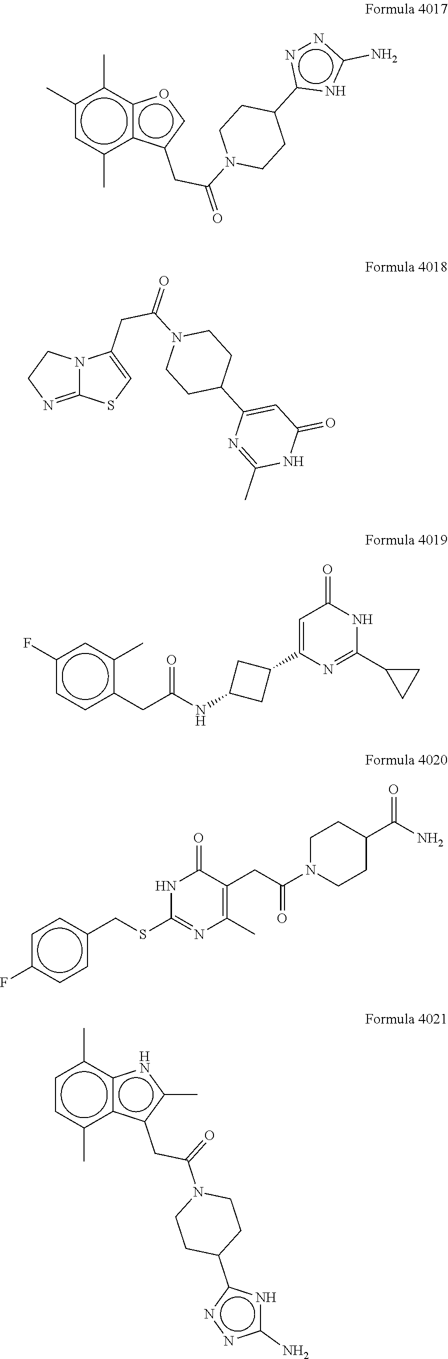

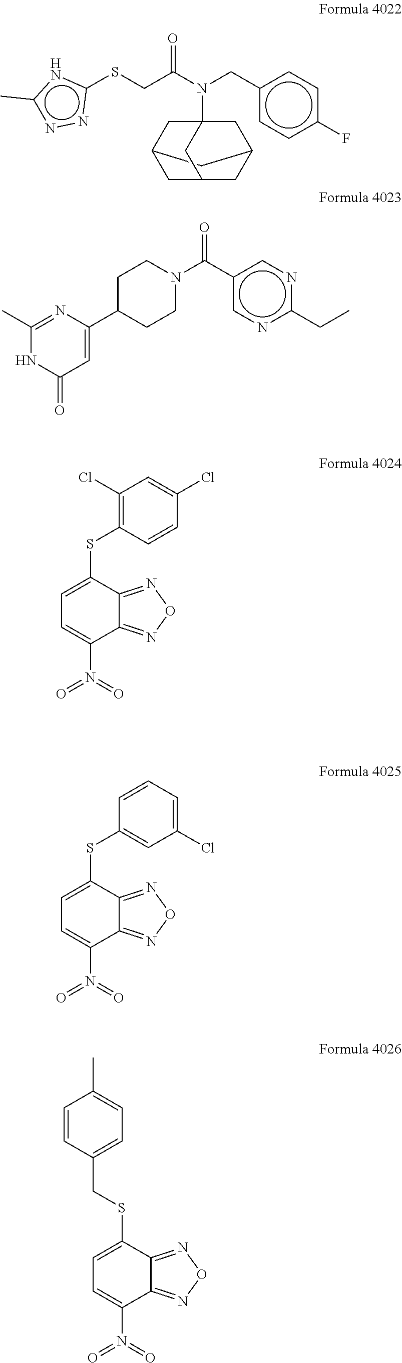

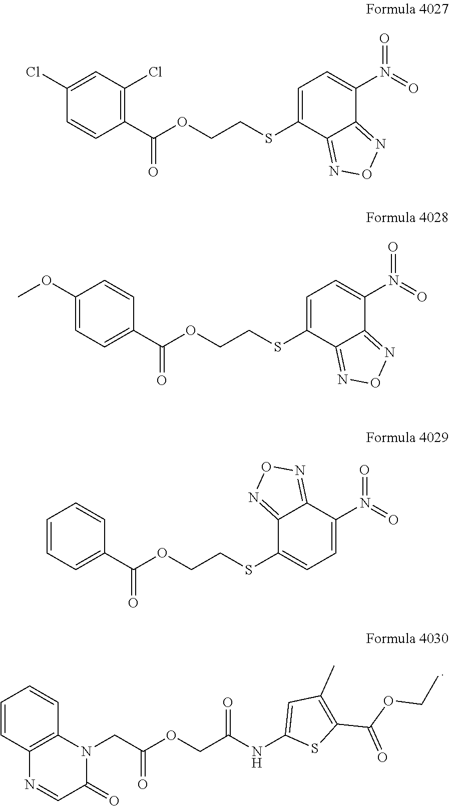 [SMILES: O=C(OCCSc1ccc([N+](=O)[O-])c2nonc12)c1ccc(Cl)cc1Cl ; COc1ccc(C(=O)OCCSc2ccc([N+](=O)[O-])c3nonc23)cc1 ; O=C(OCCSc1ccc([N+](=O)[O-])c2nonc12)c1ccccc1 ; CCOC(=O)c1sc(NC(=O)COC(=O)Cn2c(=O)cnc3ccccc32)cc1C]

The disclosure also provides a compound of any one of Formulas 5001 to 5023, or a pharmaceutically acceptable analog, derivative, salt, solvate, hydrate, cocrystal, or prodrug thereof, wherein in some embodiments, the compound is an ANAT inhibitor:

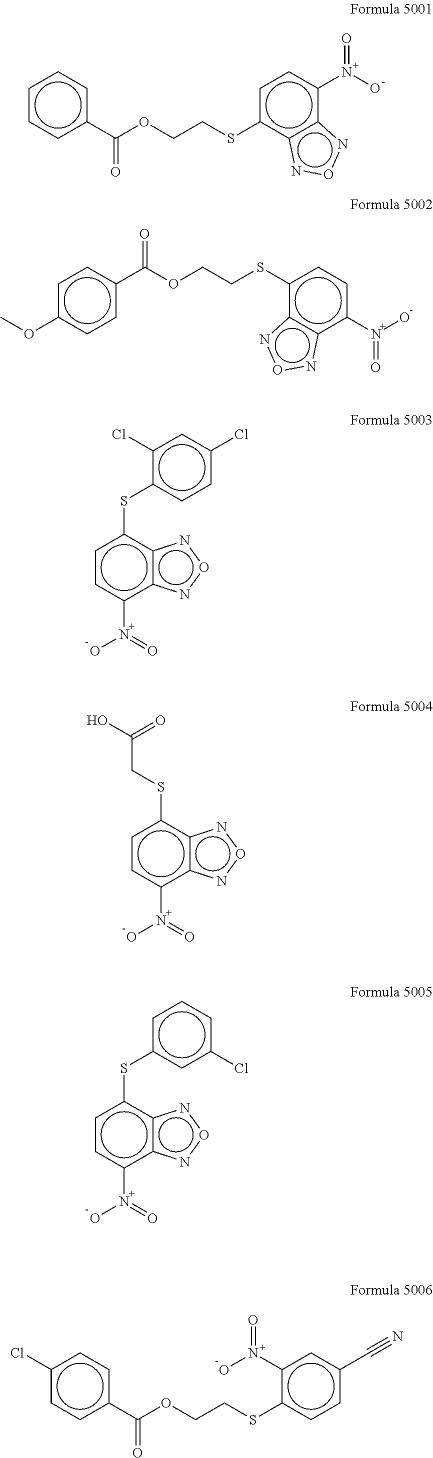

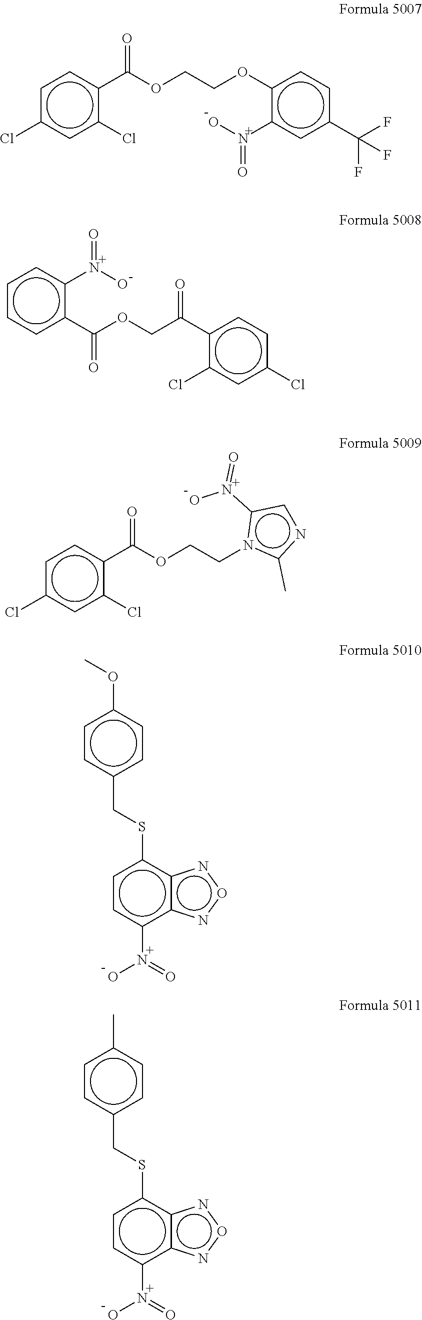

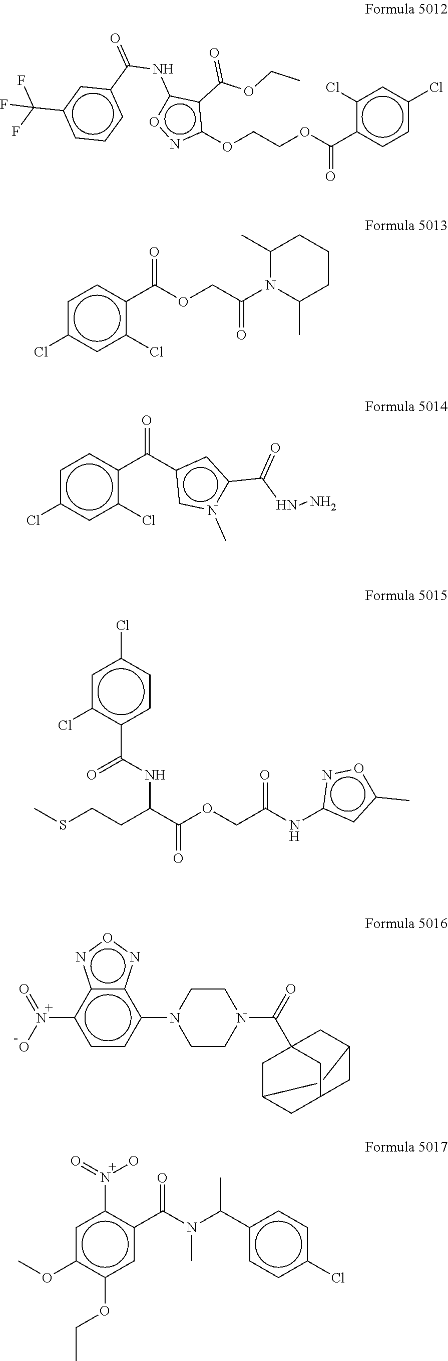

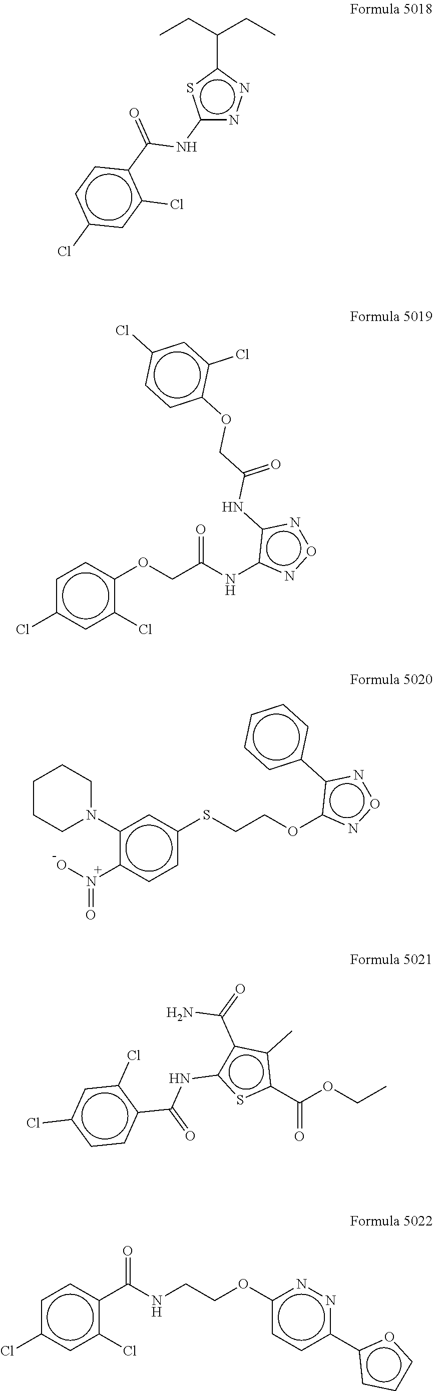

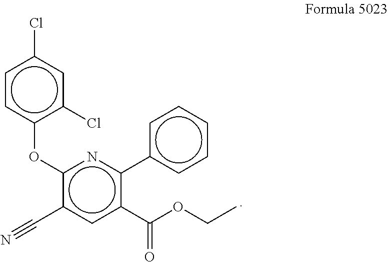

The disclosure also provides a compound of any one of Formulas 6001 to 6005, or a pharmaceutically acceptable analog, derivative, salt, solvate, hydrate, cocrystal, or prodrug thereof, wherein in some embodiments, the compound is an ANAT inhibitor:

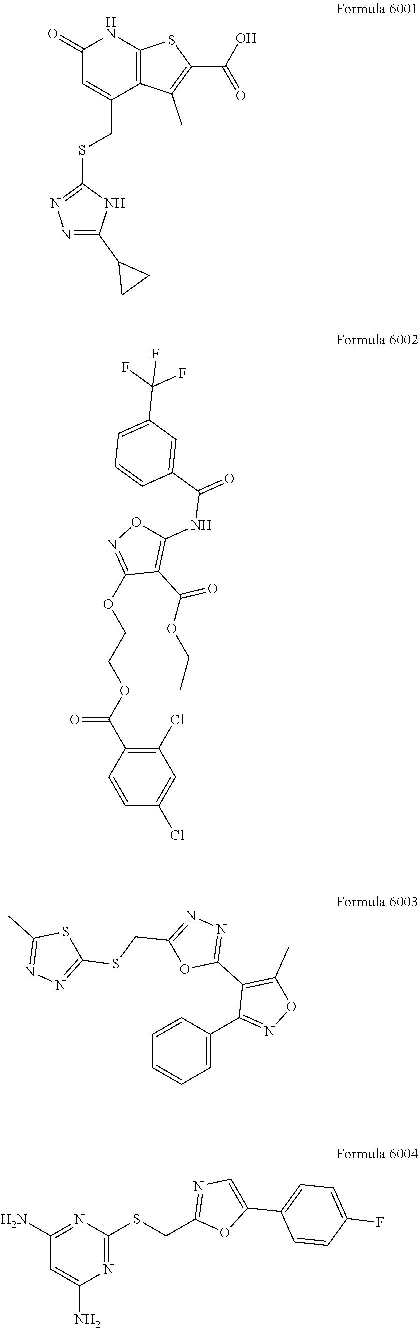 [SMILES: Cc1c(C(=O)O)sc2[nH]c(=O)cc(CSc3nnc(C4CC4)[nH]3)c12 ; CCOC(=O)c1c(OCCOC(=O)c2ccc(Cl)cc2Cl)noc1NC(=O)c1cccc(C(F)(F)F)c1 ; Cc1nnc(SCc2nnc(-c3c(-c4ccccc4)noc3C)o2)s1 ; Nc1cc(N)nc(SCc2ncc(-c3ccc(F)cc3)o2)n1]

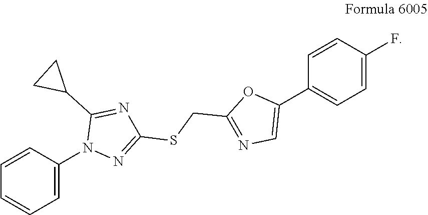 [SMILES: Fc1ccc(-c2cnc(CSc3nc(C4CC4)n(-c4ccccc4)n3)o2)cc1]

### Methods of Treatment

The compounds and compositions described herein can be used in methods for treating diseases. In some embodiments, the compounds and compositions described herein can be used in methods for treating diseases associated with the upregulation of ANAT. The compounds and compositions described herein may also be used in treating other disorders as described herein and in the following paragraphs.

In some embodiments, the hyperproliferative disorder described herein is cancer. In some embodiments, the cancer is lung cancer. In some embodiments, the hyperproliferative disorder treated by the compounds and compositions described herein includes cells having ANAT expression.

Canavan disease is a rare autosomal recessive leukodystrophy caused by mutations in the ASPA gene coding for the aspartoacylase enzyme that deacetylates N-acetylaspartate. N-acetylaspartate is one of the most prevalent small molecules in the brain, estimated to make up more than 0.1% of the brain by weight, and has been shown to be critical for brain development and homeostasis. The enzyme deficiency leads to a linear increase in brain N-acetylaspartate concentrations over time, raising it up to 14 mM, compared to the normal range of 3-8 mM. In addition, in an original characterization study, the levels of N-acetylaspartate in patients' urine were up to 200 times higher than normal. Following the cloning of the gene, it was shown that single nucleotide substitutions at residue 285 of the aspartoacylase enzyme account for up to 85% of the cases, pointing to the catalytic significance of the residue. Common symptoms of the disease include mental retardation, muscle hypotonia, and blindness. The disease has been reported worldwide but has an increased prevalence in the Ashkenazi Jewish population, with an incidence as high as 1:6,400.

N-acetylaspartate synthetase is an ER-associated, membrane-bound enzyme encoded by the NAT8L gene, that synthesizes N-acetylaspartate. Recent work has established in murine models that constitutive genetic ablation of the N-acetylaspartate synthetase enzyme leads to a decrease in N-acetylaspartate levels, fully reversing the pathology including spongy myelin and axonal degeneration. Further, heterozygous NAT8L^{-/-} animals accumulated less N-acetylaspartate but also showed almost normal survival times, indicating that partial pharmacological inhibition of the enzyme function could be an attractive therapeutic strategy. Similar results were achieved with intracerebroventricular administration of a viral vector carrying shRNA targeting Nat8l.

There are no small molecule treatments for Canavan disease. The only treatments currently available are gene therapies that are still in their infancy and provide variable results. Recent advances have made significant progress towards the study of the N-acetylaspartate synthetase function by purifying and kinetically characterizing the protein. Additionally, recent publications have reported the identification of two chemical scaffolds capable of nanomolar inhibition. The hypothesis of this proposal is that modulation of N-acetylaspartate synthetase function can alleviate the pathology and symptoms of the aspartoacylase deficiency. Any compounds with functional activity will serve as tools for further investigation of the N-acetylaspartate synthetase function or as starting points for therapeutic development.

Virtual Screen

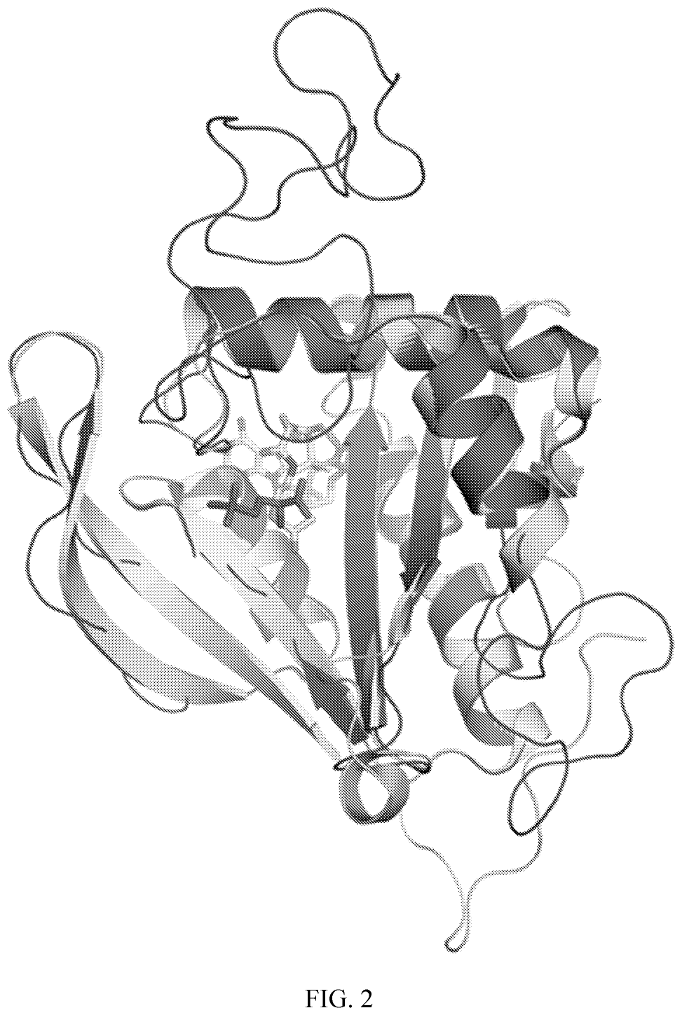

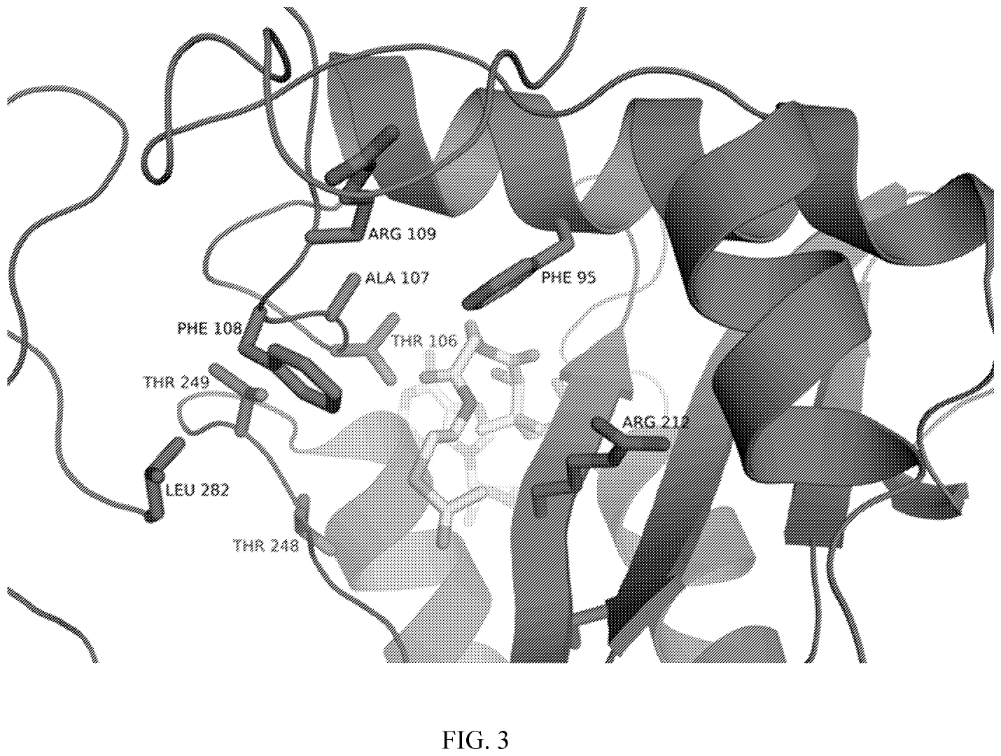

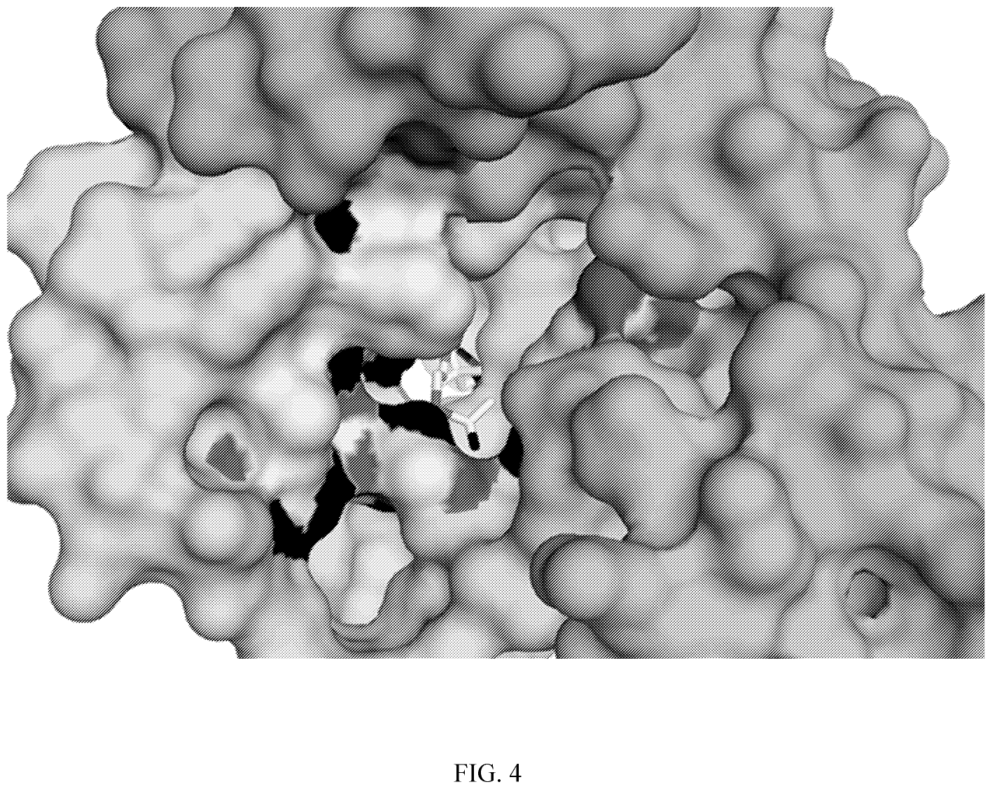

Given the challenges of studying membrane-associated enzymes, there are currently no publicly available structures of the human N-acetylaspartate synthetase. The closest homologues with determined structures include the putative Bacillus anthracis streptothricin acetyltransferase (PDB ID 3PP9, 25%), the putative Agrobacterium fabrum acetyltransferase (PDB ID 2GE3, 25%), and Streptomyces hygroscopicus phosphinothricin N-acetyltransferase (PDB ID 5T7E, 20%) (FIG. 1B). Like the reactions catalyzed by these distantly homologous proteins, human N-acetylaspartate synthetase relies on acetyl coenzyme A as the acetyl group donor to perform its catalysis function (FIGS. 2-4). Considering the resolution, the coverage, the cofactors, and the predicted secondary structure elements of the regions directly surrounding the aspartic acid binding site, PDB ID 5T7E was selected as the template for comparative structure modeling. The ligand binding site is defined by the following residues: F95, T106, A107, F108, R109, R212, T248, T249, L282.

Provided herein is a method of screening a molecular library of several million compounds using AtomNet, a proprietary technology (Atomwise). AtomNet is the first deep learning neural network for structure-based drug design and discovery. Its speed and accuracy make it the most advanced technology for small molecule binding affinity prediction. Top scoring compounds will be clustered and subsequently filtered for favorable properties to arrive at a final subset of deliverable compounds.

The disclosure provides methods of treating or preventing diseases alleviated by inhibiting ANAT in a subject in need thereof, comprising administering to the patient a therapeutically effective amount of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, or a pharmaceutically acceptable analog, derivative, salt, solvate, hydrate, cocrystal, or prodrug thereof. In some embodiments, the disease is Canavan disease. In some embodiments, the disease is cancer. In some embodiments, the disease is lung cancer. In some embodiments, the disease is an inborn error of metabolism. In some embodiments, the disease is epilepsy. In some embodiments, the disease is leukodystrophy. In some embodiments, the disease is N-acetyl aspartate deficiency. In some embodiments, the subject is human. In some embodiments, the ANAT inhibitor is administered in a dosage unit form. In some embodiments, the dosage unit comprises a physiologically compatible carrier medium.

The disclosure also provides method of treating or preventing disease alleviated by inhibiting ANAT in a subject in need thereof, comprising administering to the patient a therapeutically effective amount of a pharmaceutical composition comprising a therapeutically effective amount of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, or a pharmaceutically acceptable analog, derivative, salt, solvate, hydrate, cocrystal, or prodrug thereof. In some embodiments, the disease is Canavan disease. In some embodiments, the disease is cancer. In some embodiments, the disease is lung cancer. In some embodiments, the disease is an inborn error of metabolism. In some embodiments, the disease is epilepsy. In some embodiments, the disease is leukodystrophy. In some embodiments, the disease is N-acetyl aspartate deficiency. In some embodiments, the subject is human. In some embodiments, the ANAT inhibitor is administered in a dosage unit form. In some embodiments, the dosage unit comprises a physiologically compatible carrier medium.

Efficacy of the methods, compounds, and combinations of compounds described herein in treating, preventing and/or managing the indicated diseases or disorders can be tested using various animal models known in the art. For example, methods for determining efficacy of treatments for pancreatic cancer are described in Herreros-Villanueva, et al., World J. Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for ovarian cancer are described, e.g., in Mullany, et al., Endocrinology 2012, 153, 1585-92; and Fong, et al., J. Ovarian Res. 2009, 2, 12. Models for determining efficacy of treatments for melanoma are described, e.g., in Damsky, et al., Pigment Cell & Melanoma Res. 2010, 23, 853-859. Models for determining efficacy of treatments for lung cancer are described, e.g., in Meuwissen, et al., Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1, 32. Models for determining efficacy of treatments for colorectal cancer, including the CT26 model, are described in Castle, et al., BMC Genomics, 2013, 15, 190; Endo, et al., Cancer Gene Therapy, 2002, 9, 142-148; Roth et al., Adv. Immunol. 1994, 57, 281-351; Fearon, et al., Cancer Res. 1988, 48, 2975-2980.

### Pharmaceutical Compositions

The disclosure provides a pharmaceutical composition for use in the treatment of the diseases and conditions described herein. In some embodiments, the disclosure provides a pharmaceutical composition comprising a therapeutically effective amount of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, or a pharmaceutically acceptable analog, derivative, salt, solvate, hydrate, cocrystal, or prodrug thereof. In some embodiments, the disease is Canavan disease. In some embodiments, the disease is cancer. In some embodiments, the disease is lung cancer. In some embodiments, the disease is an inborn error of metabolism. In some embodiments, the disease is epilepsy. In some embodiments, the disease is leukodystrophy. In some embodiments, the disease is N-acetyl aspartate deficiency. In some embodiments, the subject is human. In some embodiments, the ANAT inhibitor is administered in a dosage unit form. In some embodiments, the dosage unit comprises a physiologically compatible carrier medium.

The pharmaceutical compositions are typically formulated to provide a therapeutically effective amount of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof as the active ingredient. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

The pharmaceutical compositions described above are preferably for use in the treatment of Canavan disease. In some embodiments, the pharmaceutical compositions described above are for use in the treatment of cancer, e.g., without limitation, lung cancer. In some embodiments, the pharmaceutical compositions described above are for use in the treatment of inborn errors of metabolism. In some embodiments, the pharmaceutical compositions described above are for use in the treatment of epilepsy. In some embodiments, the pharmaceutical compositions described above are for use in the treatment of leukodystrophy. In some embodiments, the pharmaceutical compositions described above are for use in the treatment of N-acetyl aspartate deficiency.

In some embodiments, the concentration of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, provided in the pharmaceutical compositions of the disclosure is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, provided in the pharmaceutical compositions of the disclosure is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, provided in the pharmaceutical compositions of the disclosure is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, provided in the pharmaceutical compositions of the disclosure is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, provided in the pharmaceutical compositions of the disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, provided in the pharmaceutical compositions of the disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the compounds provided according to the disclosure is effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting pharmaceutical compositions and methods for preparing the same.

### Pharmaceutical Compositions for Oral Administration

In preferred embodiments, the disclosure provides a pharmaceutical composition for oral administration containing an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, and a pharmaceutical excipient suitable for administration.

In preferred embodiments, the disclosure provides a solid pharmaceutical composition for oral administration containing an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, and a pharmaceutical excipient suitable for administration. In some embodiments, the composition further contains an effective amount of an additional active pharmaceutical ingredient. Such additional active pharmaceutical ingredients may also include those compounds used for sensitizing cells to additional agent(s).

In some embodiments, the pharmaceutical composition may be a liquid pharmaceutical composition suitable for oral consumption.

Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof, carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, 6-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In preferred embodiments, the disclosure provides a pharmaceutical composition for injection containing an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, and a pharmaceutical excipient suitable for injection. Components and amounts of compounds in the compositions are as described herein.

The forms in which the compositions of the disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In preferred embodiments, the disclosure provides a pharmaceutical composition for transdermal delivery containing an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, or a pharmaceutical composition of these compounds can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, can also be administered intraadiposally or intrathecally.

The compositions of the disclosure may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by any particular theory, compounds of the disclosure may slow or inhibit the migration and proliferation of smooth muscle cells in the arterial wall which contribute to restenosis. A compound of the disclosure may be administered, for example, by local delivery from the struts of a stent, from a stent graft, from grafts, or from the cover or sheath of a stent. In some embodiments, a compound of the disclosure is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. An ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable salts thereof, described herein, may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the disclosure in a suitable solvent, followed by evaporation of the solvent. Excess drug on the surface of the stent may be removed via an additional brief solvent wash. In yet other embodiments, compounds of the disclosure may be covalently linked to a stent or graft. A covalent linker may be used which degrades in vivo, leading to the release of the compound of the disclosure. Any bio-labile linkage may be used for such a purpose, such as ester, amide or anhydride linkages. An ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, may additionally be administered intravascularly from a balloon used during angioplasty. Extravascular administration of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, via the pericard or via advential application of formulations of the disclosure may also be performed to decrease restenosis.

Exemplary parenteral administration forms include solutions or suspensions of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, in sterile aqueous solutions, for example, aqueous propylene glycol or dextrose solutions. Such dosage forms can be suitably buffered, if desired.

The disclosure also provides kits. The kits include an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In some embodiments, an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, and another active pharmaceutical ingredient are provided as separate compositions in separate containers within the kit. In some embodiments, an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

The kits described above are preferably for use in the treatment of the diseases and conditions described herein. In a preferred embodiment, the kits are for use in the treatment of cancer or hyperproliferative disorders.

In an embodiment, the kits described herein are for use in the treatment of Canavan disease. In an embodiment, the kits described herein are for use in the treatment of cancer. In some embodiments, the kits described herein are for use in the treatment of lung cancer. In some embodiments, the kits described herein are for use in the treatment of inborn errors of metabolism. In some embodiments, the kits described herein are for use in the treatment of epilepsy. In some embodiments, the kits described herein are for use in the treatment of leukodystrophy. In some embodiments, the kits described herein are for use in the treatment of N-acetyl aspartate deficiency.

### Dosages and Dosing Regimens

The amounts of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, administered, will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, may be provided in units of mg/kg of body mass or in mg/m² of body surface area.

In some embodiments, an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein is administered in multiple doses. In a preferred embodiment, an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein is administered in multiple doses. Dosing may be once, twice, three times, four times, five times, six times, or more than six times per day. Dosing may be once a month, once every two weeks, once a week, or once every other day. In other embodiments, an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, is administered about once per day to about 6 times per day. In some embodiments, an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, is administered once daily, while in other embodiments, an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein is administered twice daily, and in other embodiments an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, is administered three times daily.

Administration a compound of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, may continue as long as necessary. In some embodiments, an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, is administered for more than 1, 2, 3, 4, 5, 6, 7, 14, or 28 days. In some embodiments, an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein is administered for less than 28, 14, 7, 6, 5, 4, 3, 2, or 1 day. In some embodiments, an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein is administered chronically on an ongoing basis—e.g., for the treatment of chronic effects. In another embodiment, an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, continues for less than about 7 days. In yet another embodiment, the administration continues for more than about 6, 10, 14, 28 days, two months, six months, or one year. In some cases, continuous dosing is achieved and maintained as long as necessary.

In some embodiments, an effective dosage of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg.

In some embodiments, an effective dosage of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of an ANAT inhibitor of any one of Formulas I to VII, any one of Formulas VIII-a to VIII-e, any one of Formulas 1 to 5, any one of Formulas 1001 to 1024, any one of Formulas 2001 to 2025, any one of Formulas 3001 to 3023, any one of Formulas 4001 to 4030, any one of Formulas 5001 to 5023, or any one of Formulas 6001 to 6005, and their features and limitations as described herein, or pharmaceutically acceptable analogs, derivatives, salts, solvates, hydrates, cocrystals, or prodrugs thereof, described herein, may be administered in either single or multiple doses by any of the accepted modes of administration of agents having similar utilities, including rectal, buccal, intranasal and transdermal routes, by intraarterial injection, intravenously, intraperitoneally, parenterally, intramuscularly, subcutaneously, orally, topically, or as an inhalant.

The following clauses describe certain embodiments.

Clause 1. A method of treating or preventing a disease alleviated by inhibiting ANAT in a subject in need thereof, comprising administering to the subject a therapeutically effective amount of a compound of Formula IV, Formula V, Formula I, Formula II, Formula III-a, Formula III-b, Formula VI, or Formula VII:

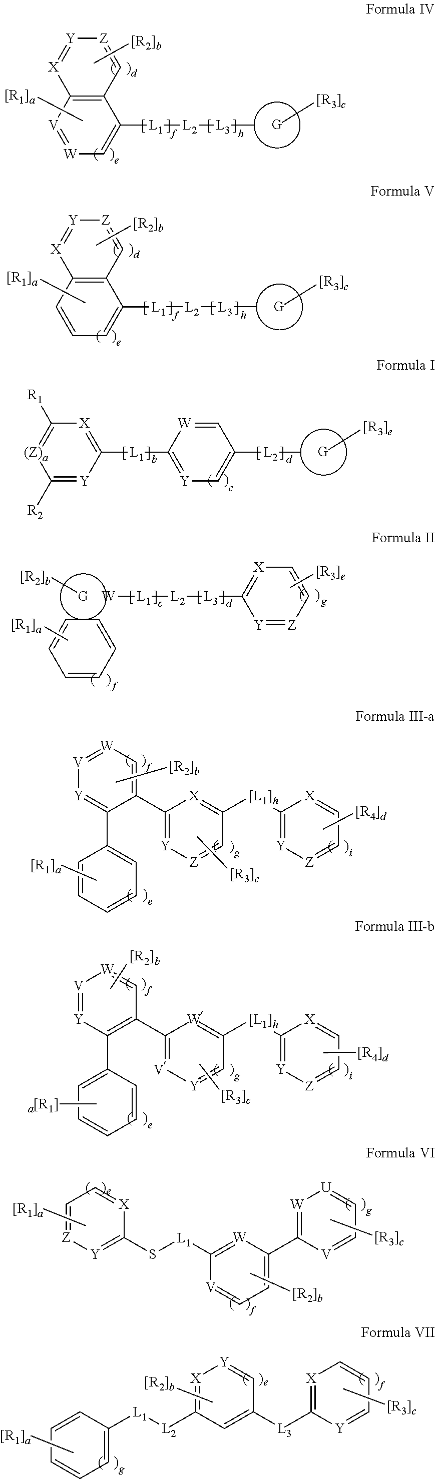

or a pharmaceutically acceptable analog, derivative, salt, solvate, hydrate, cocrystal, or prodrug thereof, wherein in Formulas I to VII:

U, V, W, V′, W′, Y′, X, Y, and Z are independently selected at each occurrence from C, CH, CH₂, N, NH, NR^{a}, O, S

G is independently at each occurrence a mono- or polycyclic optionally substituted cycloalkyl, mono- or polycyclic optionally substituted heterocycloalkyl, mono- or polycyclic optionally substituted aryl, mono- or polycyclic optionally substituted arylalkyl, mono- or polycyclic optionally substituted heteroaryl, and mono- or polycyclic optionally substituted heteroarylalkyl;

R₁, R₂, and R₃ are independently selected at each occurrence from hydrogen, optionally substituted alkylheteroaryl, optionally substituted alkyl, optionally substituted heteroalkyl, optionally substituted alkenyl, optionally substituted alkynyl, optionally substituted cycloalkyl, optionally substituted heterocycloalkyl, optionally substituted fused heterocycloalkyl, optionally substituted aryl, optionally substituted arylalkyl, optionally substituted heteroaryl, optionally substituted alkylheteroaryl, optionally substituted heteroarylalkyl, optionally substituted alkoxy, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂, wherein R₁, R₂, and R₃ can optionally be joined to form a carbo- or heterocycle; L₁, L₂, and L₃ are linkers comprising independently at each occurrence one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a} C(O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl;

wherein any optional substituent is independently selected at each occurrence from optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂;

R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and

a, b, c, d, e, f, g, h, and i are independently at each occurrence an integer selected from 0, 1, 2, 3, 4, 5, and 6.

Clause 2. The method of clause 1, wherein G is independently at each occurrence selected from benzene, pyridine, pyrimidine, pyrazine, pyridazine, and triazine.

Clause 3. The method of clause 1, wherein G is independently at each occurrence selected from furan, thiophene, pyrrole, thiazole, isothiazole, 1,2,3-thiadiazole, imidazole, oxazole, isoxazole, 1,2,3-oxadiazole, 1,3,4-oxadiazole, 1,2,4-oxadiazole, 1,2,3-triazole, 1,2,4-triazole, and pyrazole.

Clause 4. The method of clause 1, wherein G is independently at each occurrence selected from naphthalene, quinoline, isoquinoline, cinnoline, quinoxaline, phtalazine, pyridopyrazine, pteridine, pyridopyridazine, naphtyridine, carbazole, dibenzofuran, or quinazoline.

Clause 5. The method of clause 1, wherein G is independently at each occurrence selected from indole, benzoxazole, benzothiophene, benzimidazole, indazole, benzotriazole, pyrrolopyridine, triazolopyridine, purine, indolizine, pyrrolopyrimidine, pyrrolopyrazine, pyrrolopyrimine, pyrrolopyridazine, imidazopyridine, pyrazolopyridine, imidazopyridazine, or imidazopyrimidine.

Clause 6. The method of clause 1, wherein the compound has a formula selected from any one of Formulas 1 to 5:

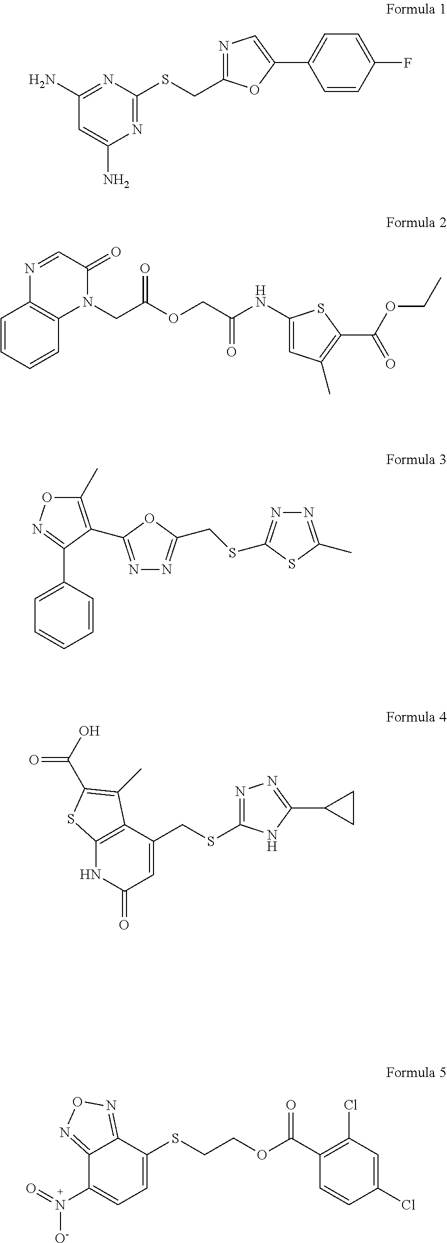

Clause 7. The method of clause 1, wherein the compound has a formula selected from any one of Formulas 1001 to 1024:

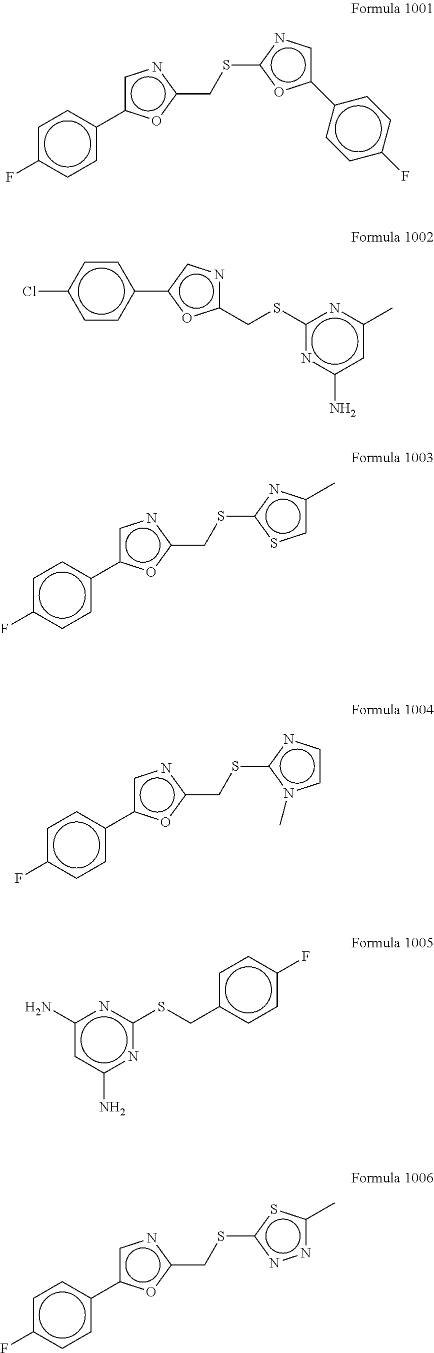

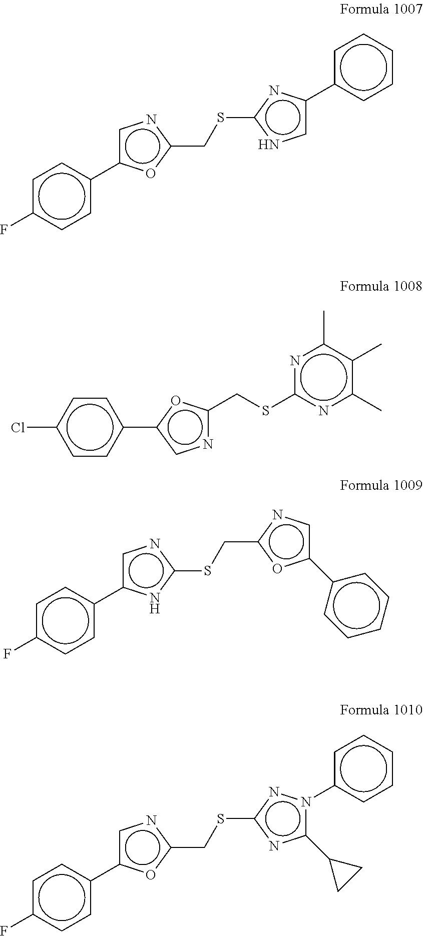

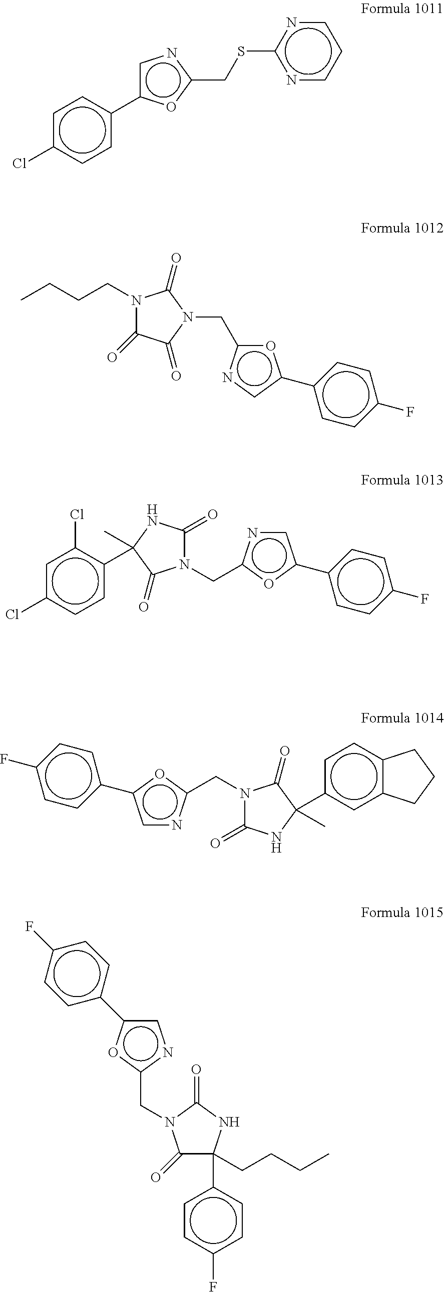

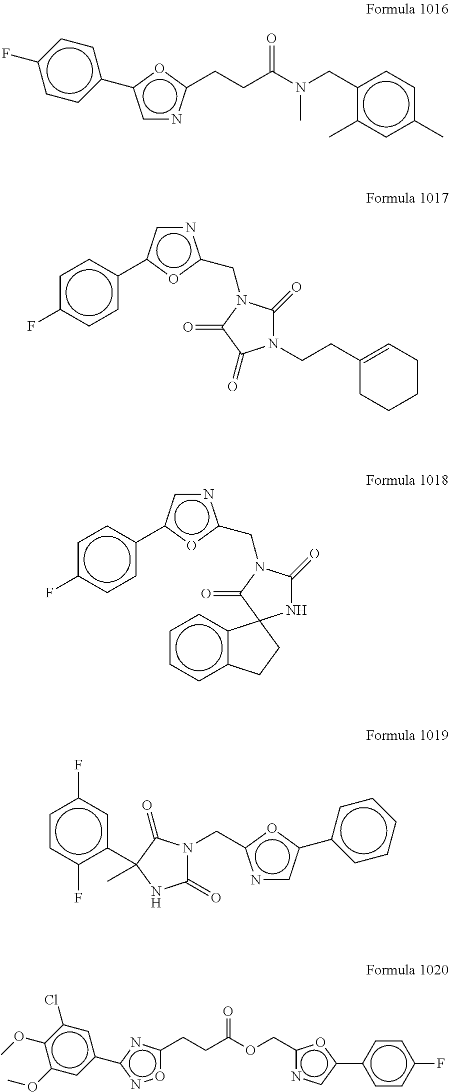

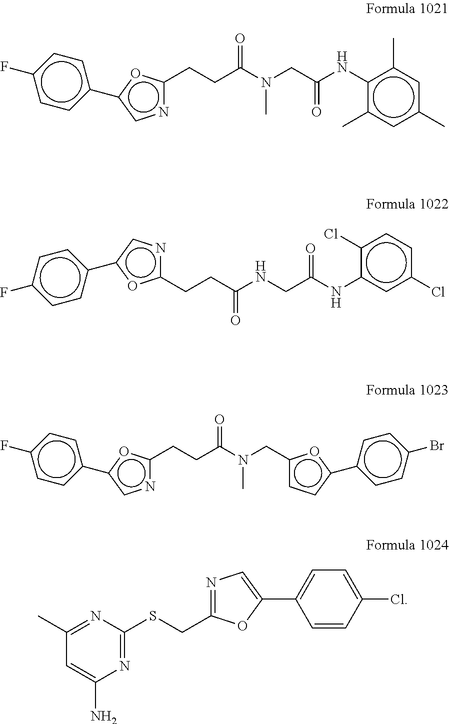

Clause 8. The method of clause 1, wherein the compound has a formula selected from any one of Formulas 2001 to 2025:

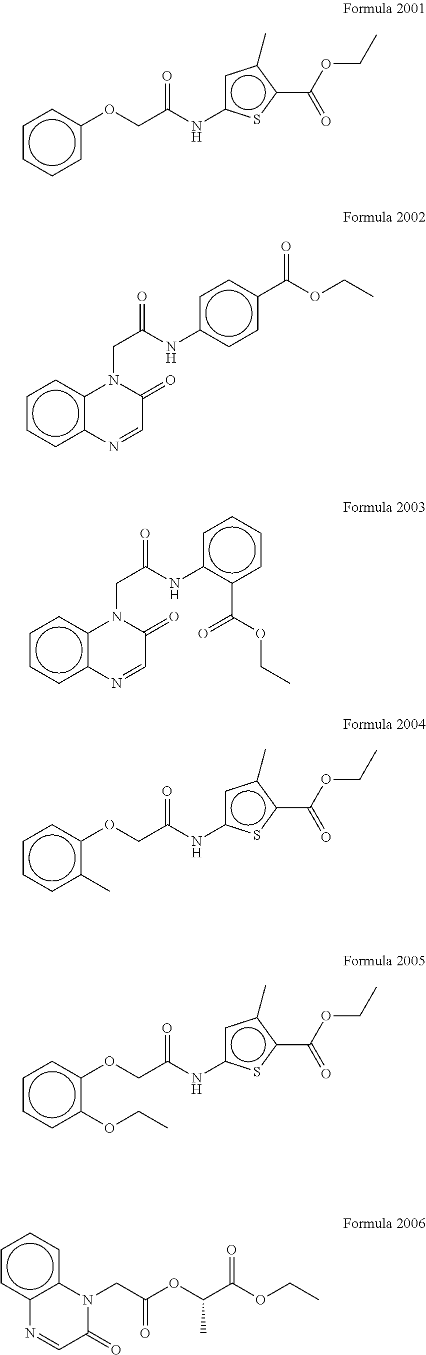

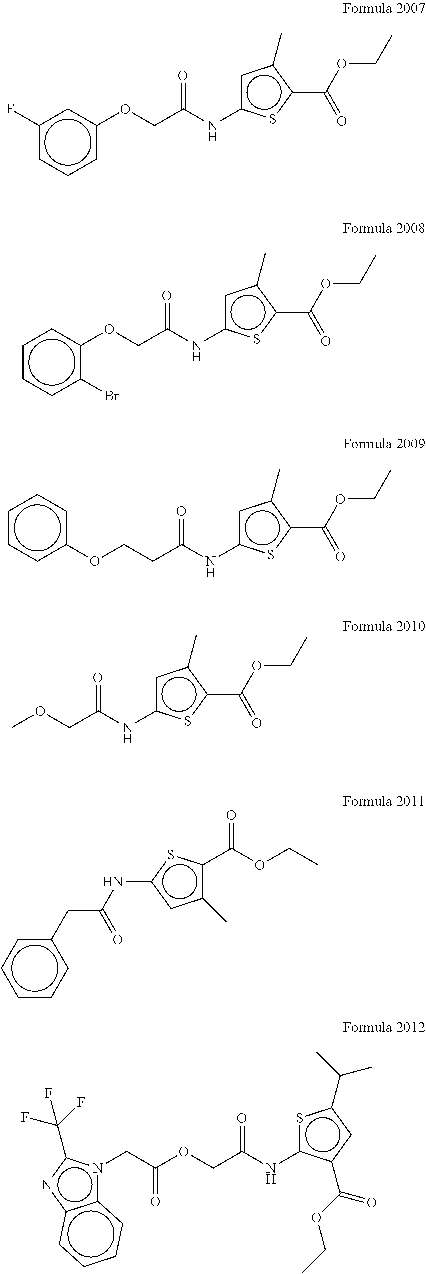

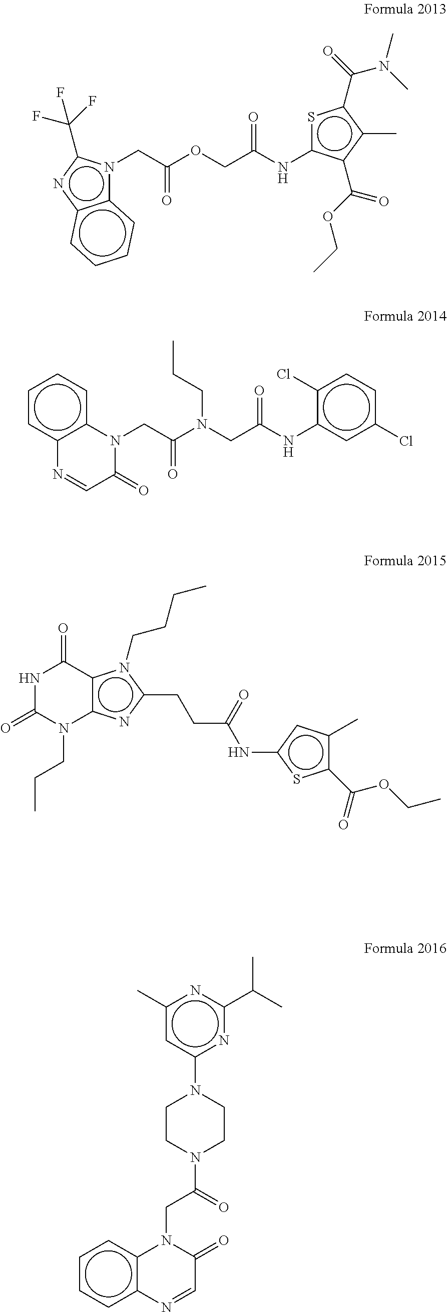

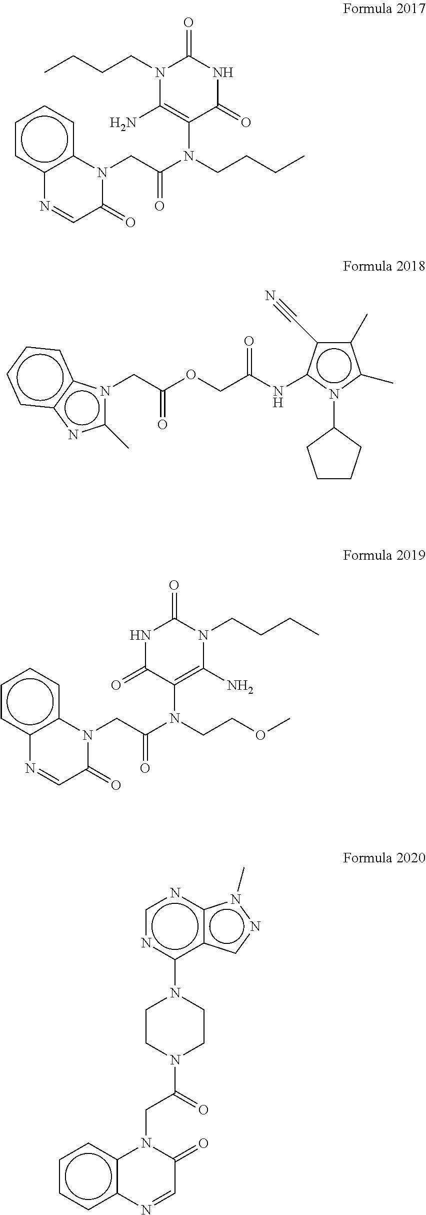

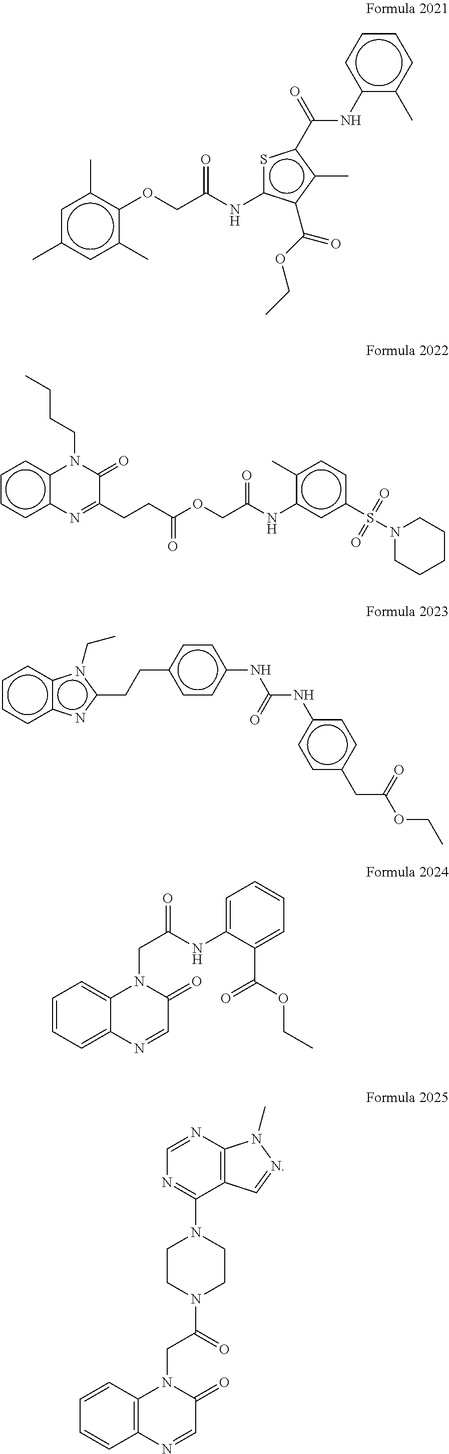

Clause 9. The method of clause 1, wherein the compound has a formula selected from any one of Formulas 3001 to 3023:

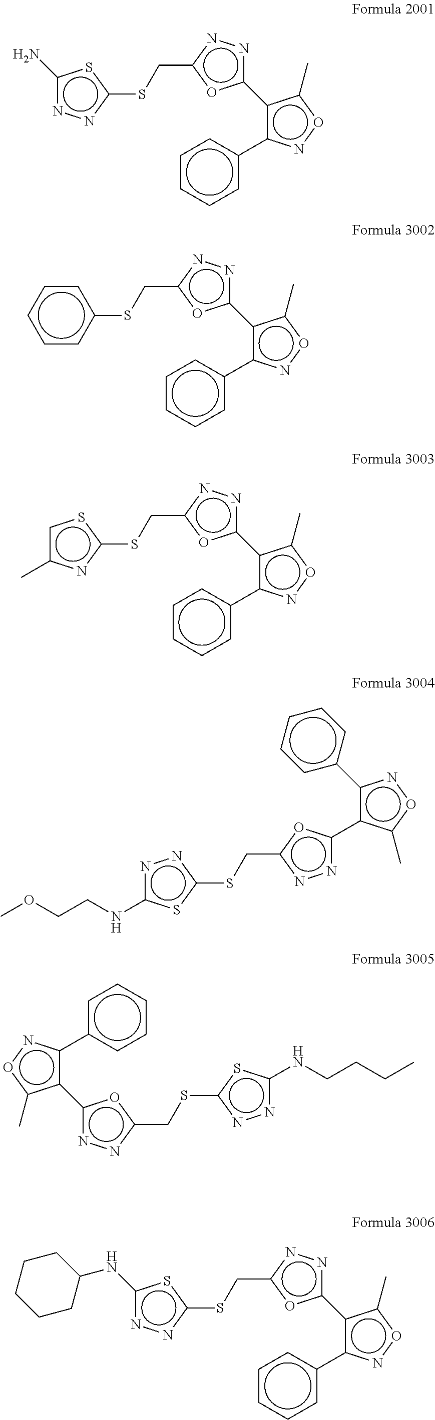

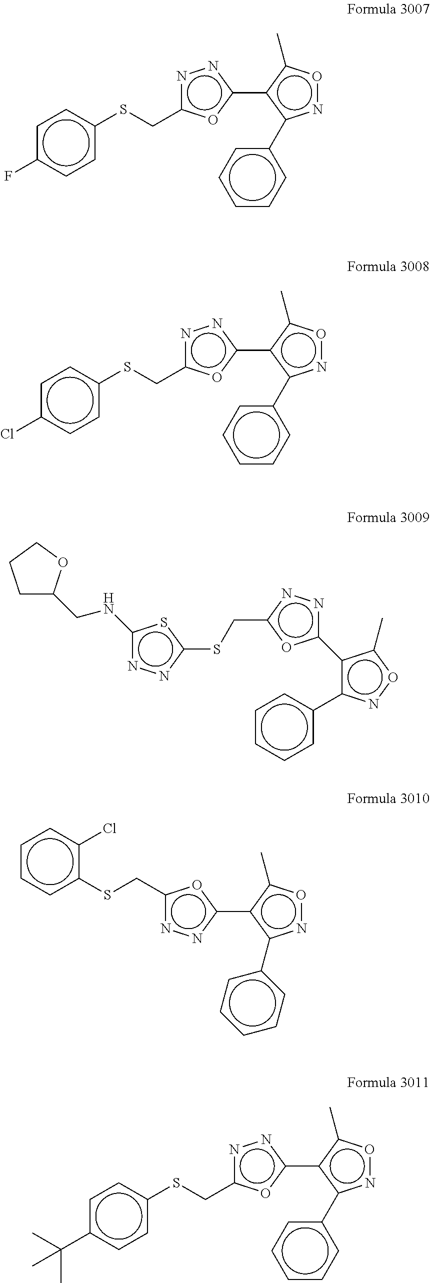

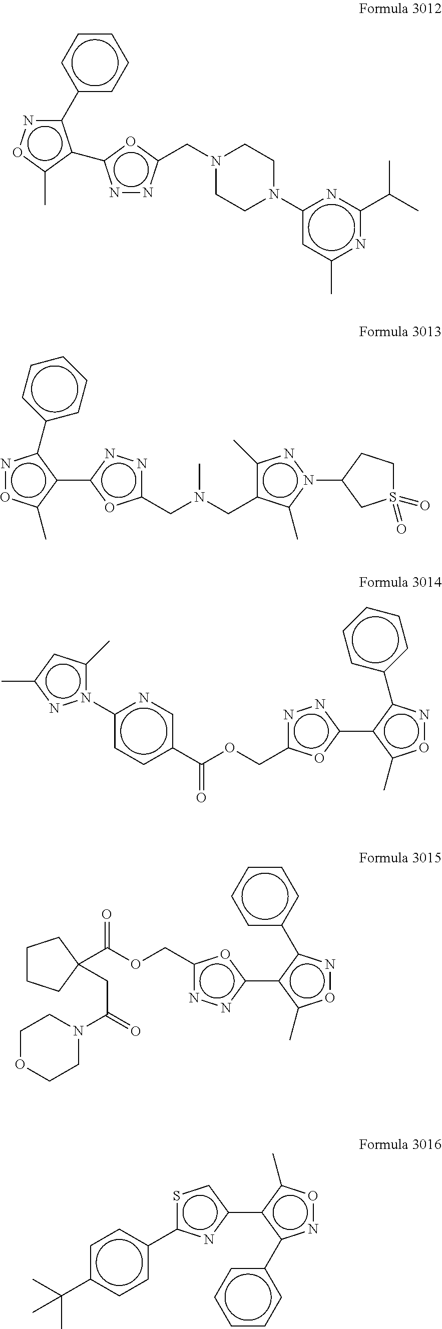

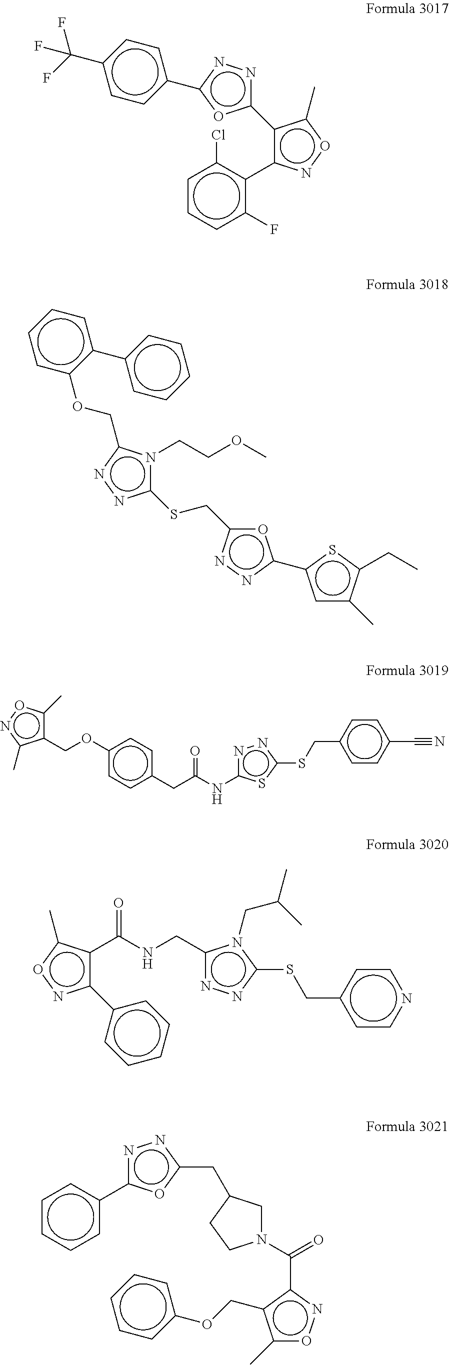

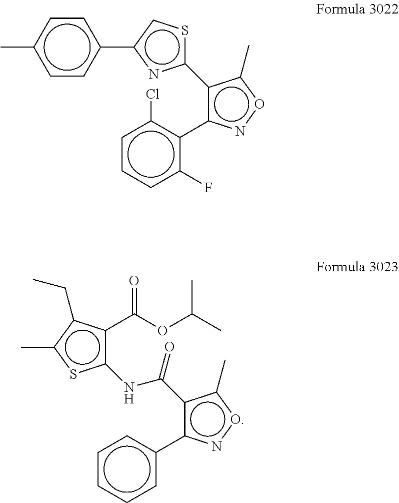

Clause 10. The method of clause 1, wherein the compound has a formula selected from any one of Formulas 4001 to 4030:

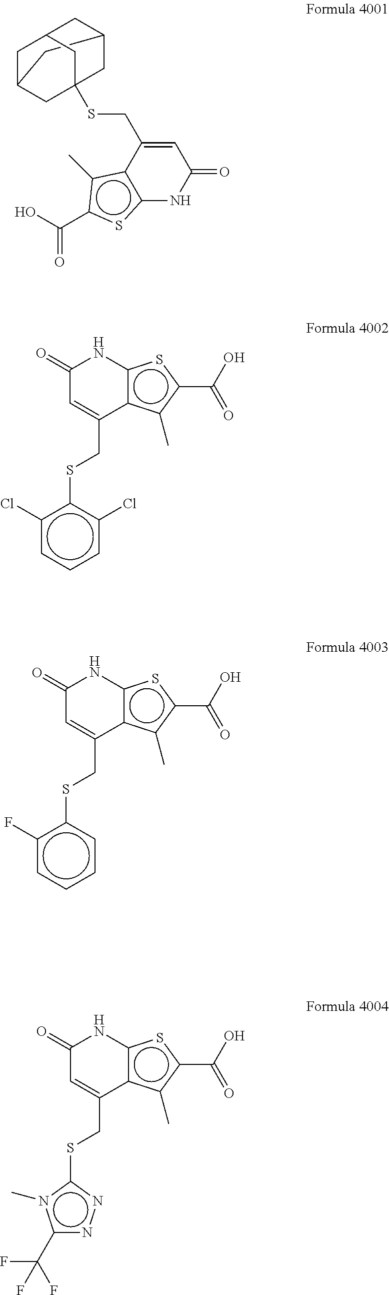

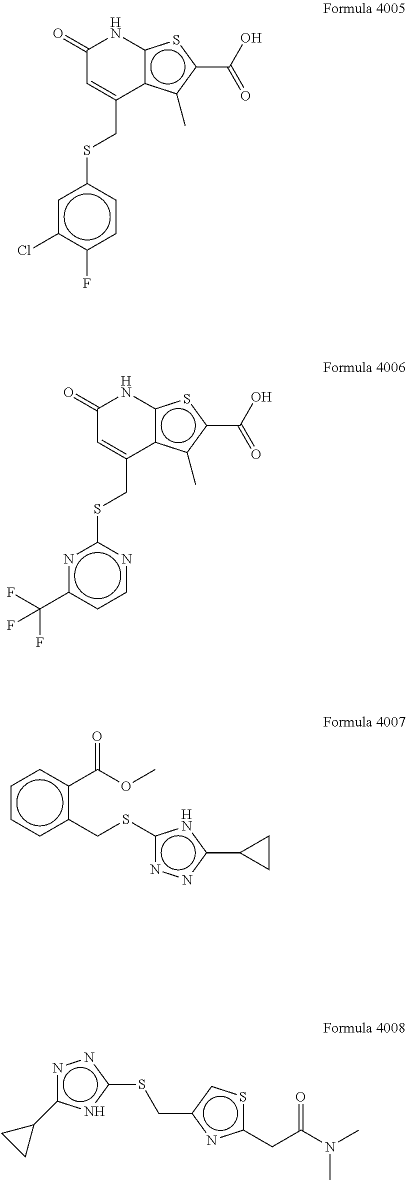

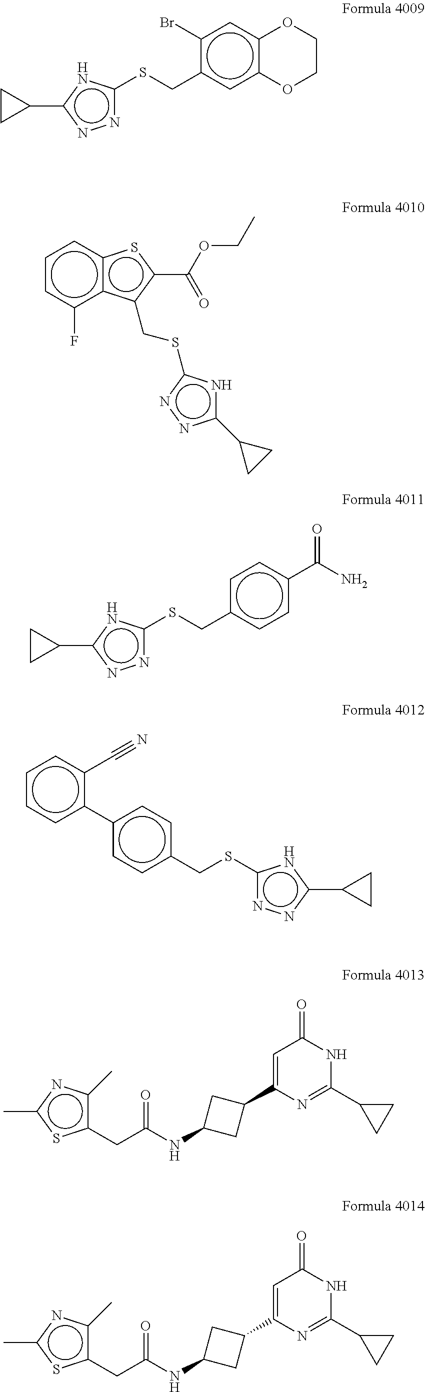

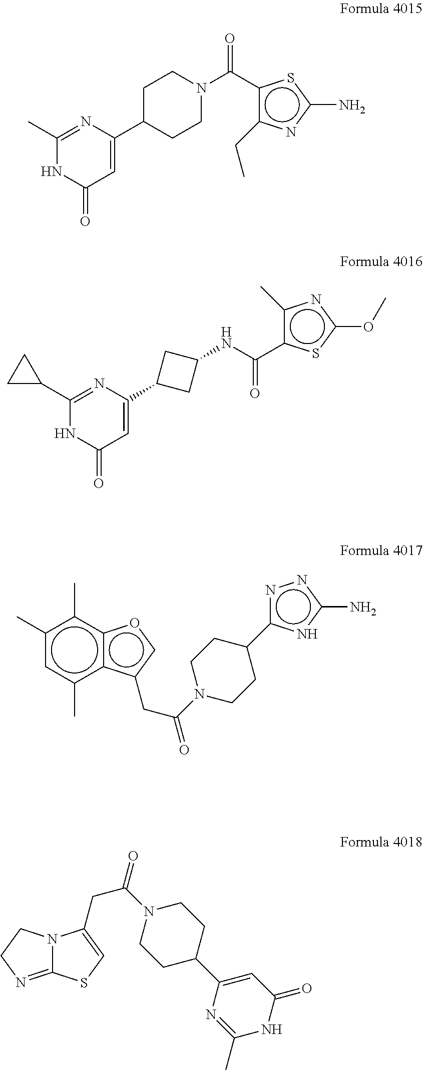

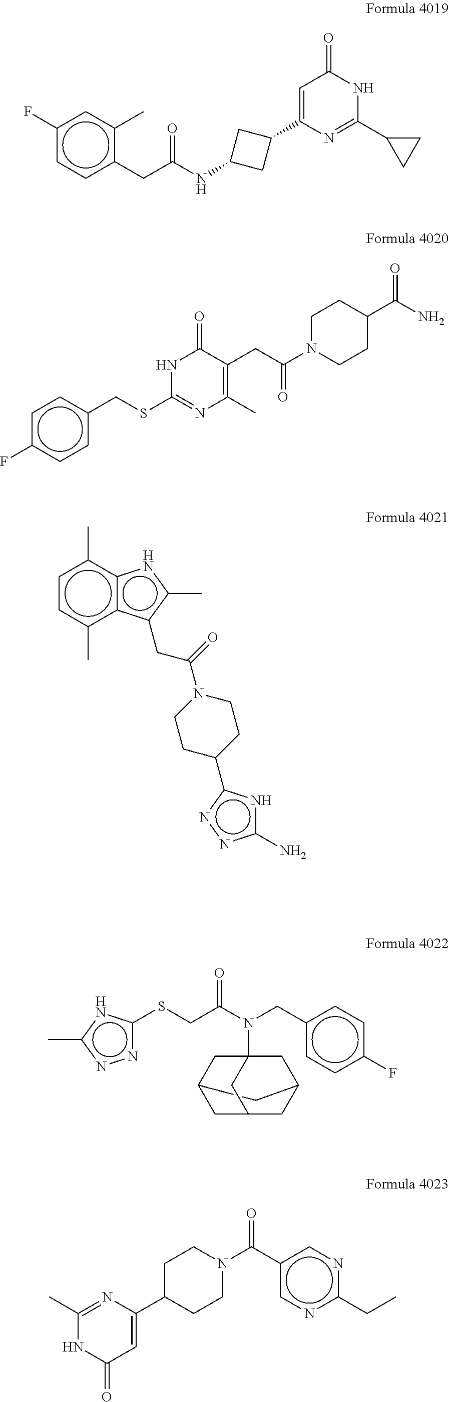

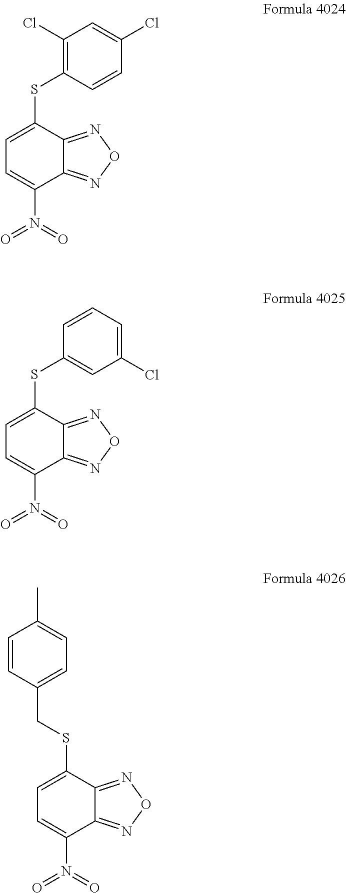 [SMILES: O=[N+]([O-])c1ccc(Sc2ccc(Cl)cc2Cl)c2nonc12 ; O=[N+]([O-])c1ccc(Sc2cccc(Cl)c2)c2nonc12 ; Cc1ccc(CSc2ccc([N+](=O)[O-])c3nonc23)cc1]

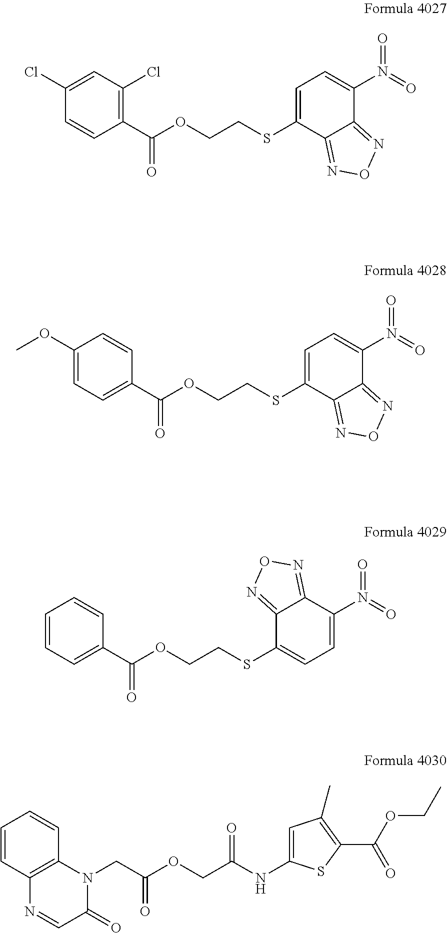 [SMILES: O=C(OCCSc1ccc([N+](=O)[O-])c2nonc12)c1ccc(Cl)cc1Cl ; COc1ccc(C(=O)OCCSc2ccc([N+](=O)[O-])c3nonc23)cc1 ; O=C(OCCSc1ccc([N+](=O)[O-])c2nonc12)c1ccccc1 ; CCOC(=O)c1sc(NC(=O)COC(=O)Cn2c(=O)cnc3ccccc32)cc1C]

Clause 11. The method of clause 1, wherein the compound has a formula selected from any one of Formulas 5001 to 5023:

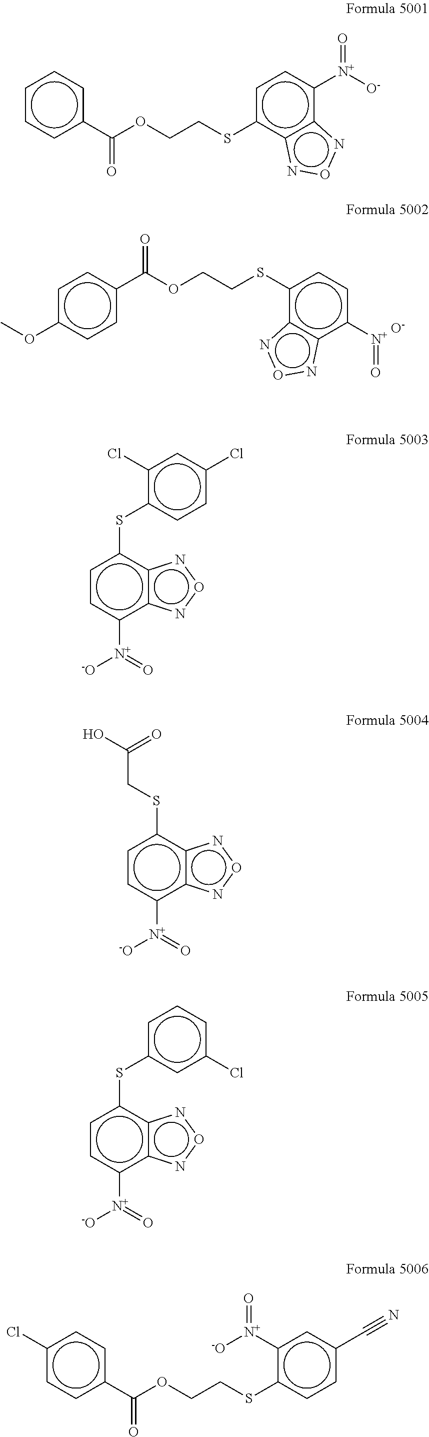

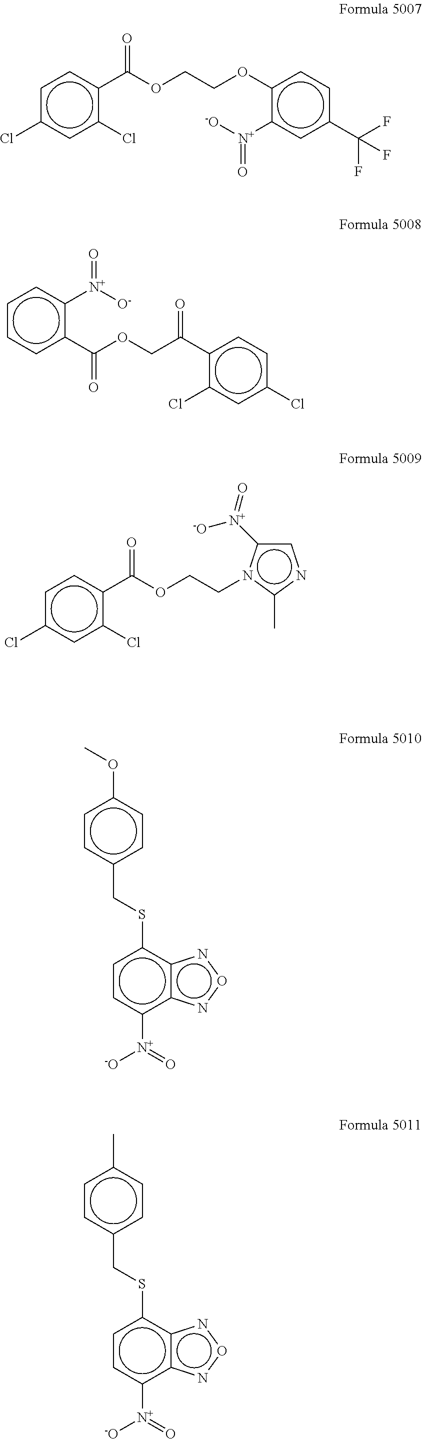

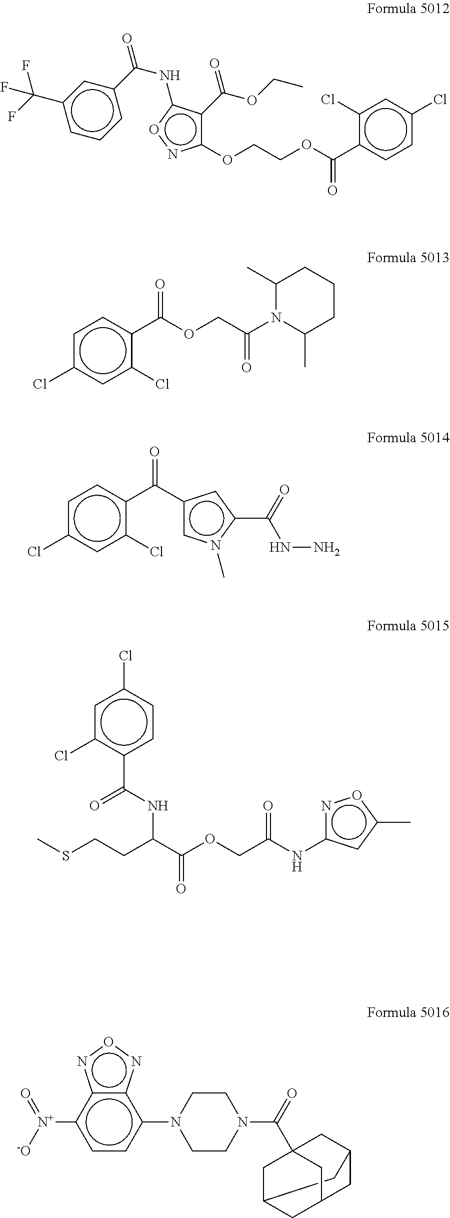

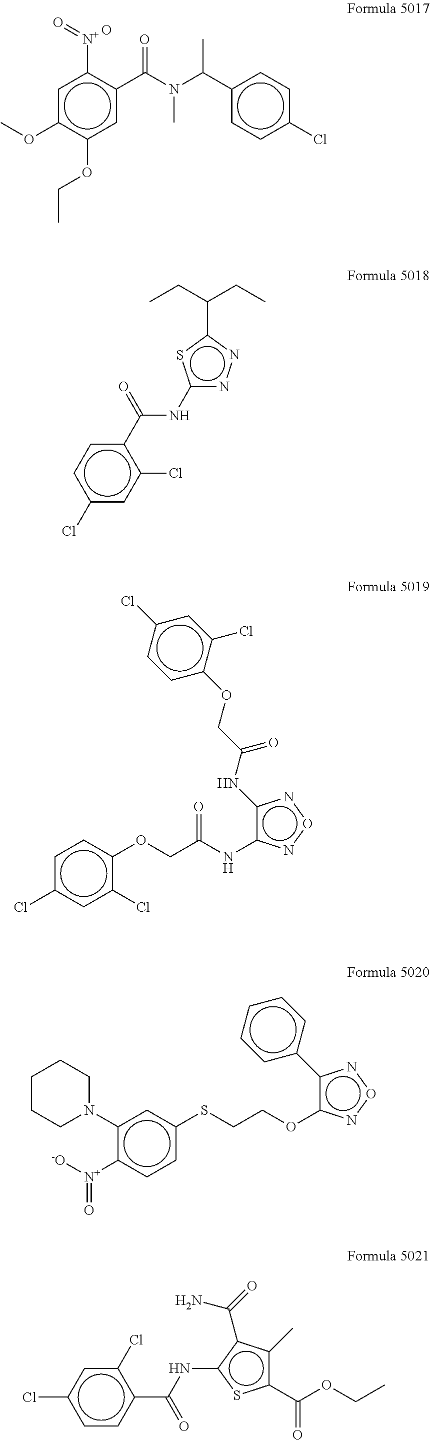

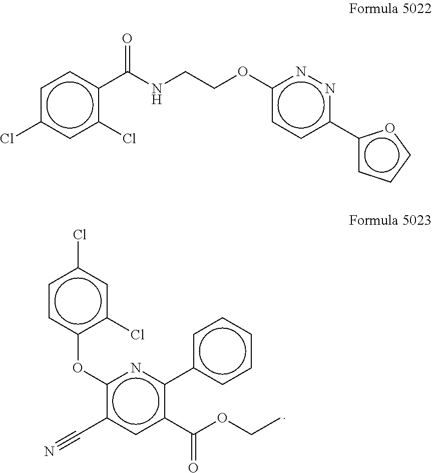

Clause 12. A method of treating or preventing a disease alleviated by inhibiting ANAT in a subject in need thereof, comprising administering to the subject a therapeutically effective amount of a compound of any one of Formulas VIII-a to VIII-e:

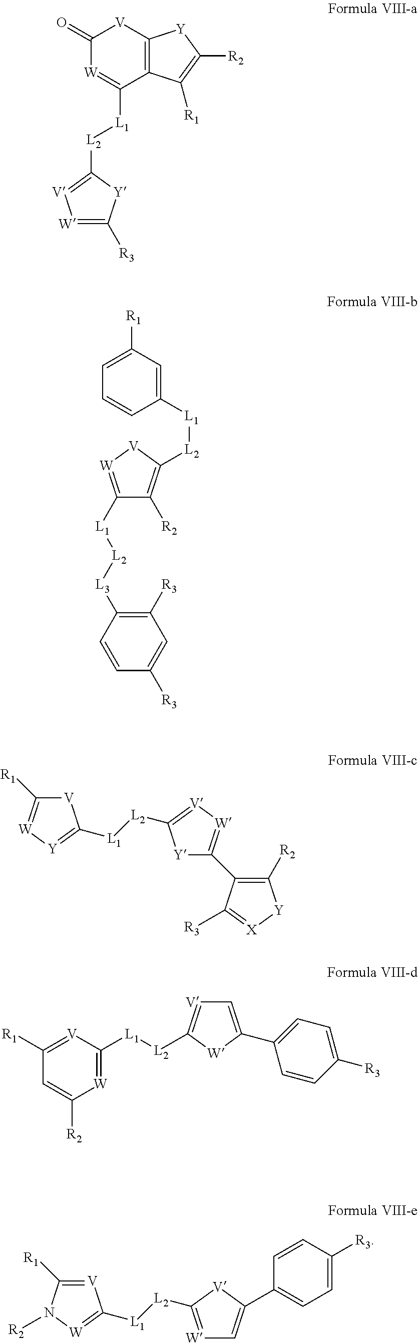

wherein in Formulas VIII-a to VIII-e:

V, W, V′, W′, Y′, X, Y, and Z are independently selected at each occurrence from C, CH, CH₂, N, NH, NR^{a}, O, S

R₁, R₂, and R₃ are independently selected at each occurrence from hydrogen, optionally substituted alkylheteroaryl, optionally substituted alkyl, optionally substituted heteroalkyl, optionally substituted alkenyl, optionally substituted alkynyl, optionally substituted cycloalkyl, optionally substituted heterocycloalkyl, optionally substituted fused heterocycloalkyl, optionally substituted aryl, optionally substituted arylalkyl, optionally substituted heteroaryl, optionally substituted alkylheteroaryl, optionally substituted heteroarylalkyl, optionally substituted alkoxy, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂, wherein R₁, R₂, and R₃ can optionally be joined to form a carbo- or heterocycle;

L₁, L₂, and L₃ are linkers comprising independently at each occurrence one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a}C(O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl;

wherein any optional substituent is independently selected at each occurrence from optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂; and

R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.

Clause 13. The method of clause 12, wherein the compound has a formula selected from any one of Formulas 6001 to 6005:

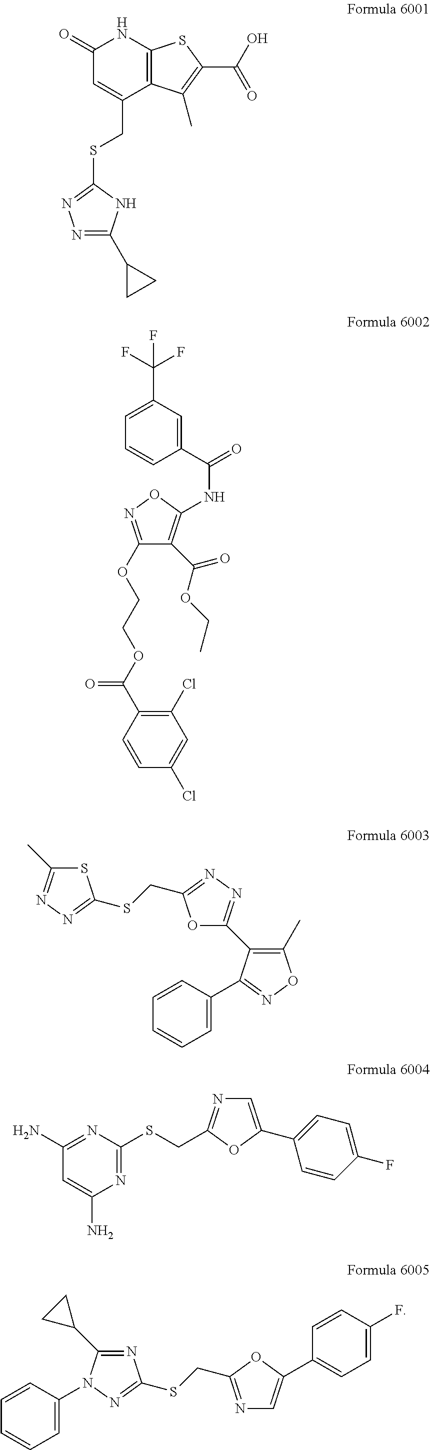

Clause 14. The method of clause 1 or clause 12, wherein the subject has a genetic defect affecting aspartoacylase enzyme activity.

Clause 15. The method of clause 1 or clause 12, wherein the subject has a deficient ability to metabolize N-acetylaspartate.

Clause 16. The method of clause 1 or clause 12, wherein the subject has elevated levels of the brain metabolite N-acetylaspartate (NAA).

Clause 17. The method of any one of clauses 1 to 16, wherein the disease is Canavan disease.

Clause 18. The method of any one of clauses 1 to 16, wherein the disease is cancer.

Clause 19. The method of any one of clauses 1 to 16, wherein the disease is lung cancer.

Clause 20. The method of any one of clauses 1 to 16, wherein the disease is an inborn error of metabolism.

Clause 21. The method of any one of clauses 1 to 16, wherein the disease is epilepsy.

Clause 22. The method of any one of clauses 1 to 16, wherein the disease is leukodystrophy.

Clause 23. The method of any one of clauses 1 to 16, wherein the disease is N-acetyl aspartate deficiency.

Clause 24. The method of any one of clauses 1 to 23, wherein the subject is human.

Clause 25. The method of any one of clauses 1 to 24, wherein the compound is administered in a dosage unit form.

Clause 26. The method of clause 25, wherein the dosage unit comprises a physiologically compatible carrier medium.

Clause 27. A pharmaceutical composition for treating or preventing a disease alleviated by inhibiting ANAT in a subject in need thereof, comprising a compound of Formula IV, Formula V, Formula I, Formula II, Formula III-a, Formula III-b, Formula VI, or Formula VII:

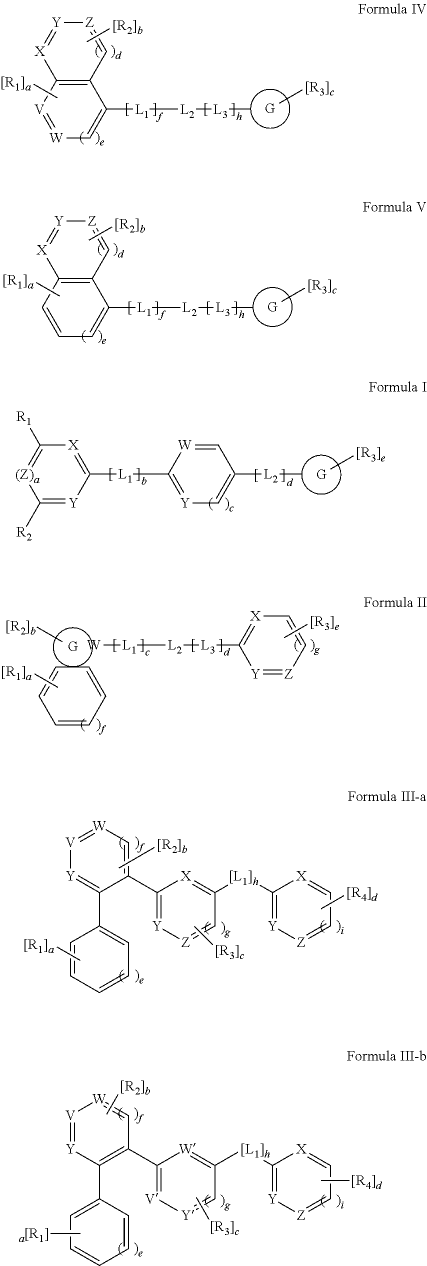

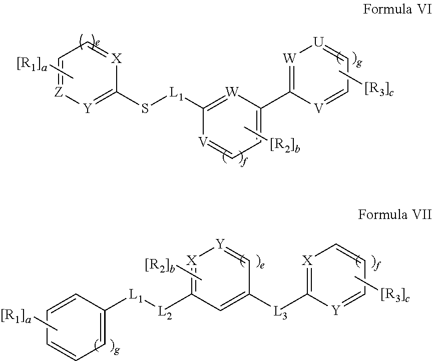

or a pharmaceutically acceptable analog, derivative, salt, solvate, hydrate, cocrystal, or prodrug thereof, wherein in Formulas I to VII:

U, V, W, V′, W′, Y′, X, Y, and Z are independently selected at each occurrence from C, CH, CH₂, N, NH, NR^{a}, O, S

G is independently at each occurrence a mono- or polycyclic optionally substituted cycloalkyl, mono- or polycyclic optionally substituted heterocycloalkyl, mono- or polycyclic optionally substituted aryl, mono- or polycyclic optionally substituted arylalkyl, mono- or polycyclic optionally substituted heteroaryl, and mono- or polycyclic optionally substituted heteroarylalkyl;

R₁, R₂, and R₃ are independently selected at each occurrence from hydrogen, optionally substituted alkylheteroaryl, optionally substituted alkyl, optionally substituted heteroalkyl, optionally substituted alkenyl, optionally substituted alkynyl, optionally substituted cycloalkyl, optionally substituted heterocycloalkyl, optionally substituted fused heterocycloalkyl, optionally substituted aryl, optionally substituted arylalkyl, optionally substituted heteroaryl, optionally substituted alkylheteroaryl, optionally substituted heteroarylalkyl, optionally substituted alkoxy, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂, wherein R₁, R₂, and R₃ can optionally be joined to form a carbo- or heterocycle;

L₁, L₂, and L₃ are linkers comprising independently at each occurrence one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a} C(O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl;

wherein any optional substituent is independently selected at each occurrence from optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂;

R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and

a, b, c, d, e, f, g, h, and i are independently at each occurrence an integer selected from 0, 1, 2, 3, 4, 5, and 6,

wherein the amount of compound in the composition is a therapeutically effective amount for the treatment or prevention of a disease alleviated by inhibiting aspartate N-acetyltransferase (ANAT) activity in a subject in need thereof.

Clause 28. The pharmaceutical composition of clause 27, wherein G is independently at each occurrence selected from benzene, pyridine, pyrimidine, pyrazine, pyridazine, and triazine.

Clause 29. The pharmaceutical composition of clause 27, wherein G is independently at each occurrence selected from furan, thiophene, pyrrole, thiazole, isothiazole, 1,2,3-thiadiazole, imidazole, oxazole, isoxazole, 1,2,3-oxadiazole, 1,3,4-oxadiazole, 1,2,4-oxadiazole, 1,2,3-triazole, 1,2,4-triazole, and pyrazole.

Clause 30. The pharmaceutical composition of clause 27, wherein G is independently at each occurrence selected from naphthalene, quinoline, isoquinoline, cinnoline, quinoxaline, phtalazine, pyridopyrazine, pteridine, pyridopyridazine, naphtyridine, carbazole, dibenzofuran, or quinazoline.

Clause 31. The pharmaceutical composition of clause 27, wherein G is independently at each occurrence selected from indole, benzoxazole, benzothiophene, benzimidazole, indazole, benzotriazole, pyrrolopyridine, triazolopyridine, purine, indolizine, pyrrolopyrimidine, pyrrolopyrazine, pyrrolopyrimine, pyrrolopyridazine, imidazopyridine, pyrazolopyridine, imidazopyridazine, or imidazopyrimidine.

Clause 32. The pharmaceutical composition of clause 27, wherein the compound has a formula selected from any one of Formulas 1 to 5 described herein.

Clause 33. The pharmaceutical composition of clause 27, wherein the compound has a formula selected from any one of Formulas 1001 to 1024 described herein.

Clause 34. The pharmaceutical composition of clause 27, wherein the compound has a formula selected from any one of Formulas 2001 to 2025 described herein.

Clause 35. The pharmaceutical composition of clause 27, wherein the compound has a formula selected from any one of Formulas 3001 to 3023 described herein.

Clause 36. The pharmaceutical composition of clause 27, wherein the compound has a formula selected from any one of Formulas 4001 to 4030 described herein.

Clause 37. The pharmaceutical composition of clause 27, wherein the compound has a formula selected from any one of Formulas 5001 to 5023 described herein.

Clause 38. A pharmaceutical composition for treating or preventing a disease alleviated by inhibiting ANAT in a subject in need thereof, comprising a therapeutically effective amount of a compound of any one of Formulas VIII-a to VIII-e:

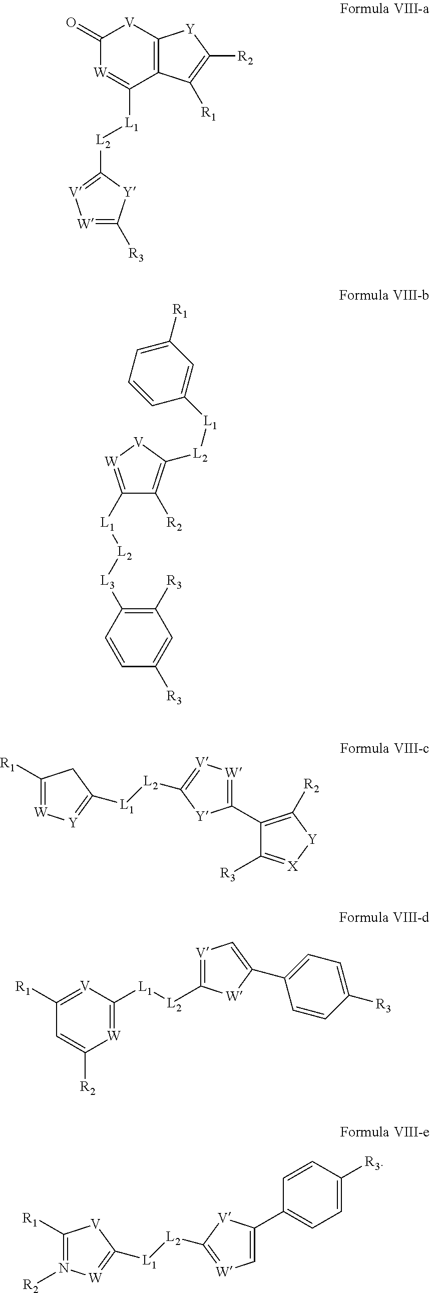

wherein in Formulas VIII-a to VIII-e:

V, W, V′, W′, Y′, X, Y, and Z are independently selected at each occurrence from C, CH, CH₂, N, NH, NR^{a}, O, S

R₁, R₂, and R₃ are independently selected at each occurrence from hydrogen, optionally substituted alkylheteroaryl, optionally substituted alkyl, optionally substituted heteroalkyl, optionally substituted alkenyl, optionally substituted alkynyl, optionally substituted cycloalkyl, optionally substituted heterocycloalkyl, optionally substituted fused heterocycloalkyl, optionally substituted aryl, optionally substituted arylalkyl, optionally substituted heteroaryl, optionally substituted alkylheteroaryl, optionally substituted heteroarylalkyl, optionally substituted alkoxy, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂, wherein R₁, R₂, and R₃ can optionally be joined to form a carbo- or heterocycle;

L₁, L₂, and L₃ are linkers comprising independently at each occurrence one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a}C(O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl;

wherein any optional substituent is independently selected at each occurrence from optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂; and

R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;

wherein the amount of compound in the composition is a therapeutically effective amount for the treatment or prevention of a disease alleviated by inhibiting aspartate N-acetyltransferase (ANAT) activity in a subject in need thereof.

Clause 39. The pharmaceutical composition of clause 38, wherein the compound has a formula selected from any one of Formulas 6001 to 6005 described herein.

Clause 40. The pharmaceutical composition of clause 27 or clause 38, wherein the subject has a genetic defect affecting aspartoacylase enzyme activity.

Clause 41. The pharmaceutical composition of clause 27 or clause 38, wherein the subject has a deficient ability to metabolize N-acetylaspartate.

Clause 42. The pharmaceutical composition of clause 27 or clause 38, wherein the subject has elevated levels of the brain metabolite N-acetylaspartate (NAA).

Clause 43. The pharmaceutical composition of any one of clauses 27 to 42, wherein the disease is Canavan disease.

Clause 44. The pharmaceutical composition of any one of clauses 27 to 42, wherein the disease is cancer.

Clause 45. The pharmaceutical composition of any one of clauses 27 to 42, wherein the disease is lung cancer.

Clause 46. The pharmaceutical composition of any one of clauses 27 to 42, wherein the disease is an inborn error of metabolism.

Clause 47. The pharmaceutical composition of any one of clauses 27 to 42, wherein the disease is epilepsy.

Clause 48. The pharmaceutical composition of any one of clauses 27 to 42, wherein the disease is leukodystrophy.

Clause 49. The pharmaceutical composition of any one of clauses 27 to 42, wherein the disease is N-acetyl aspartate deficiency.

Clause 50. The pharmaceutical composition of any one of clauses 27 to 49, wherein the subject is human.

Clause 51. The pharmaceutical composition of any one of clauses 27 to 50, wherein the compound is administered in a dosage unit form.

Clause 52. The pharmaceutical composition of clause 51, wherein the dosage unit comprises a physiologically compatible carrier medium.

Clause 53. A compound of Formula IV, Formula V, Formula I, Formula II, Formula III-a, Formula III-b, Formula VI, or Formula VII:

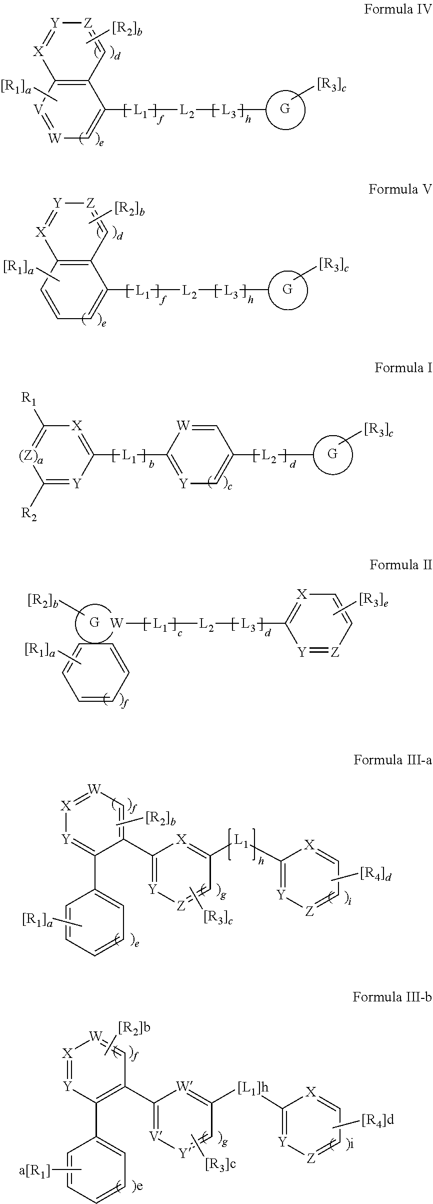

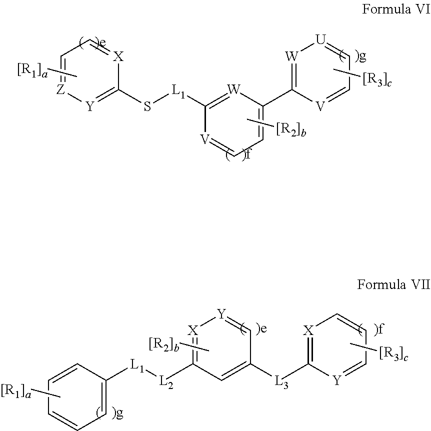

or a pharmaceutically acceptable analog, derivative, salt, solvate, hydrate, cocrystal, or prodrug thereof, wherein in Formulas I to VII:

U, V, W, V′, W′, Y′, X, Y, and Z are independently selected at each occurrence from C, CH, CH₂, N, NH, NR^{a}, O, S

G is independently at each occurrence a mono- or polycyclic optionally substituted cycloalkyl, mono- or polycyclic optionally substituted heterocycloalkyl, mono- or polycyclic optionally substituted aryl, mono- or polycyclic optionally substituted arylalkyl, mono- or polycyclic optionally substituted heteroaryl, and mono- or polycyclic optionally substituted heteroarylalkyl;

R₁, R₂, and R₃ are independently selected at each occurrence from hydrogen, optionally substituted alkylheteroaryl, optionally substituted alkyl, optionally substituted heteroalkyl, optionally substituted alkenyl, optionally substituted alkynyl, optionally substituted cycloalkyl, optionally substituted heterocycloalkyl, optionally substituted fused heterocycloalkyl, optionally substituted aryl, optionally substituted arylalkyl, optionally substituted heteroaryl, optionally substituted alkylheteroaryl, optionally substituted heteroarylalkyl, optionally substituted alkoxy, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂, wherein R₁, R₂, and R₃ can optionally be joined to form a carbo- or heterocycle;

L₁, L₂, and L₃ are linkers comprising independently at each occurrence one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a}C(O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl; wherein any optional substituent is independently selected at each occurrence from optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂;

R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and

a, b, c, d, e, f, g, h, and i are independently at each occurrence an integer selected from 0, 1, 2, 3, 4, 5, and 6.

Clause 54. The compound of clause 53, wherein G is independently at each occurrence selected from benzene, pyridine, pyrimidine, pyrazine, pyridazine, and triazine.

Clause 55. The compound of clause 53, wherein G is independently at each occurrence selected from furan, thiophene, pyrrole, thiazole, isothiazole, 1,2,3-thiadiazole, imidazole, oxazole, isoxazole, 1,2,3-oxadiazole, 1,3,4-oxadiazole, 1,2,4-oxadiazole, 1,2,3-triazole, 1,2,4-triazole, and pyrazole.

Clause 56. The compound of clause 53, wherein G is independently at each occurrence selected from naphthalene, quinoline, isoquinoline, cinnoline, quinoxaline, phtalazine, pyridopyrazine, pteridine, pyridopyridazine, naphtyridine, carbazole, dibenzofuran, or quinazoline.

Clause 57. The compound of clause 53, wherein G is independently at each occurrence selected from indole, benzoxazole, benzothiophene, benzimidazole, indazole, benzotriazole, pyrrolopyridine, triazolopyridine, purine, indolizine, pyrrolopyrimidine, pyrrolopyrazine, pyrrolopyrimine, pyrrolopyridazine, imidazopyridine, pyrazolopyridine, imidazopyridazine, or imidazopyrimidine.

Clause 58. The compound of clause 53, wherein the compound has a formula selected from any one of Formulas 1 to 5 described herein.

Clause 59. The compound of clause 53, wherein the compound has a formula selected from any one of Formulas 1001 to 1024 described herein.

Clause 60. The compound of clause 53, wherein the compound has a formula selected from any one of Formulas 2001 to 2025 described herein.

Clause 61. The compound of clause 53, wherein the compound has a formula selected from any one of Formulas 3001 to 3023 described herein.

Clause 62. The compound of clause 53, wherein the compound has a formula selected from any one of Formulas 4001 to 4030 described herein.

Clause 63. The compound of clause 53, wherein the compound has a formula selected from any one of Formulas 5001 to 5023 described herein.

Clause 64. A compound of any one of Formulas VIII-a to VIII-e:

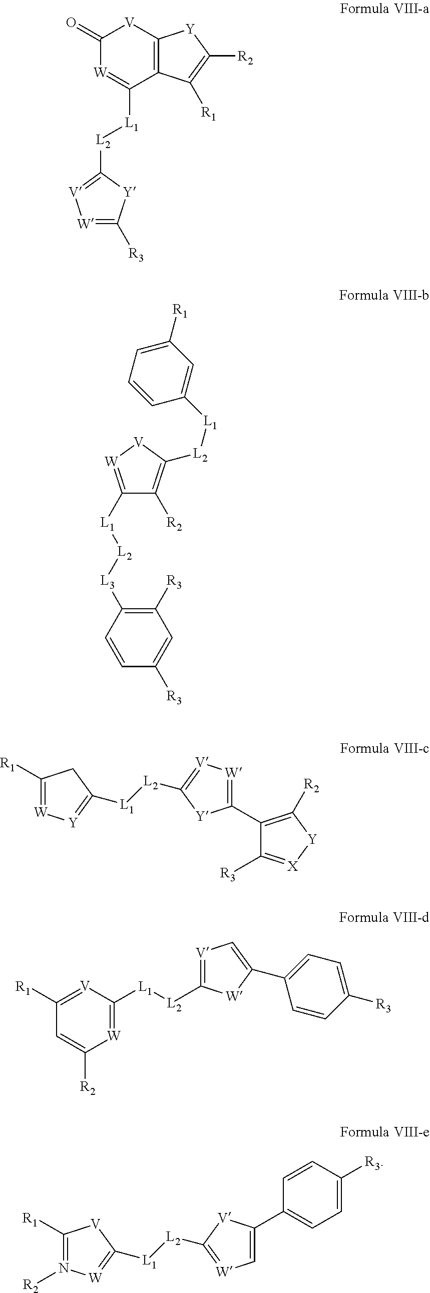

wherein in Formulas VIII-a to VIII-e:

V, W, V′, W′, Y′, X, Y, and Z are independently selected at each occurrence from C, CH, CH₂, N, NH, NR^{a}, O, S

R₁, R₂, and R₃ are independently selected at each occurrence from hydrogen, optionally substituted alkylheteroaryl, optionally substituted alkyl, optionally substituted heteroalkyl, optionally substituted alkenyl, optionally substituted alkynyl, optionally substituted cycloalkyl, optionally substituted heterocycloalkyl, optionally substituted fused heterocycloalkyl, optionally substituted aryl, optionally substituted arylalkyl, optionally substituted heteroaryl, optionally substituted alkylheteroaryl, optionally substituted heteroarylalkyl, optionally substituted alkoxy, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂, wherein R₁, R₂, and R₃ can optionally be joined to form a carbo- or heterocycle;

L₁, L₂, and L₃ are linkers comprising independently at each occurrence one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a}C(O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl;

wherein any optional substituent is independently selected at each occurrence from optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂; and

R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.

Clause 65. The compound of clause 64, wherein the compound has a formula selected from any one of Formulas 6001 to 6005 described herein.

Clause 66. The compound of any one of clauses 53 to 65, wherein the compound is an aspartate N-acetyltransferase (ANAT) inhibitor.

Clause 67. The compound of any one of clauses 53 to 66, wherein the compound has a therapeutic effect in the treatment or prevention of a disease alleviated by inhibiting aspartate N-acetyltransferase (ANAT) activity in a subject in need thereof.

Clause 68. The compound of clause 67, wherein the subject has a genetic defect affecting aspartoacylase enzyme activity.

Clause 69. The compound of clause 67, wherein the subject has a deficient ability to metabolize N-acetylaspartate.

Clause 70. The compound of clause 67, wherein the subject has elevated levels of the brain metabolite N-acetylaspartate (NAA).

Clause 71. The compound of clause 67, wherein the disease is Canavan disease.

Clause 72. The compound of clause 67, wherein the disease is cancer.

Clause 73. The compound of clause 67, wherein the disease is lung cancer.

Clause 74. The compound of clause 67, wherein the disease is an inborn error of metabolism.

Clause 75. The compound of clause 67, wherein the disease is epilepsy.

Clause 76. The compound of clause 67, wherein the disease is leukodystrophy.

Clause 77. The compound of clause 67, wherein the disease is N-acetyl aspartate deficiency.

Clause 78. The compound of clause 67, wherein the subject is human.

### Examples

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

AtomNet is the first deep neural network for structure-based drug discovery. A single global AtomNet model is trained to predict binding affinity using several million small molecule Kᵢ and IC₅₀ values, and several thousand protein structures of different families. This model can then be used prospectively, even against novel binding sites with no known ligands, while the use of a single global model helps to prevent overfitting.

Training proceeds as follows. First, the binding site on a given protein structure is defined using a flooding algorithm based on an initial seed. The initial starting point of the flooding algorithm may be determined from a variety of methods, including a bound ligand annotated in the RCSB PDB database, crucial residues as revealed by mutagenesis studies, or identification of catalytic motifs previously reported in the literature. Second, the coordinates of the co-complex are shifted to a three-dimensional Cartesian system with an origin at the center-of-mass of the binding site. Data augmentation is then performed by randomly rotating and translating the protein around the binding site's center of mass. This prevents the neural network from learning a preferred protein orientation; as gravity rarely makes a significant contribution to binding, any such uncovered signal would be an inadvertent artifact from our data curation process. Third, for a given ligand, multiple poses are sampled within the binding site cavity. Each of these poses represents a putative co-complex and therefore, unlike previous structure-based predictive methods such as docking, experimental co-complexes for either training or prediction are not required. While this may introduce errors from inaccurate ligand poses, state-of-the-art pose samplers have become sufficiently accurate while the generation of predicted co-complexes enables the use of binding assay data for which there are no experimental co-complex structures.

Each generated co-complex is then rasterized into a fixed-size regular three-dimensional grid, where the values at each grid point represent the structural features that are present at each grid point. Analogously to a photo pixel containing three separate channels representing the presence of red, green, and blue colors, the grid points represent the presence of different atom types (or more complex protein-ligand descriptors such as SPLIF, SIFt, or APIF). These grids serve as the input to a convolutional neural network, defining the network's receptive field. A network architecture of a 30×30×30 grid with a 1 Å spacing for the input layer is used, followed by five convolutional layers of 32×3\hat{ }3, 64×3\hat{ }3, 64×3\hat{ }3, 64×3\hat{ }3, 64×2\hat{ }3 (number of filters x filer-dimension), and a fully-connected layer with 256 ReLU hidden units. The scores for each pose in the ensemble are then combined through a weighted Boltzmann averaging to produce a final score. These scores are then compared against the experimentally-measured Kᵢ or IC₅₀ of the protein and ligand pair, and the weights of the neural network are adjusted to reduce the error between the predicted and experimentally-measured affinity using a mean-square-error loss function. Training is done using the ADAM adaptive learning method, the backpropagation algorithm, and mini-batches of 64 examples per gradient step.

Prediction follows an analogous process. An orthosteric or allosteric binding site on the target protein is selected. Next, for each molecule in a given molecular screening library of interest, a set of poses within the binding site is generated. Each of these are scored by the trained model, and the list of molecules is ranked by their scores. A set of molecules on the top of the list were then purchased for experimental testing, subject only to supplier availability and price. Importantly, a manual bias through a visual inspection of the compounds was not introduced. Therefore, this process yields an actual assessment of the ability of the software to identify hits with minimal operator intuition.

Given the challenges of studying membrane-associated enzymes, at the time the project was initiated, there were no publicly available structures of the human ANAT protein. A search for template structures using SWISS-MODEL (citation) indicated a number of homologous structures in the sequence identity range of 3% (PDB ID 3N7Z) to 33% (PDB ID 1U6M). After a thorough analysis considering factors such as resolution, alignment coverage, presence of either coenzyme A or acetyl coenzyme A, the list was narrowed to include the putative Bacillus anthracis streptothricin acetyltransferase (PDB ID 3PP9, sequence identity 25%), the putative Agrobacterium fabrum acetyltransferase (PDB ID 2GE3, 25%), and Streptomyces hygroscopicus phosphinothricin N-acetyltransferase (PDB ID 5T7E, 20%) (FIG. 1B). Ultimately, PDB ID 5T7E was selected as the template for comparative structure modeling due to the presence of the substrate in the structure and the similarity of the catalytic activity of the two proteins.

Like the reactions catalyzed by many of these distantly homologous proteins, the human aspartate N-acetyltransferase relies on acetyl coenzyme A as the acetyl group donor to perform its catalytic function (FIGS. 1A, 1B). The catalytic domain of the enzyme is predicted to assume a common Gcn5-related N-acetyltransferase (GNAT) fold. The target-template sequence alignment was constructed using PROMALS3D. The comparative structure model was built using the Full Model Builder module with the Full refinement routine within Molsoft ICM 3.8, retaining the coenzyme A molecule as a rigid body. The model was manually inspected to ensure there were no missing side-chain atoms, hydrogens were added, and the molecule of coenzyme A was replaced with the acetyl coenzyme A from PDB ID 5T7D. The molecule of L-phosphinothricin was used to initiate the flooding algorithm for the definition of the screening site. Despite evidence that many GNAT members exist as dimers, the screen was performed solely against the ANAT protomer due to the added uncertainty of the location of a potential dimerization interface.

Computation affords the ability to screen remarkably large compound collections. Therefore, the screening strategy was designed to be as comprehensive as possible, under reasonable constraints of experimental efficiency and potential for efficacy. The screening library included commercially available compounds from a chemical vendor. Salts were stripped, structures were canonicalized, and duplicates removed. Filtration was also performed to filter out molecules outside of the 100 and 800 Dalton range, those containing more than 7 chiral centers, and those with more than 16 rotational bonds.

The remaining 7,170,625 molecules were scored by AtomNet against the target on an elastic hybrid cluster consisting of GPU and CPU instances on the Amazon Web Service infrastructure. As mentioned above, the AtomNet model used for the hit identification was a global model that was not target specific. Neither the target nor the previously identified inhibitor series were used in training. Further, no homologous (high >40% sequence identity) targets with annotated compound pairs similar to the identified hits were present in the training set. No structural information or pose evaluation was taken into account in the post-filtering process.

The top-scoring compounds were clustered for diversity using the ECFP4 Tanimoto coefficient similarity cutoff of 0.3. The 63 predicted high-affinity binder cluster representatives were purchased for experimental validation.

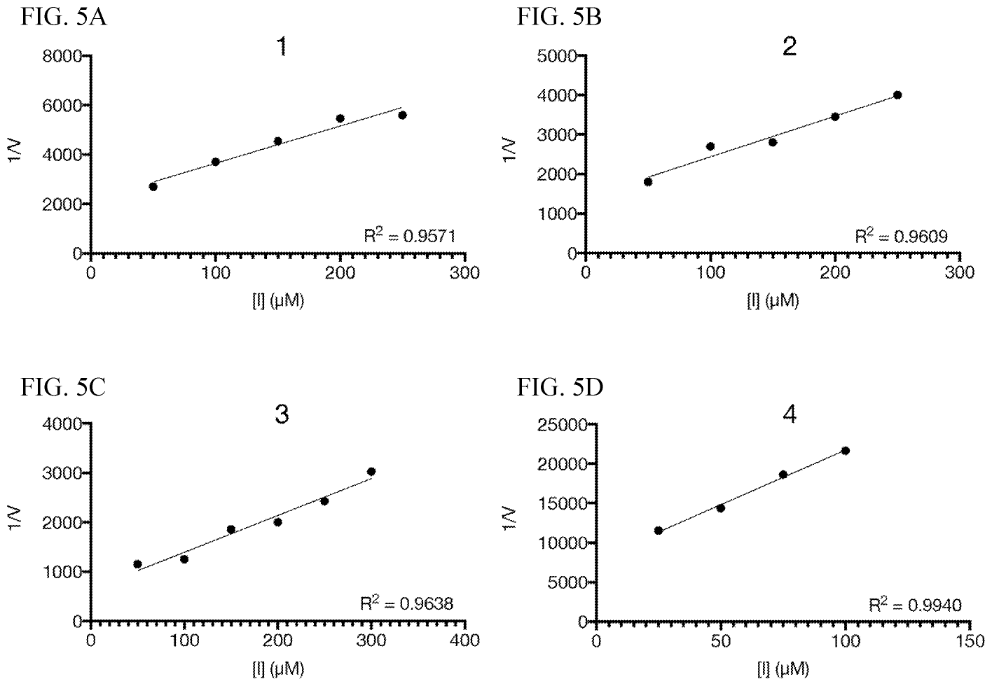

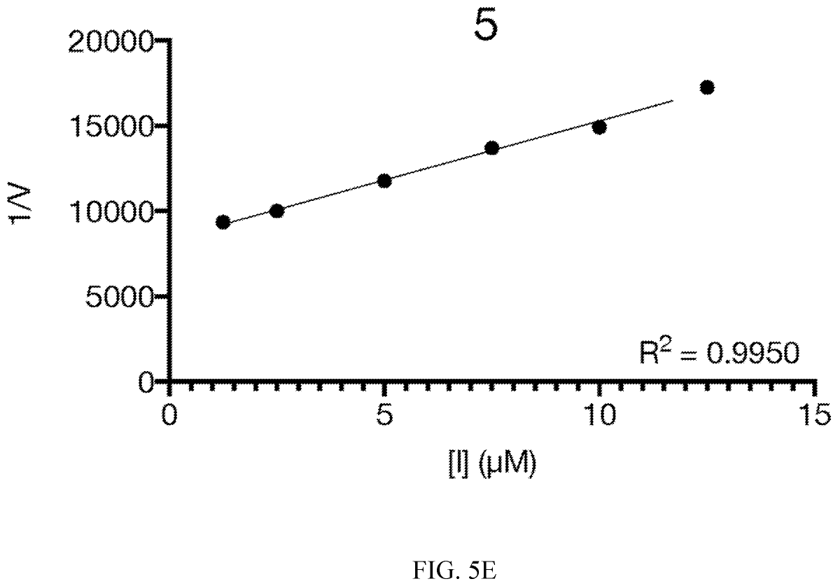

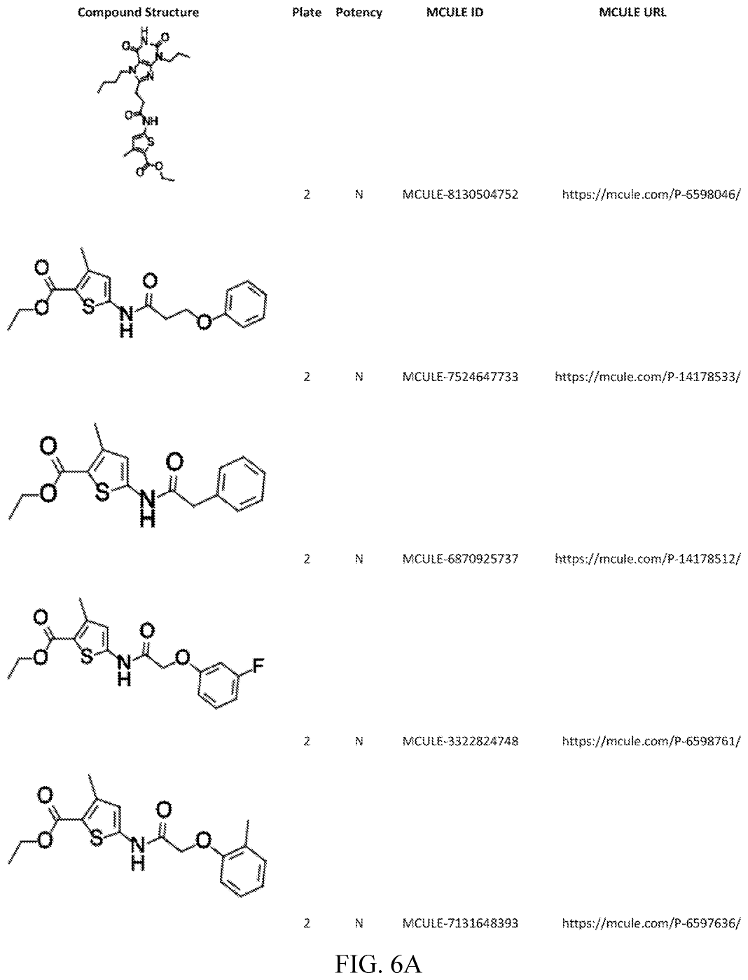

The experimental assay was based on a custom protocol developed by the Viola lab described previously (see, e.g., Thangavelu et al., 2017, “Design and optimization of aspartate N-acetyltransferase inhibitors for the potential treatment of Canavan disease,” Bioorganic & Medicinal Chemistry, 25(3), pp. 870-885). Each of the purchased compounds was tested in an initial screen at 1 mM, followed by a second screen of the initial inhibitors at 50 μM. The compounds which showed inhibition at 50 μM were subsequently evaluated in a dose-response protocol. Of these, five compounds exhibited a Kᵢ value in the low micromolar range (see, FIG. 5 and Table 1).

TABLE 1
Molecular properties of hit molecules. All values calculated using Molsoft ICM 3.8

| Compound ID | Ki (μM) | MW (Da) | logP | HBD | HBA | RB | PSA (Å²) |
|---|---|---|---|---|---|---|---|
| Lipinski values | low | <500 | <5 | <15 | <10 | <10 | <140 |
| 1 | 8.2 | 317 | 2.8 | 4 | 5 | 4 | 76 |
| 2 | 8.5 | 429 | 2.4 | 1 | 11 | 10 | 90 |
| 3 | 9.1 | 371 | 2.8 | 0 | 8 | 5 | 75 |
| 4 | 4.5 | 362 | 2.3 | 3 | 8 | 5 | 86 |
| 5 | 0.91 | 413 | 4.7 | 0 | 11 | 7 | 91 |
| P59H | 0.61 | 469 | 2.8 | 4 | 13 | 10 | 118 |

For further details on Lipinski values, see, e.g., Lipinski, et al. (2001) Adv. Drug Deliv. Rev. 46, 3-26.

In this study, a set of five novel, diverse scaffolds with low-micromolar potency against aspartate N-acetyltransferase was identified. These compounds hold potential as starting points for future therapeutic discovery programs. The resources utilized in the completion of this specific study fall well within the grasp of patient advocacy and academic research communities for application to an expanding number of orphan diseases, which currently suffer from a lack of therapeutic options. The utility of AtomNet, the world's first deep neural network for structure-based drug discovery, has been shown to be high even in this case, where there were no crystal structures and no known drug-like inhibitors to serve as starting points for optimization. Further work is needed to understand the details of the mode of inhibition of these compounds. However, these preliminary results serve as an important case study of prospective discovery and the tremendous promise that novel ML methods hold to catalyze early discovery for even the rarest of diseases.

### Methods

Purification of ANAT

The challenge of solubilizing and purifying the membrane-associated enzyme ANAT has been addressed through the creation of a fusion construct with maltose binding protein (MBP) as a fusion partner, as described previously. Briefly, aspartate ANAT was expressed with an N-terminal MBP tag in E. coli BL21(DE3) cells. A 6×His-tag was inserted at the C-terminal of the enzyme to facilitate purification. 20 ml LB media was grown overnight from a single colony in the presence of kanamycin at a final concentration of 50 μg/ml at 37° C. The overnight culture was then diluted into 2 L LB media and grown with shaking at 37° C. until the OD₆₀₀ reached 0.6. Isopropyl β-D-thiogalactopyranoside (IPTG) was then added at a final concentration of 0.1 mM for induction, and cell growth continued at 16° C. for 16-20 hours. The cell culture was harvested by centrifugation at 11,000 rpm for 15 min. The cells were then resuspended in binding buffer consisting of 20 mM potassium phosphate, pH 7.4, 300 mM NaCl, 20 mM imidazole, and 10% glycerol. The cells were lysed by adding lysozyme (50 μg/ml of buffer) and DNAse (5 μg/ml of buffer) in an ultrasonic dismembrator, followed by clarification of the cells by centrifugation at 11,000 rpm to remove the insoluble cell debris. The clarified supernatant was loaded onto a Ni-NTA column and eluted with a 20-300 mM imidazole gradient in the binding buffer using an AKTA chromatography system. The fractions containing active enzyme purified from the Ni-NTA column were pooled and loaded onto a dextrin-Sepharose column for further purification. The MBP-ANAT fusion enzyme was eluted from this column by using a 0-10 mM maltose gradient. SDS-PAGE gel was run to confirm the molecular weight and purity of the enzyme.

Screening Compound Sourcing

Compounds were purchased from MCULE (mcule.com). The product details for the five hit compounds are as follows:

2-{[5-(p-Fluorophenyl)-1,3-oxazol-2-yl]methylthio}-4,6-pyrimidinediamine (Formula 1). Purchased as vendor ID MCULE-6867206395, product ID P-32058317, batch ID PR-25609.

Ethyl 3-methyl-5-{2-[(2-oxo-1H-quinoxalin-1-yl)acetoxy]acetylamino}-2-thenoate (Formula 2). Purchased as vendor ID MCULE-5685536797, product ID P-10932293, batch ID PR-25647.

5-(5-Methyl-3-phenyl-4-isoxazolyl)-2-[(5-methyl-1,3,4-thiadiazol-2-ylthio)methyl]-1,3,4-oxadiazole (Formula 3). Purchased as vendor ID MCULE-5663978183, product ID P-18034504, batch ID PR-25567.

4-[(5-Cyclopropyl-4H-1,2,4-triazol-3-ylthio)methyl]-3-methyl-6-oxo-1-thia-7-aza-7H-indene-2-carboxylic acid (Formula 4). Purchased as vendor ID MCULE-2601511115, product ID P-32100333, batch ID PR-25608.

2-(7-Nitro-2,1,3-benzofurazan-4-ylthio)ethyl 2,4-dichlorobenzoate (Formula 5). Vendor ID MCULE-2879769428, product ID P-35226327, batch ID PR-25553.

1 mg of each compound was ordered and dissolved in DMSO to a concentration of 10 mM. The LC/MS data for each of the compounds is available in the supplement.

In Vitro Assay

ANAT activity was measured by monitoring the production of coenzyme A using 5,5′-dithio-bis-[2-nitrobenzoic acid] (DTNB). The assay buffer contained 20 mM HEPES, pH 7.5, 150 mM NaCl, 5% glycerol, 40 μM DTNB, 40 μM acetyl-CoA (Kₘ=3.1 μM), 2 mM L-aspartate (Kₘ=0.16 mM), 10 μg of enzyme (k_{cat}=0.071 U/mg), and varying concentrations of inhibitors. The increasing absorption at 412 nm was monitored for up to 30 min during the reaction. 10% DMSO was maintained in the first screen while 20% DMSO was used in the second screen to dissolve the insoluble or partially soluble compounds in the assay wells.

Kᵢ was calculated using the competitive inhibition routine within the nonlinear regression module of GraphPad Prism 8.3.0 with the following equation:

$${K}_{m , obs} = {K}_{m} ( 1 + \frac{[ I ]}{{K}_{i}} ) \\ V = {V}_{max} ( \frac{[ S ]}{{K}_{m , obs} + [ S ]} )$$

where Kₘ for L-aspartate was set to 160 μM, and [I] and [S] denote the concentrations of the inhibitor and substrate, respectively.

Abbreviations: CCR2, CC chemokine receptor 2; CCL2, CC chemokine ligand 2; CCR5, CC chemokine receptor 5; TLC, thin layer chromatography.

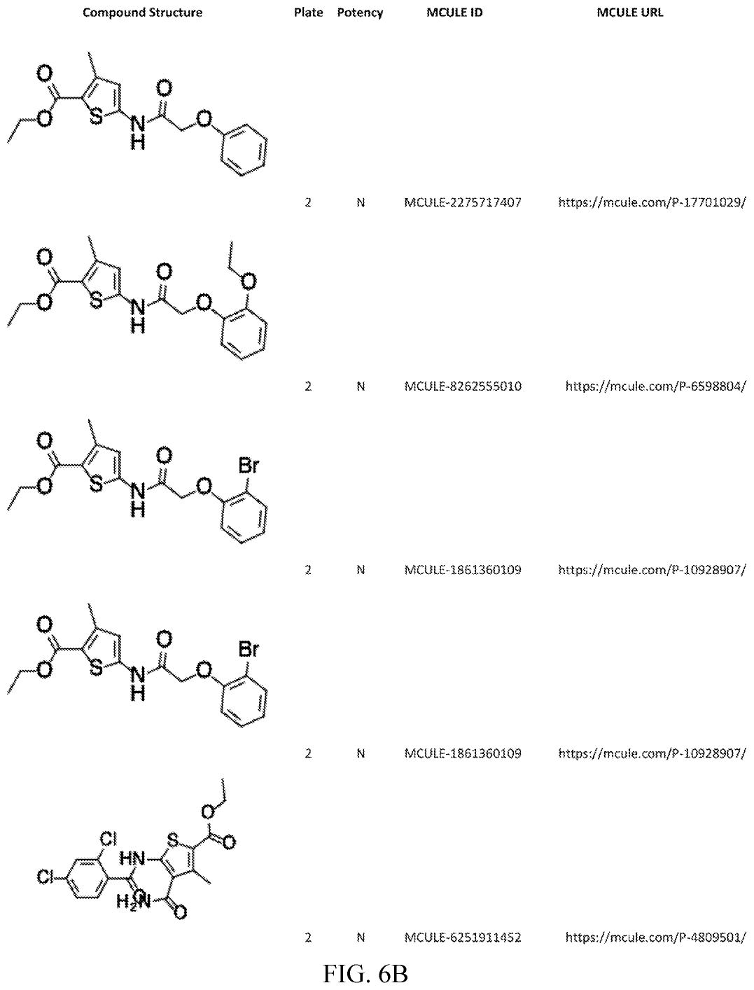

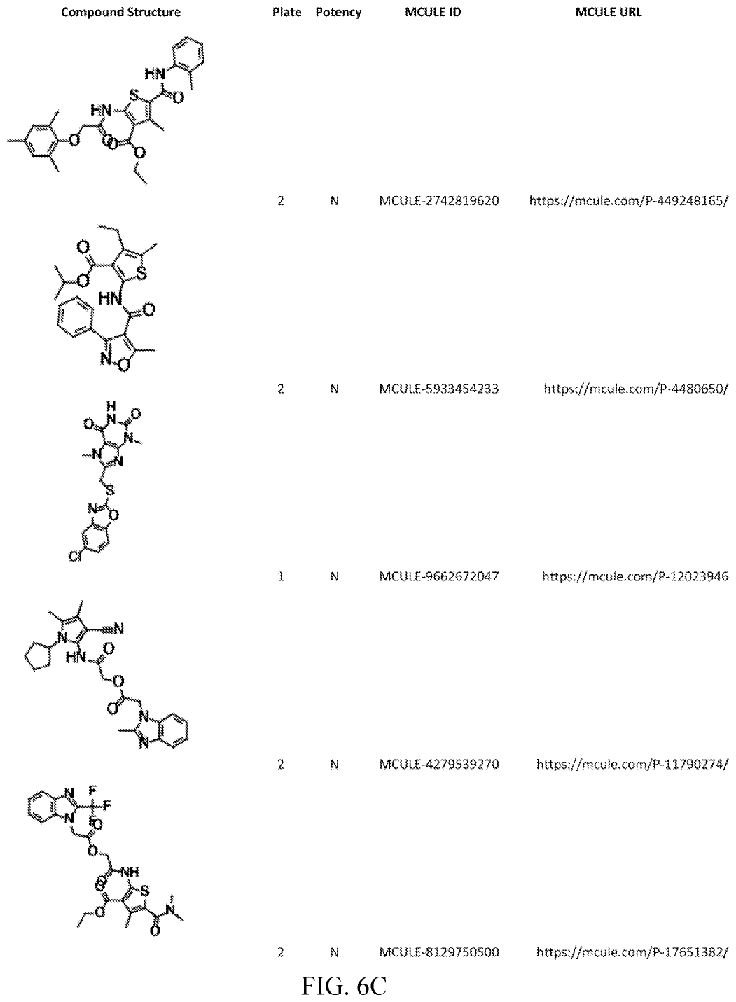

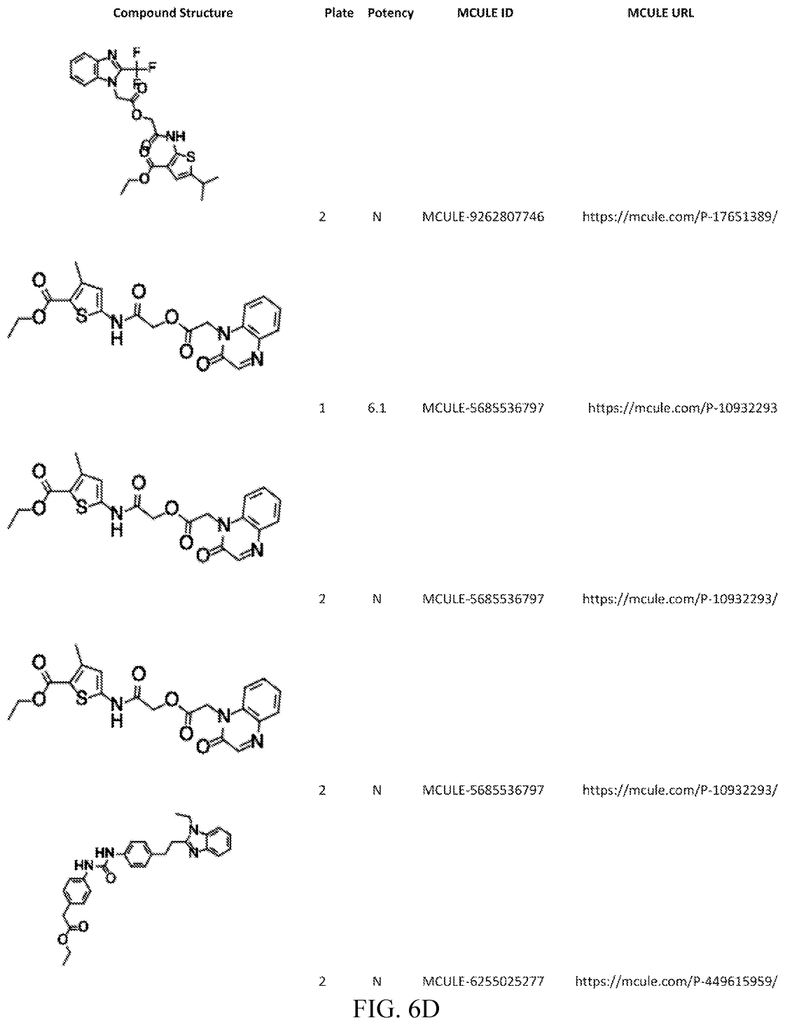

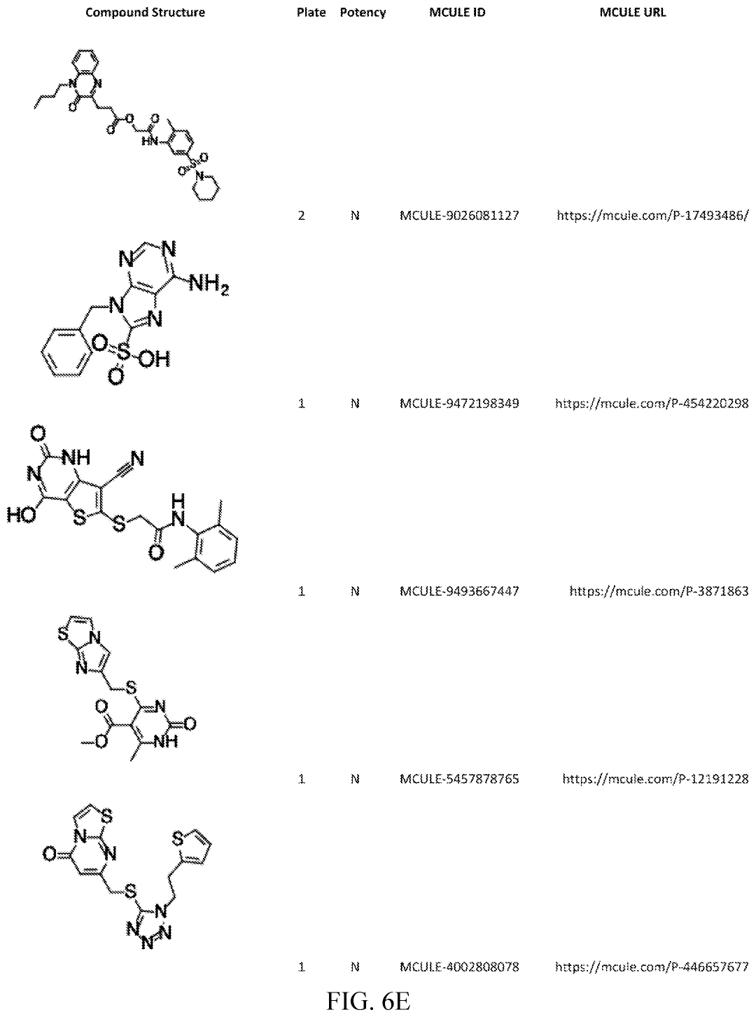

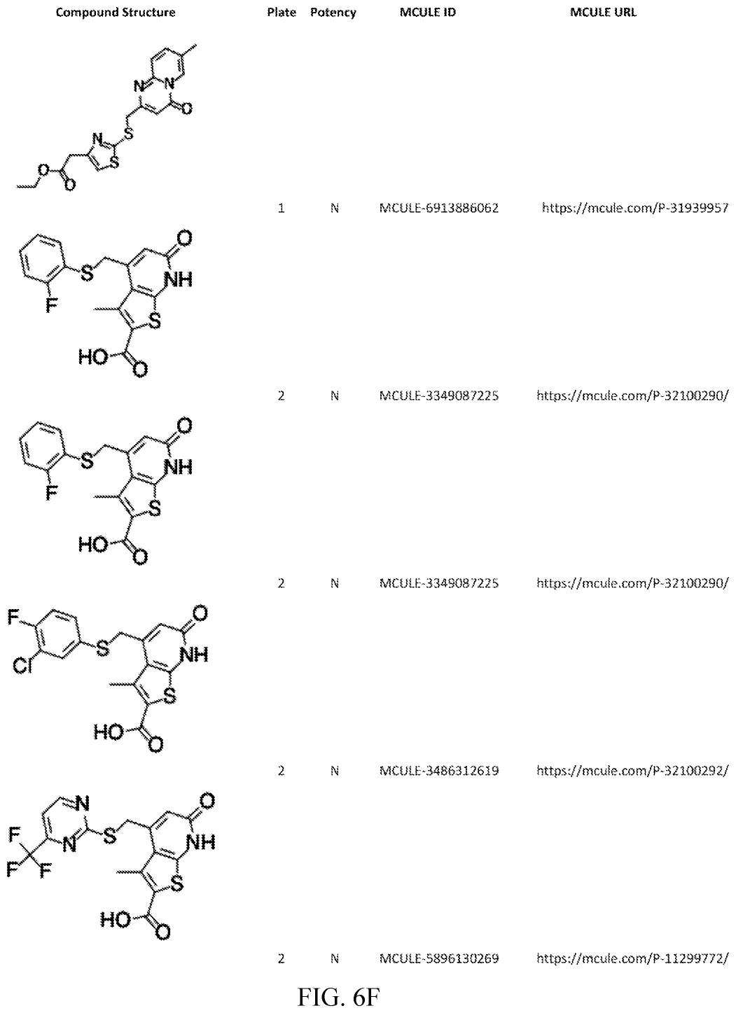

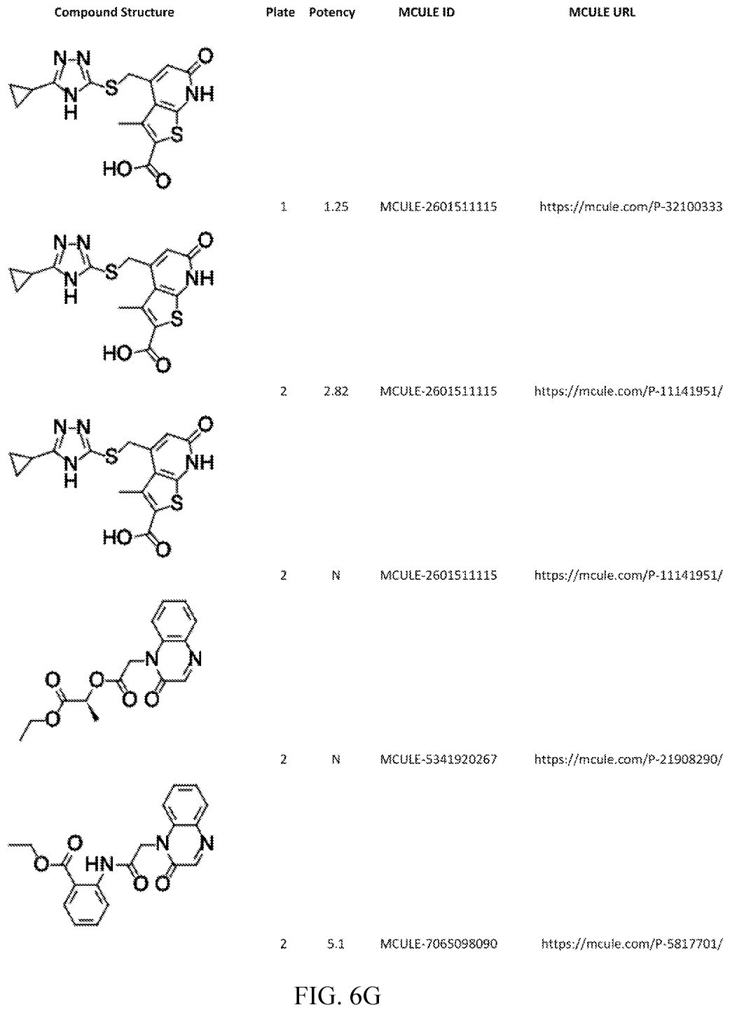

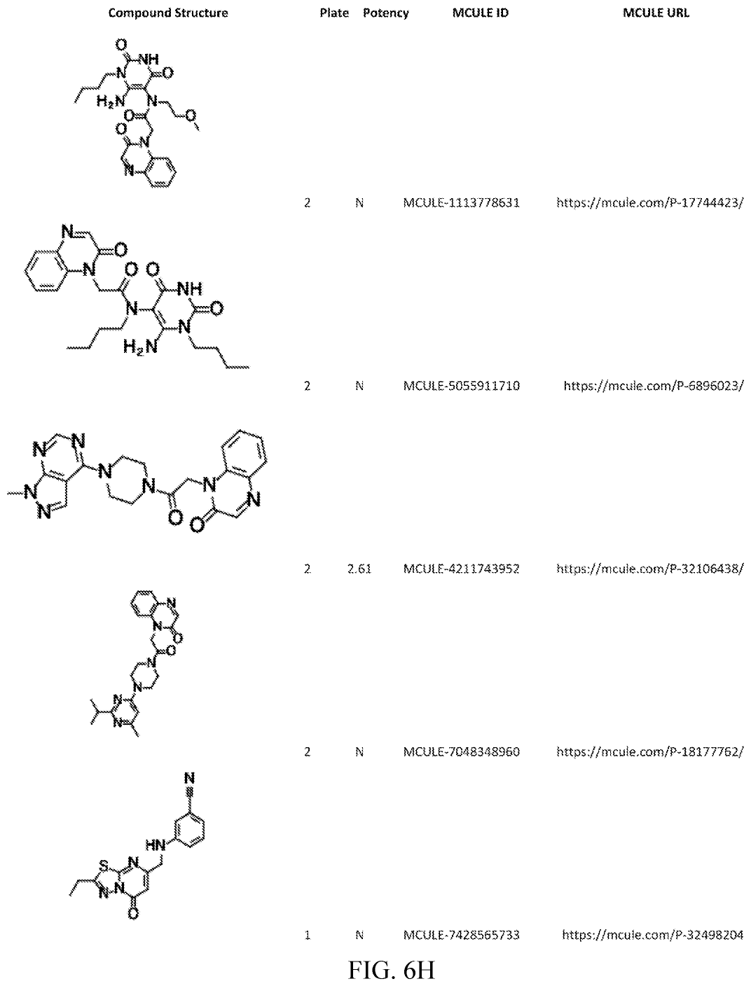

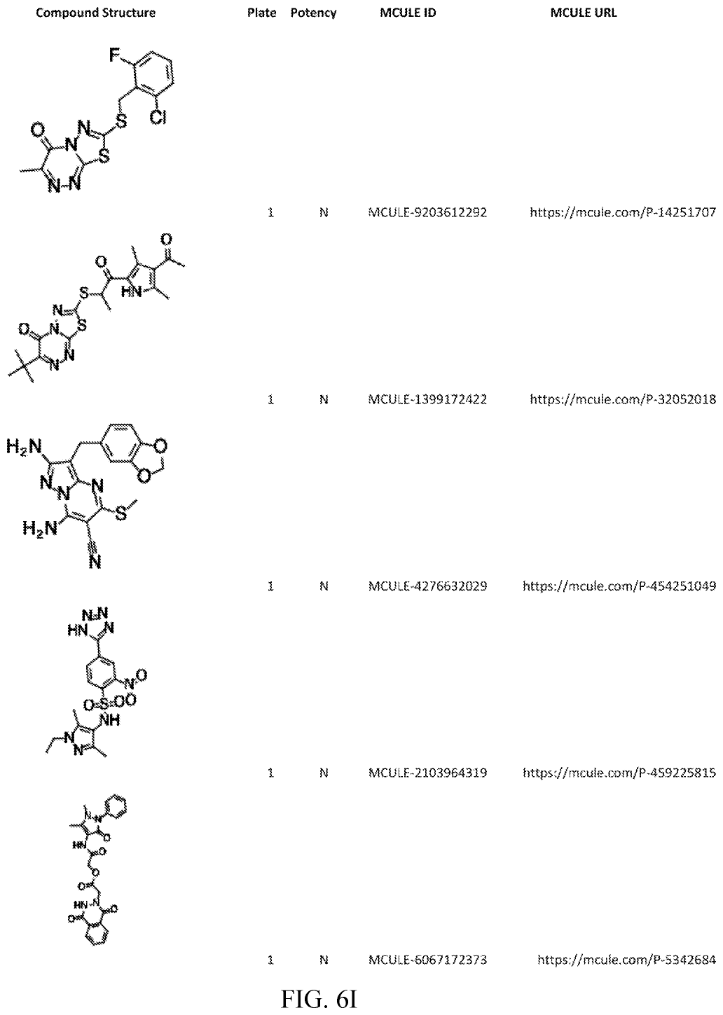

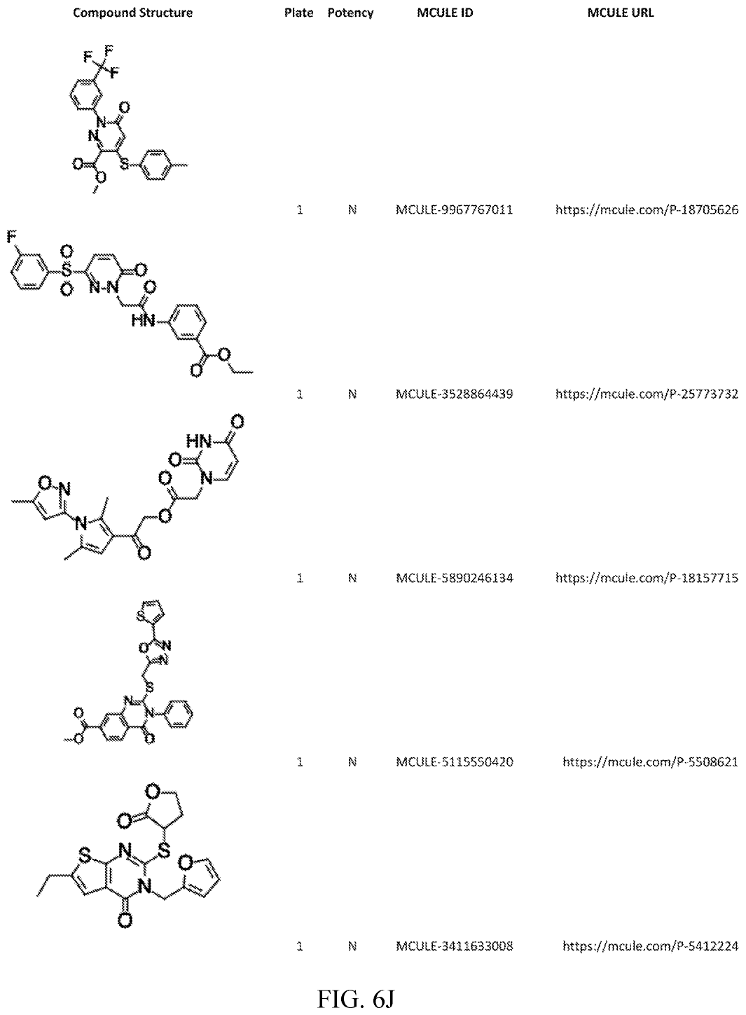

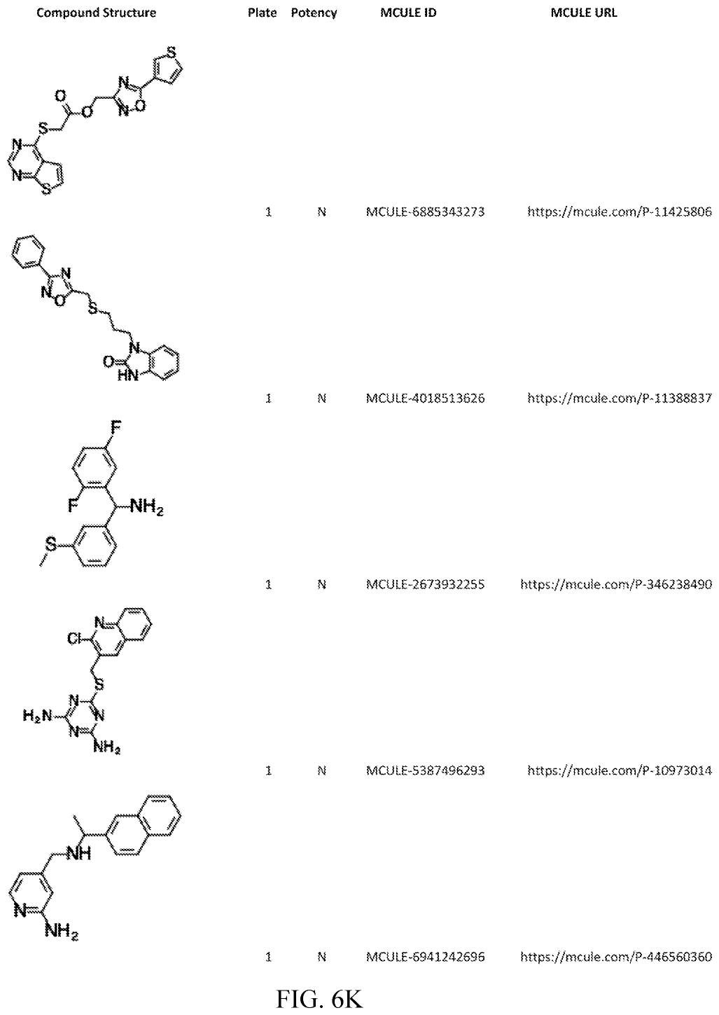

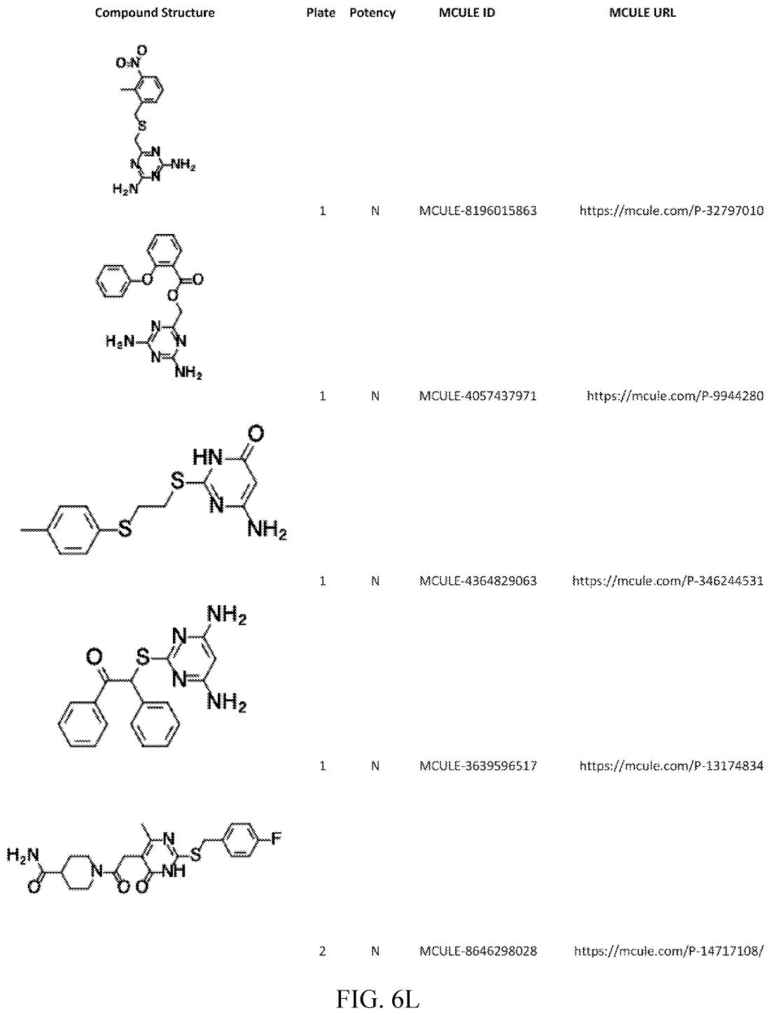

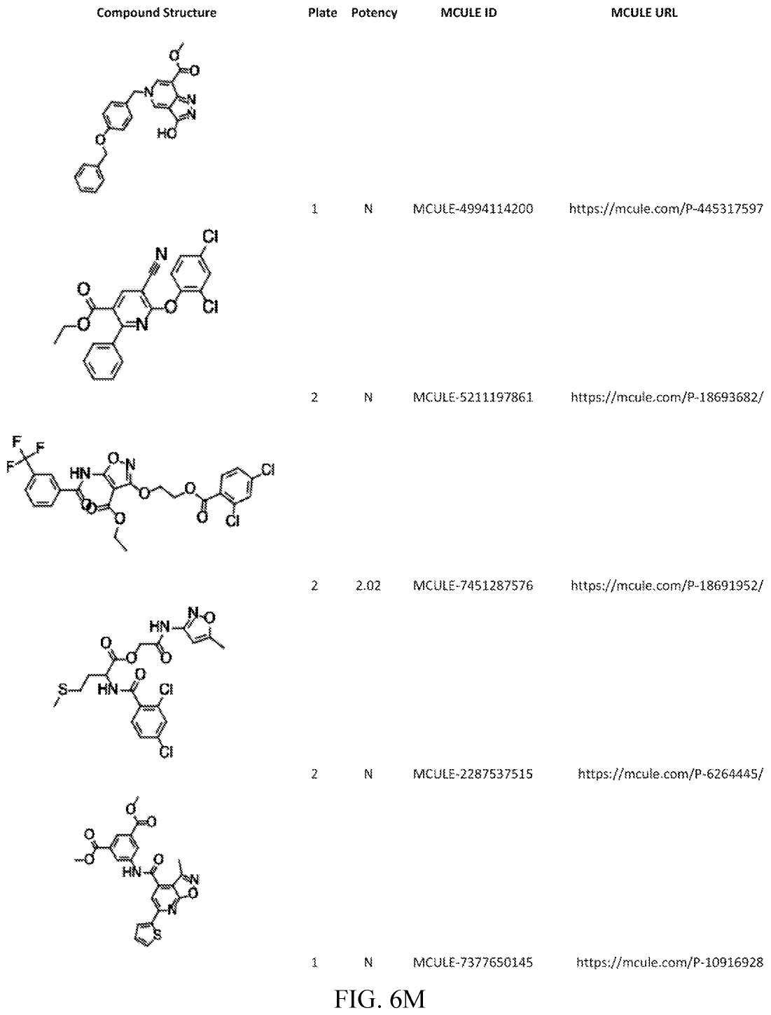

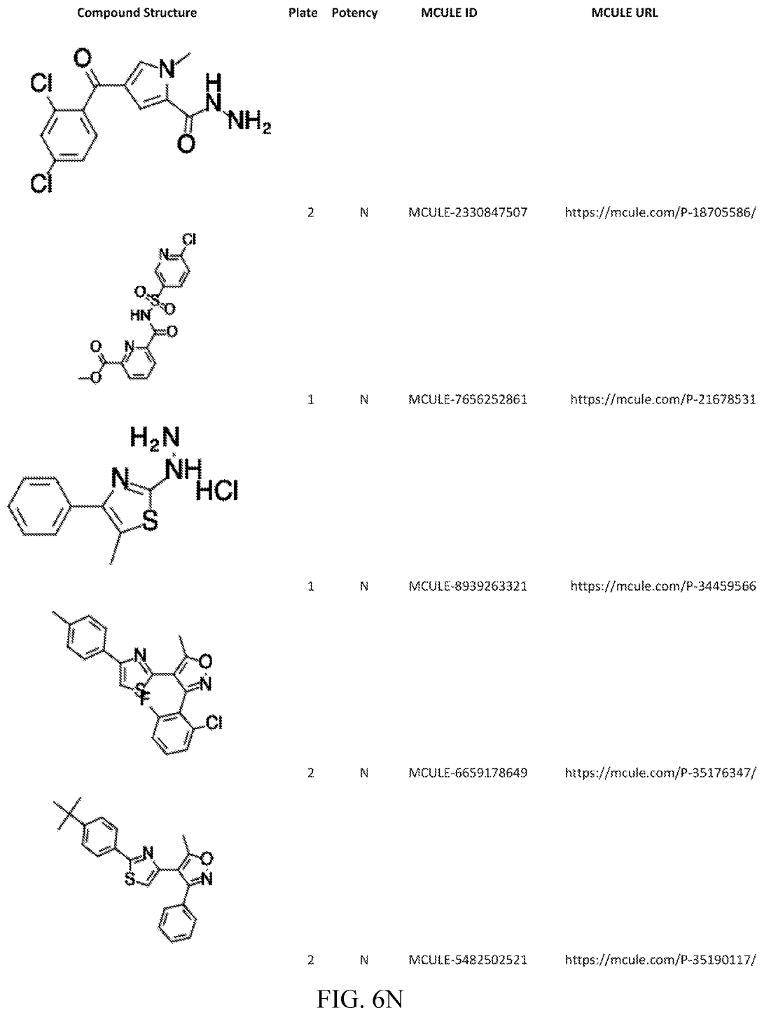

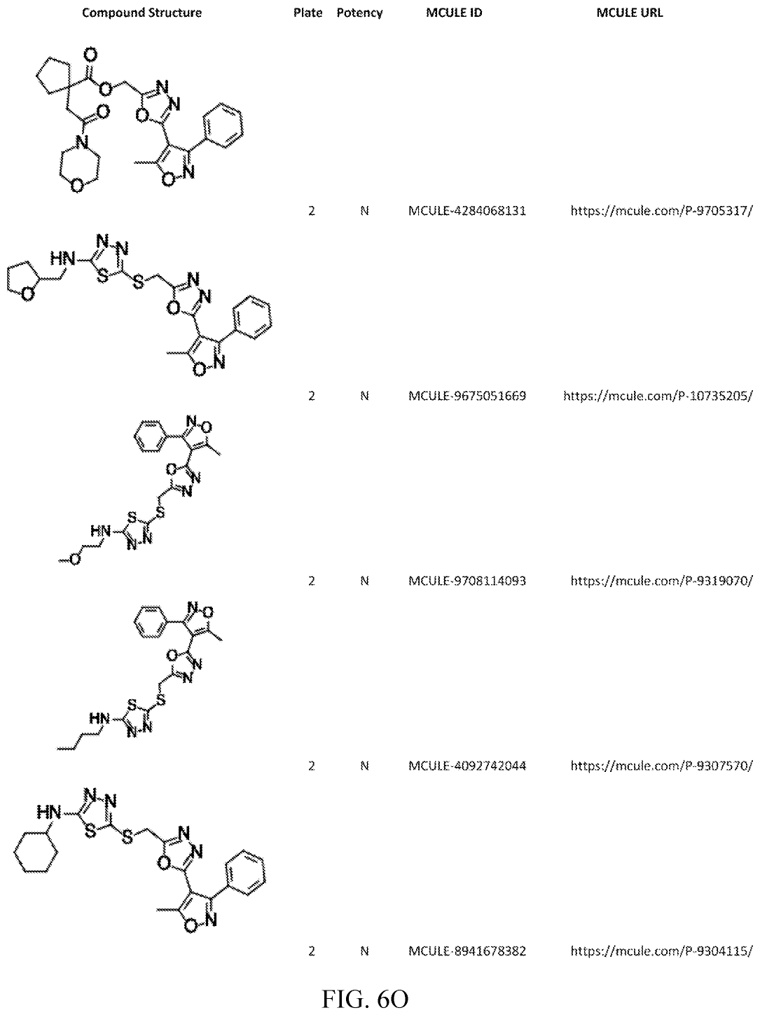

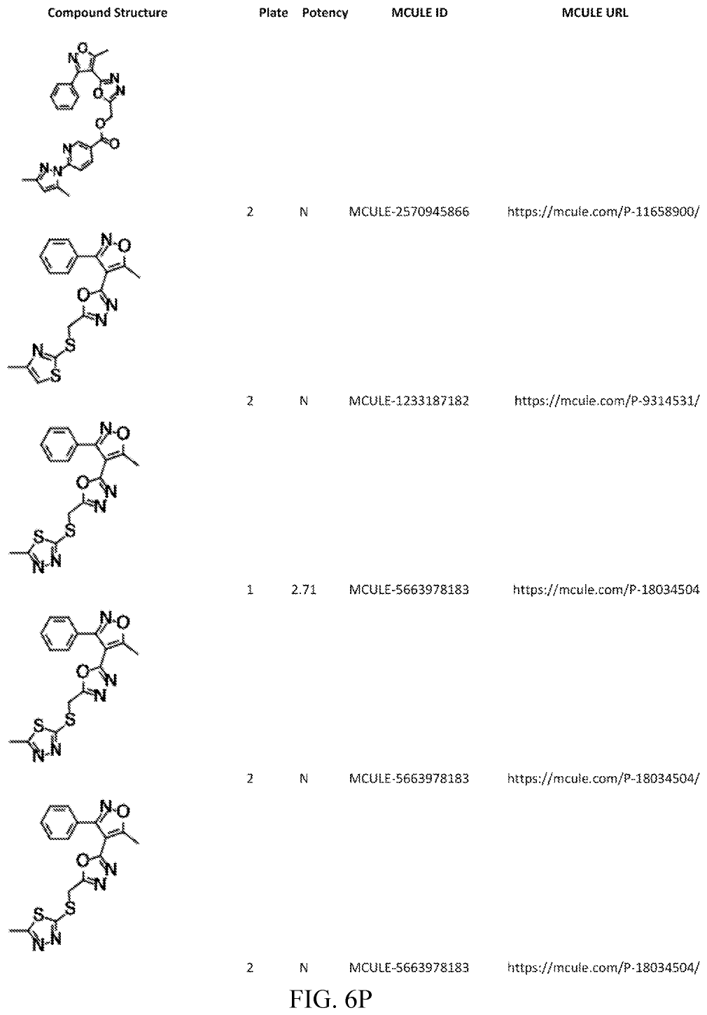

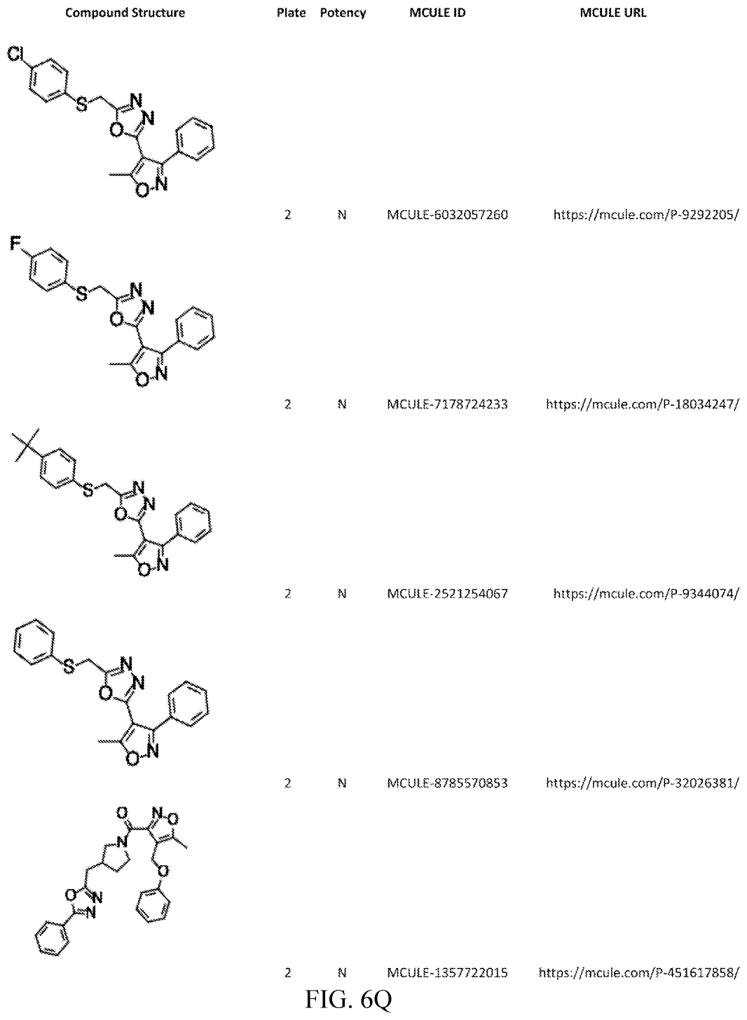

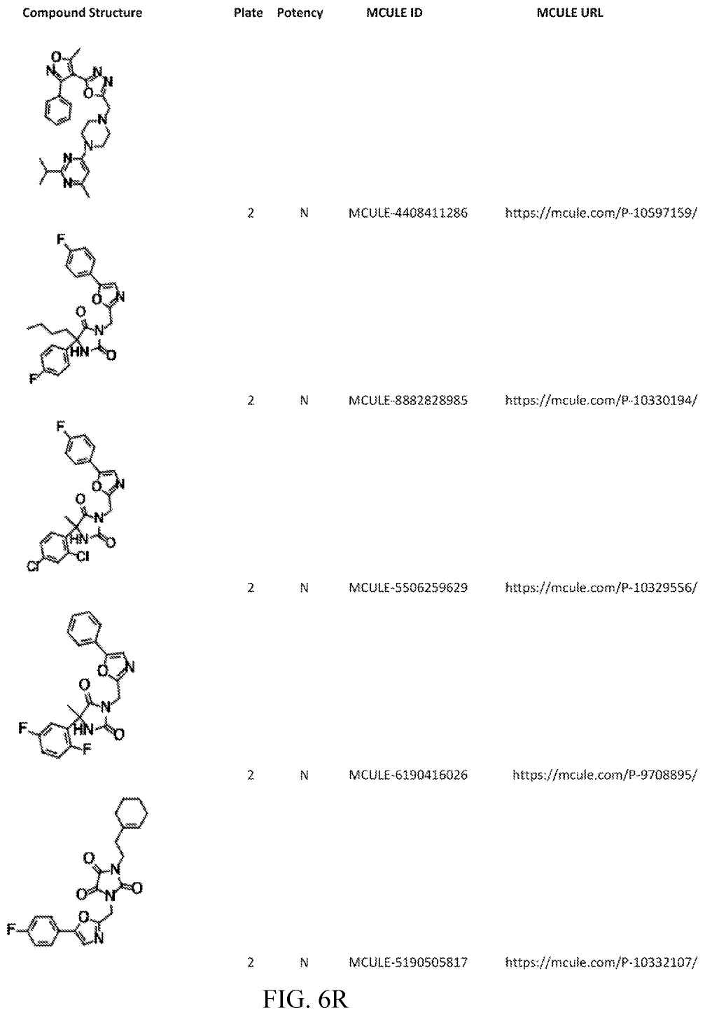

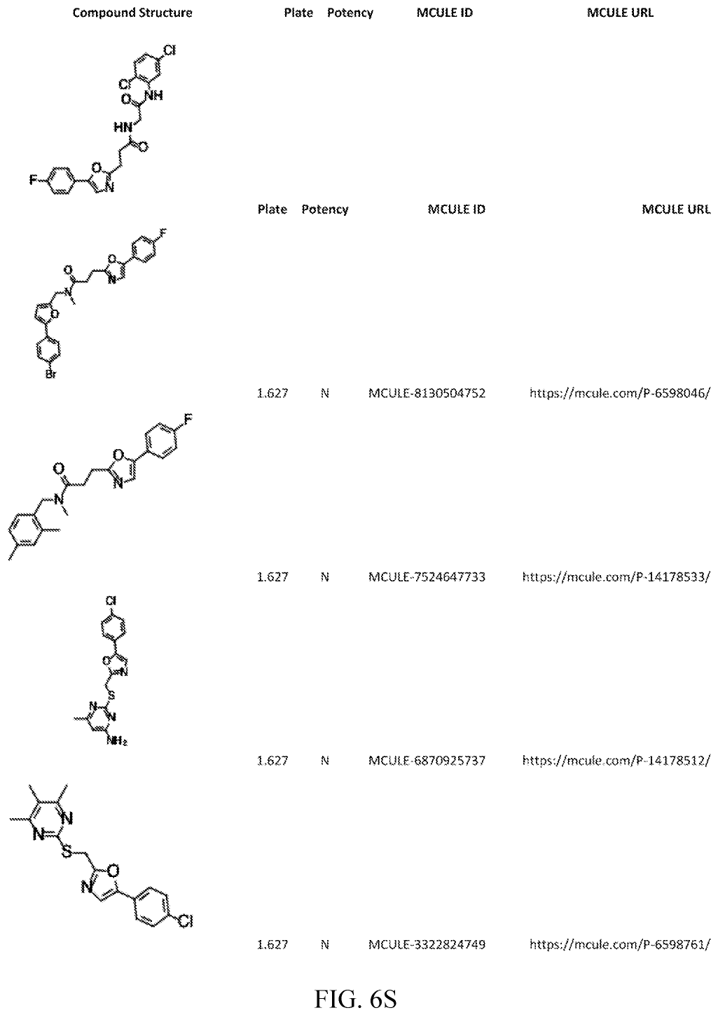

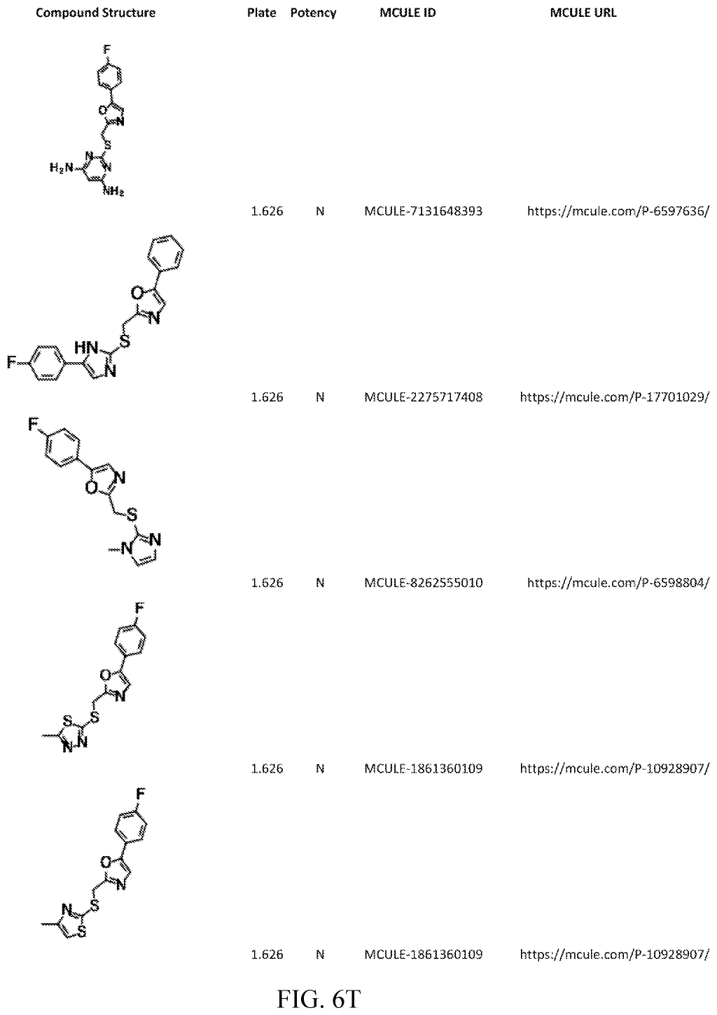

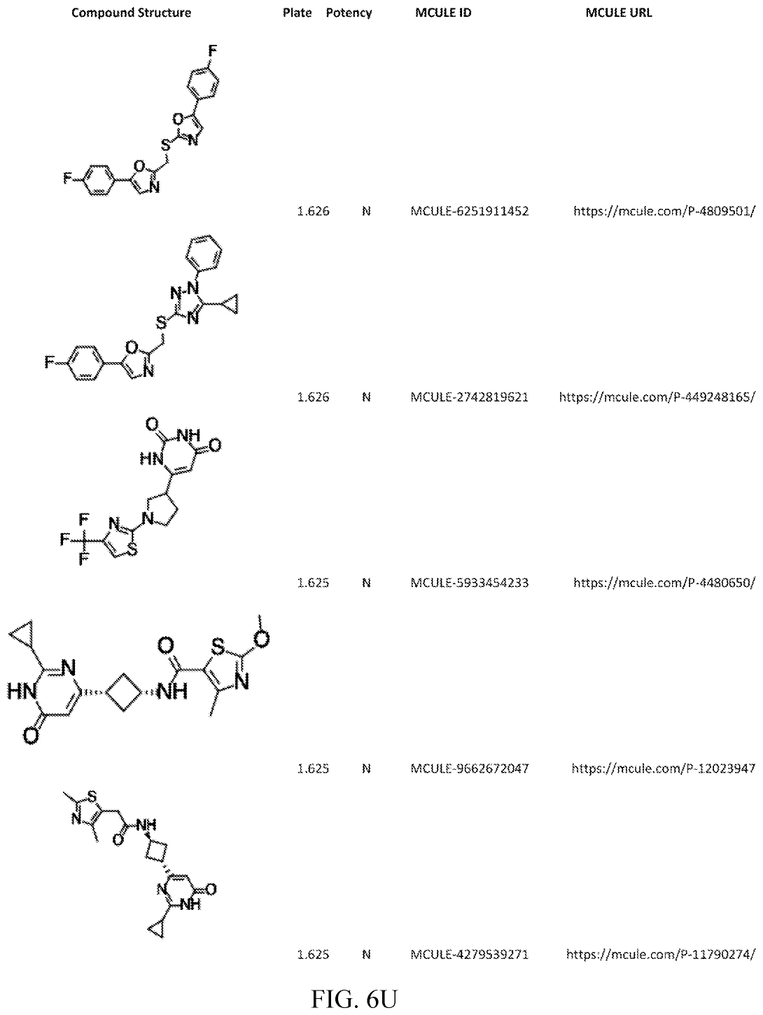

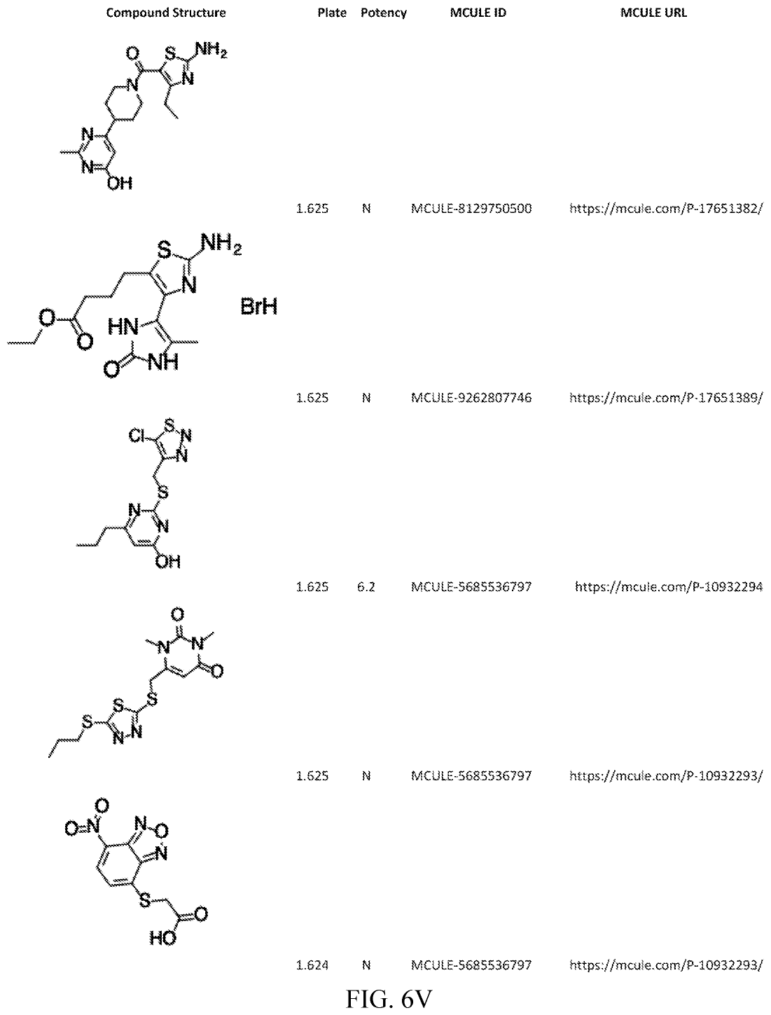

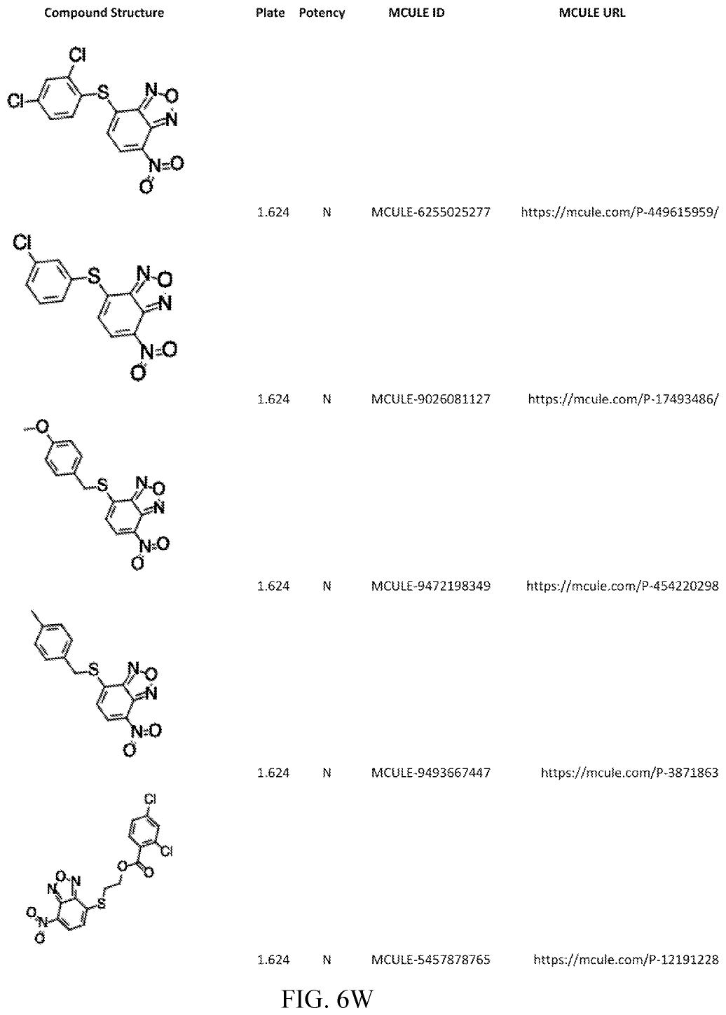

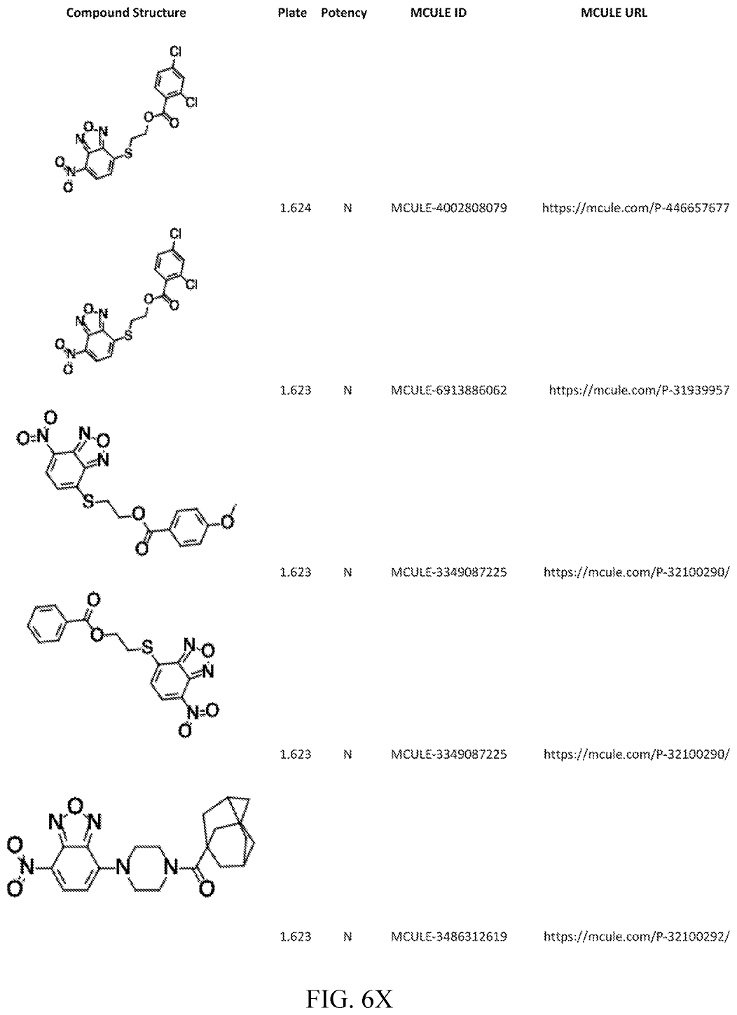

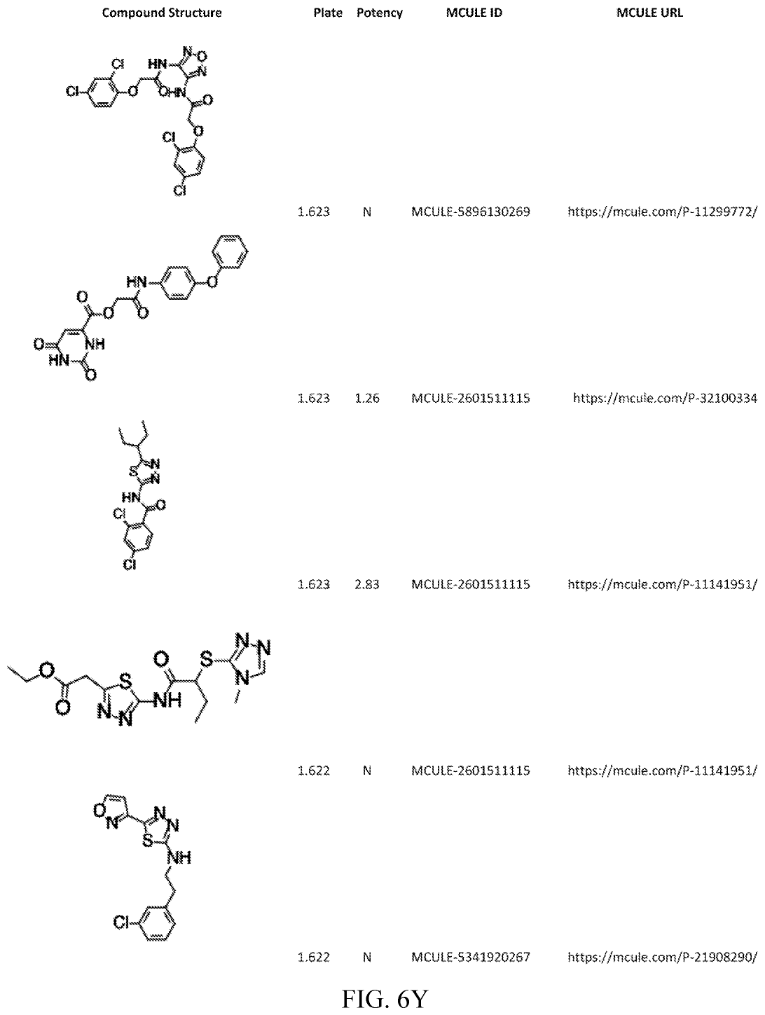

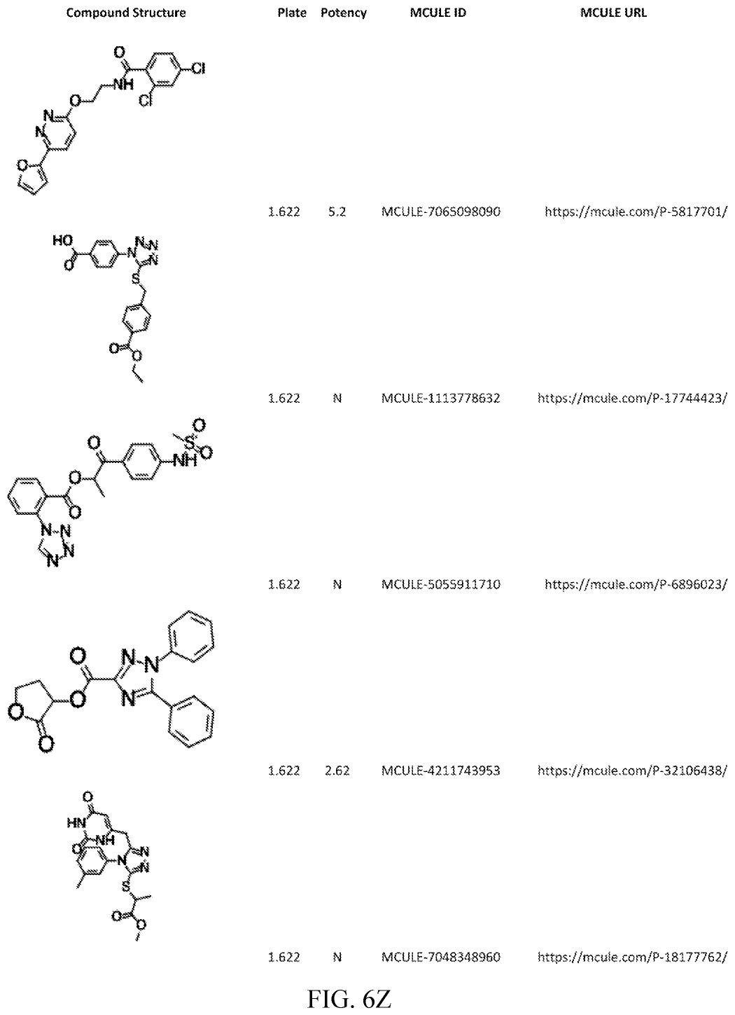

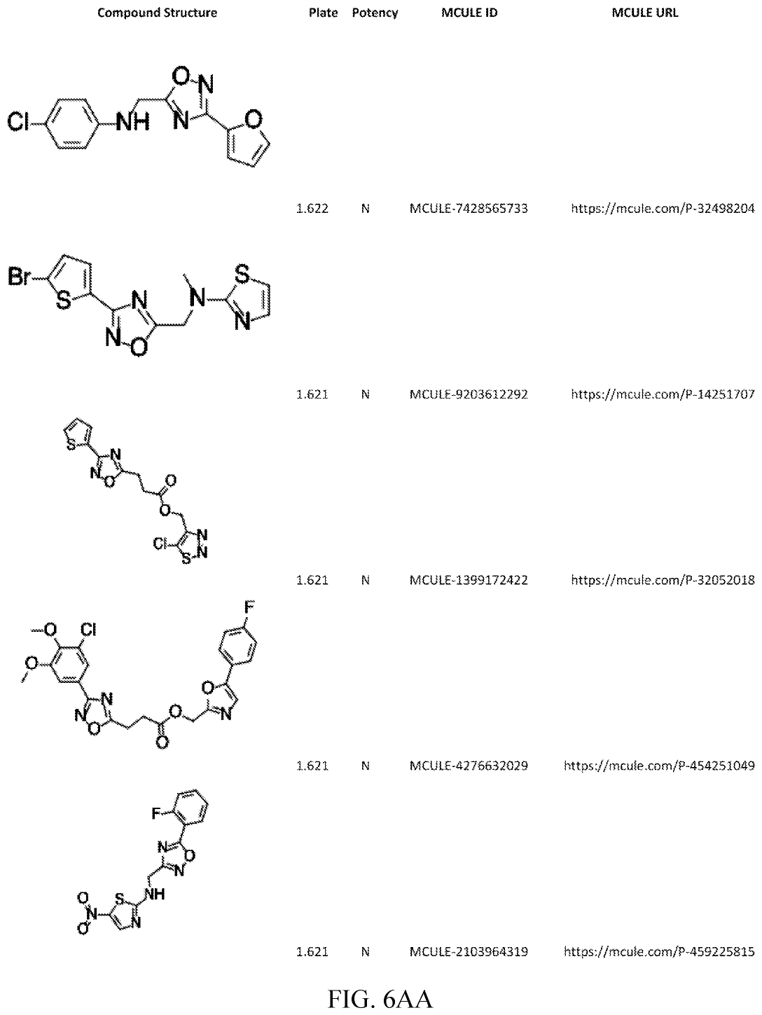

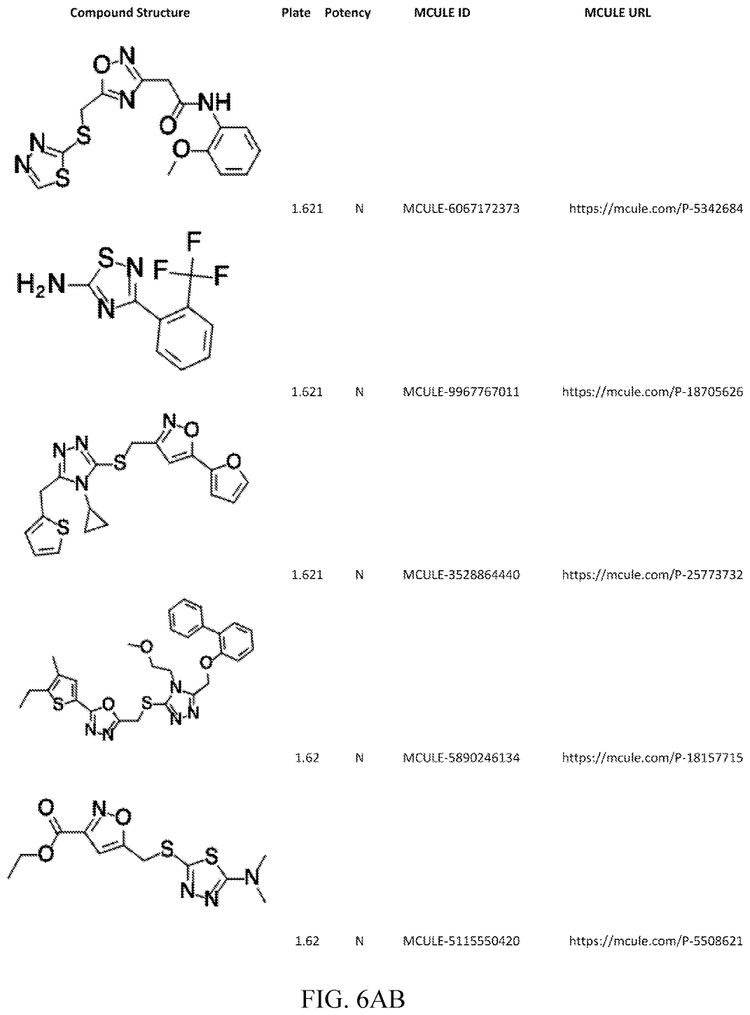

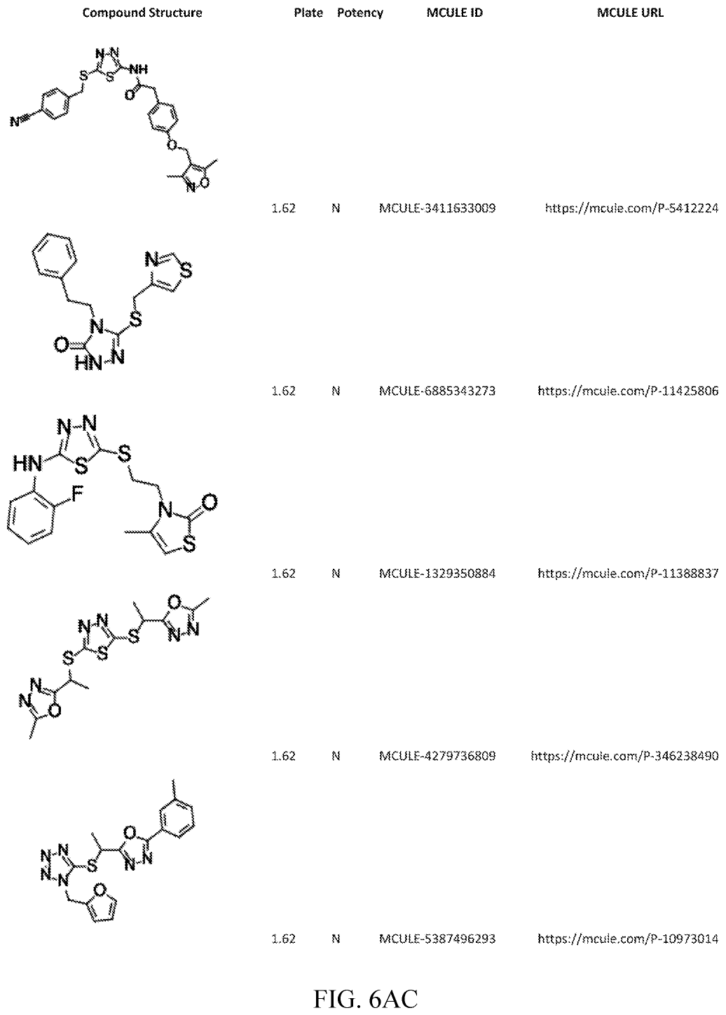

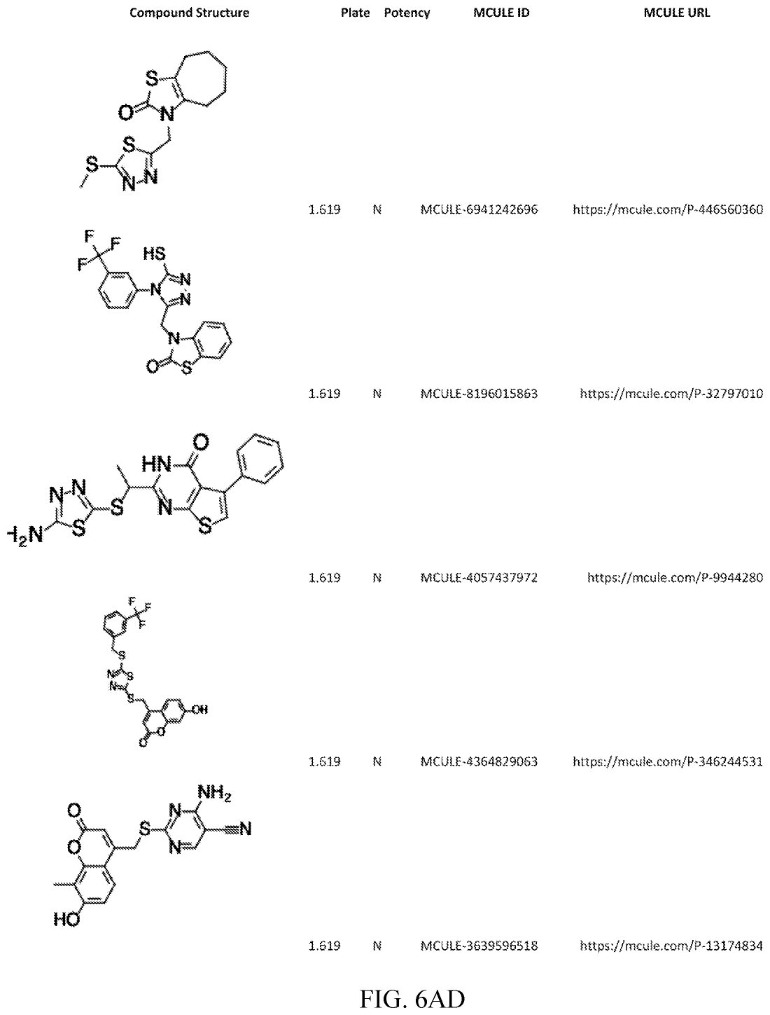

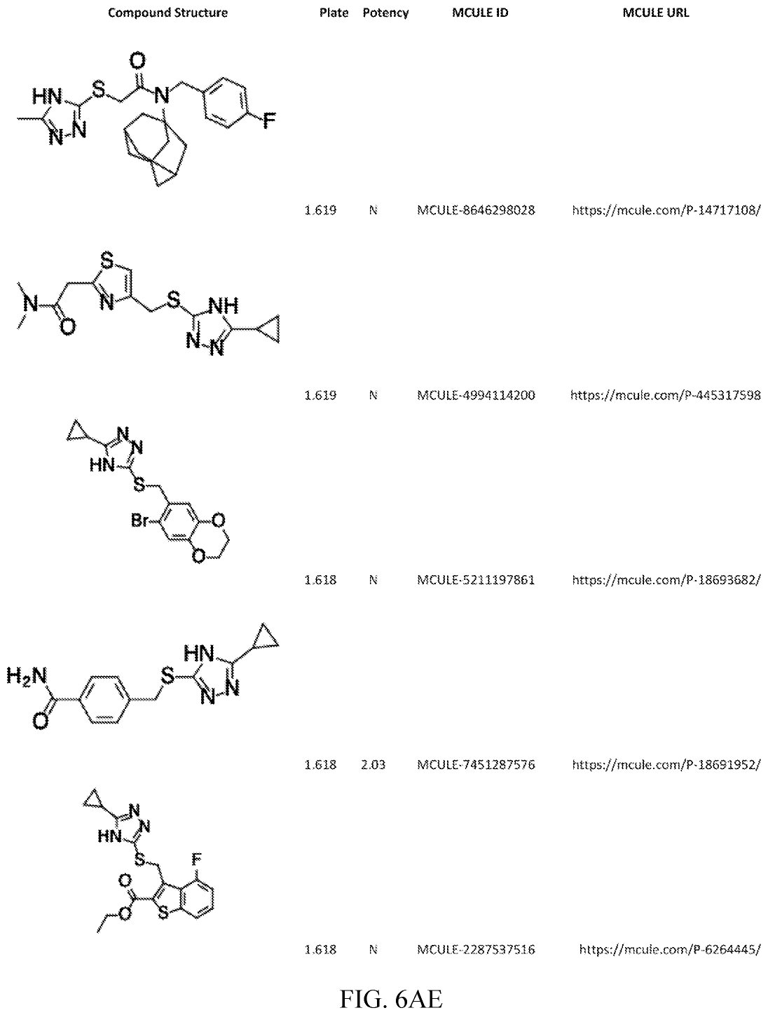

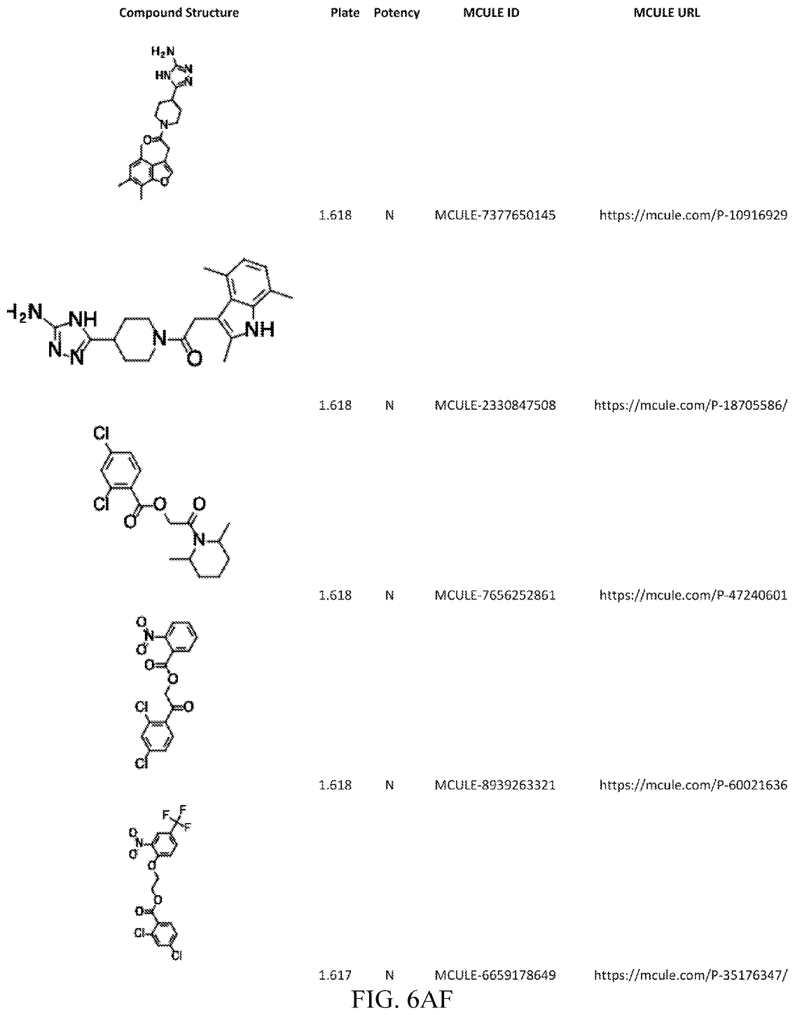

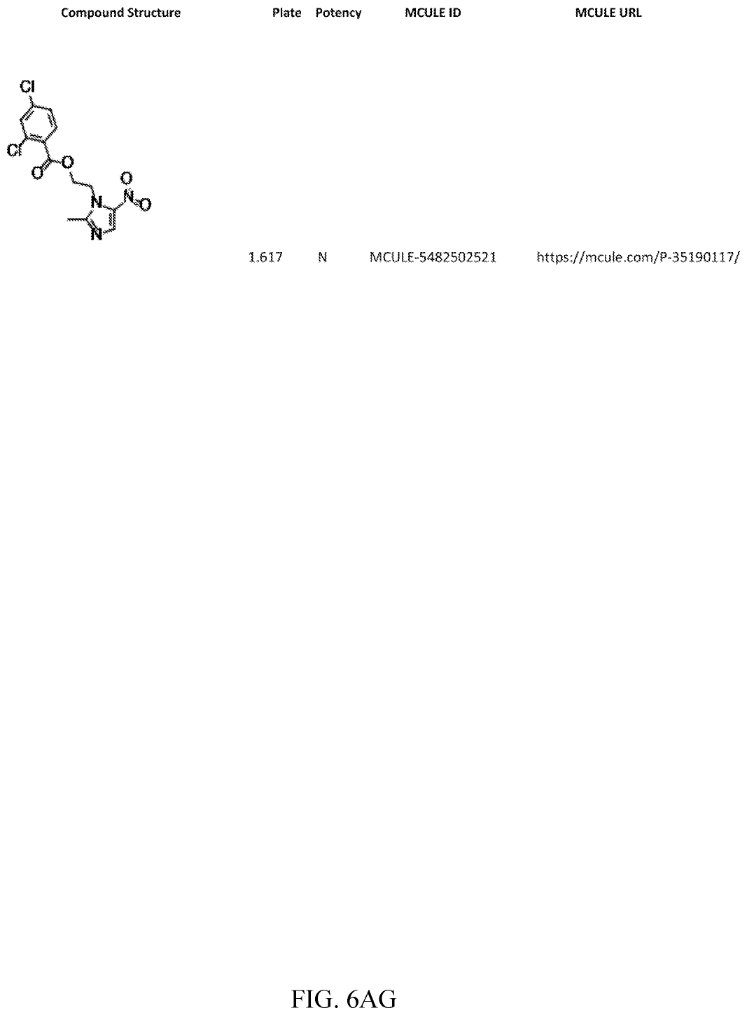

An additional number of compounds (FIGS. 6A-6AG) were screened and tested as described herein. A selected number of active compounds are as follows:

| | | Potency | Compound IDs |
|---|---|---|---|
| Compound Structures | Plate | (μM) | MCULE URL |
| 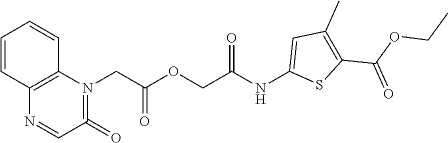 | 1 | 6.1 | Formula 4030 MCULE-5685536797 mcule.com/P-10932293 |
| 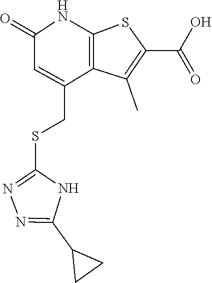 [SMILES: Cc1c(C(=O)O)sc2[nH]c(=O)cc(CSc3nnc(C4CC4)[nH]3)c12] | 1 | 1.25 | MCULE-2601511115 mcule.com/P-32100333 |
| 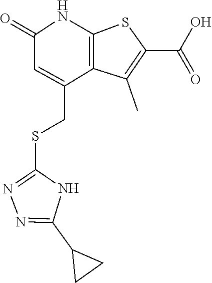 [SMILES: Cc1c(C(=O)O)sc2[nH]c(=O)cc(CSc3nnc(C4CC4)[nH]3)c12] | 2 | 2.82 | MCULE-2601511115 mcule.com/P-11141951/ |
| 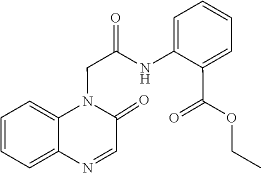 [SMILES: CCOC(=O)c1ccccc1NC(=O)Cn1c(=O)cnc2ccccc21] | 2 | 5.1 | Formula 2024 MCULE-7065098090 mcule.com/P-5817701/ |
| 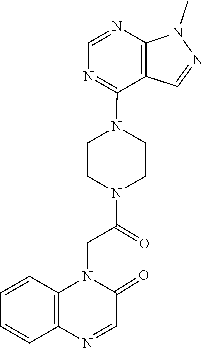 [SMILES: Cn1ncc2c(N3CCN(C(=O)Cn4c(=O)cnc5ccccc54)CC3)ncnc21] | 2 | 2.61 | Formula 2025 MCULE-4211743952 mcule.com/P-32106438/ |
| 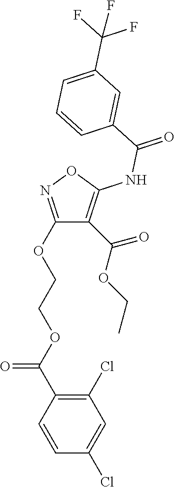 [SMILES: CCOC(=O)c1c(OCCOC(=O)c2ccc(Cl)cc2Cl)noc1NC(=O)c1cccc(C(F)(F)F)c1] | 2 | 2.02 | MCULE-745 1287576 mcule.com/P-18691952/ |
| 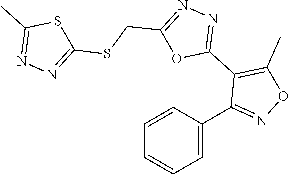 [SMILES: Cc1nnc(SCc2nnc(-c3c(-c4ccccc4)noc3C)o2)s1] | 1 | 2.71 | MCULE-5663978183 mcule.com/P-18034504 |
| 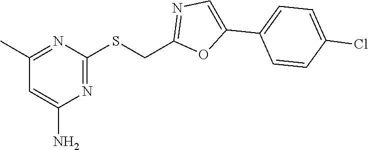 [SMILES: Cc1cc(N)nc(SCc2ncc(-c3ccc(Cl)cc3)o2)n1] | 2 | 2.42 | Formula 1024 MCULE-3601639289 mcule.com/P-32031735/ |
| 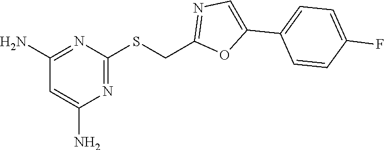 [SMILES: Nc1cc(N)nc(SCc2ncc(-c3ccc(F)cc3)o2)n1] | 1 | 4.82 | MCULE-6867206395 mcule.com/P-32058317 |
| 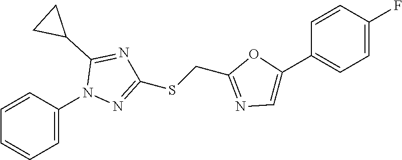 [SMILES: Fc1ccc(-c2cnc(CSc3nc(C4CC4)n(-c4ccccc4)n3)o2)cc1] | 2 | 3.57 | MCULE-2115384197 mcule.com/P-9955252/ |
| 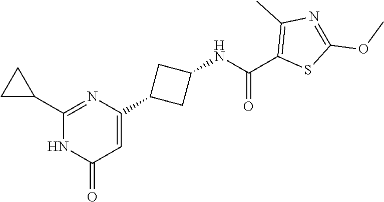 [SMILES: COc1nc(C)c(C(=O)N[C@H]2C[C@@H](c3cc(=O)[nH]c(C4CC4)n3)C2)s1] | 2 | 3.62 | MCULE-4390413901 mcule.com/P- 427423218/ |
| 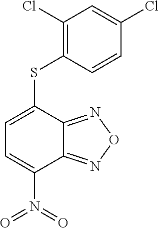 [SMILES: O=[N+]([O-])c1ccc(Sc2ccc(Cl)cc2Cl)c2nonc12] | 2 | 1.89 | Formula 4024 MCULE-9477245751 mcule.com/P-35227859/ |
| 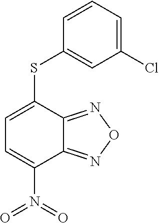 [SMILES: O=[N+]([O-])c1ccc(Sc2cccc(Cl)c2)c2nonc12] | 2 | 2.19 | Formula 4025 MCULE-9868926212 mcule.com/P-35227811/ |
| 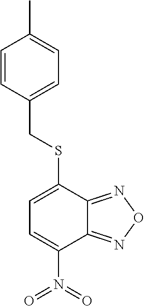 [SMILES: Cc1ccc(CSc2ccc([N+](=O)[O-])c3nonc23)cc1] | 2 | 3.28 | Formula 4026 MCULE-9444991753 mcule.com/P-35229090/ |
| 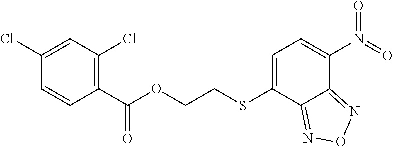 [SMILES: O=C(OCCSc1ccc([N+](=O)[O-])c2nonc12)c1ccc(Cl)cc1Cl] | 1 | 0.4 | Formula 4027 MCULE-2879769428 mcule.com/P-35226327 |
| 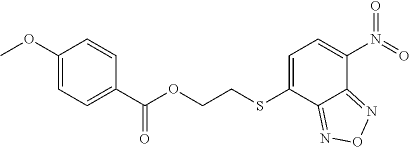 [SMILES: COc1ccc(C(=O)OCCSc2ccc([N+](=O)[O-])c3nonc23)cc1] | 2 | 3.19 | Formula 4028 MCULE-1664189451 mcule.com/P-35226503/ |
| 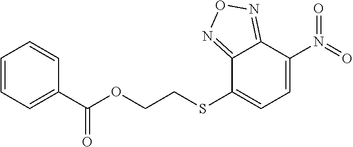 [SMILES: O=C(OCCSc1ccc([N+](=O)[O-])c2nonc12)c1ccccc1] | 2 | 1.61 | Formula 4029 MCULE-2580483274 mcule.com/P-35227071/ |

Synthesis of Compounds

ANAT inhibitors described herein can be synthesized by methods known in the art. For example, the benzoxadiazole inhibitor series compounds can be synthesized, without limitation, as follows:

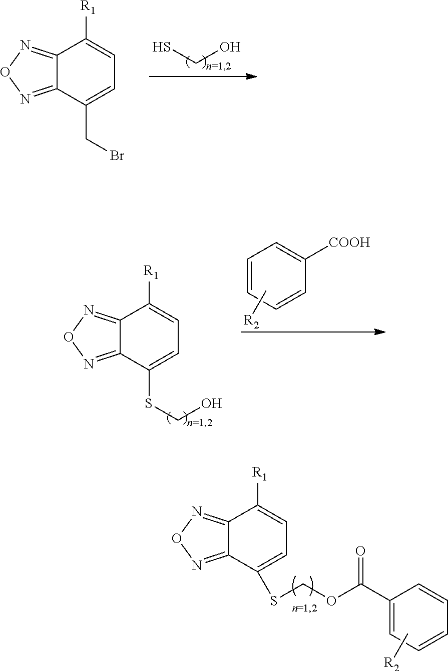

where R₁ can be selected from, without limitation, nitro, carboxyl, and the like, and R₂ can be selected from, without limitation, methyl, methoxy, chloro, dichloro, and the like.

A number of patent and non-patent publications are cited herein in order to describe the state of the art to which this disclosure pertains. The entire disclosure of each of these publications is incorporated by reference herein.

While certain embodiments of the present disclosure have been described and/or exemplified above, various other embodiments will be apparent to those skilled in the art from the foregoing disclosure. The present disclosure is, therefore, not limited to the particular embodiments described and/or exemplified, but is capable of considerable variation and modification without departure from the scope and spirit of the appended claims.

Further details describing the state of the art to which this disclosure pertains are provided in the references below:

### REFERENCES

Davies, K., 1993. The cause of Canavan's disease. Nature Genetics, 365, pp. 1-1.
Guo, F. et al., 2015. Ablating N-acetylaspartate prevents leukodystrophy in a Canavan disease model. Annals of Neurology, 77(5), pp. 884-888.
Janson, C. G. et al., 2006. Natural history of Canavan disease revealed by proton magnetic resonance spectroscopy (1H-MRS) and diffusion-weighted MRI. Neuropediatrics, 37(4), pp. 209-221.
Kaul, R. et al., 1993. Cloning of the human aspartoacylase cDNA and a common missense mutation in Canavan disease. Nature Genetics, 5(2), pp. 118-123.
Leone, P. et al., 2012. Long-term follow-up after gene therapy for canavan disease. Science translational medicine, 4(165), pp. 165ra163-165ra163.
Maier, H. et al., 2015. N-Acetylaspartate Synthase Deficiency Corrects the Myelin Phenotype in a Canavan Disease Mouse Model But Does Not Affect Survival Time. The Journal of neuroscience: the official journal of the Society for Neuroscience, 35(43), pp. 14501-14516.
Matalon, R. et al., 1988. Aspartoacylase deficiency and N-acetylaspartic aciduria in patients with Canavan disease. American journal of medical genetics, 29(2), pp. 463-471.
Tahay, G. et al., 2012. Determinants of the enzymatic activity and the subcellular localization of aspartate N-acetyltransferase. Biochemical Journal, 441(1), pp. 105-112.
Thangavelu, B. et al., 2017. Design and optimization of aspartate N-acetyltransferase inhibitors for the potential treatment of Canavan disease. Bioorganic & Medicinal Chemistry, 25(3), pp. 870-885.
Wallach, I., Dzamba, M. & Heifets, A., 2015. AtomNet: A Deep Convolutional Neural Network for Bioactivity Prediction in Structure-based Drug Discovery. arXiv.org, cs.LG.
Wang, Q. et al., 2016. Purification and characterization of aspartate N-acetyltransferase: A critical enzyme in brain metabolism. Protein Expression and Purification, 119, pp. 11-18.

1. A method of treating or preventing a disease alleviated by inhibiting ANAT in a subject in need thereof, comprising administering to the subject a therapeutically effective amount of a compound of Formula IV, Formula V, Formula I, Formula II, Formula III-a, Formula III-b, Formula VI, or Formula VII: 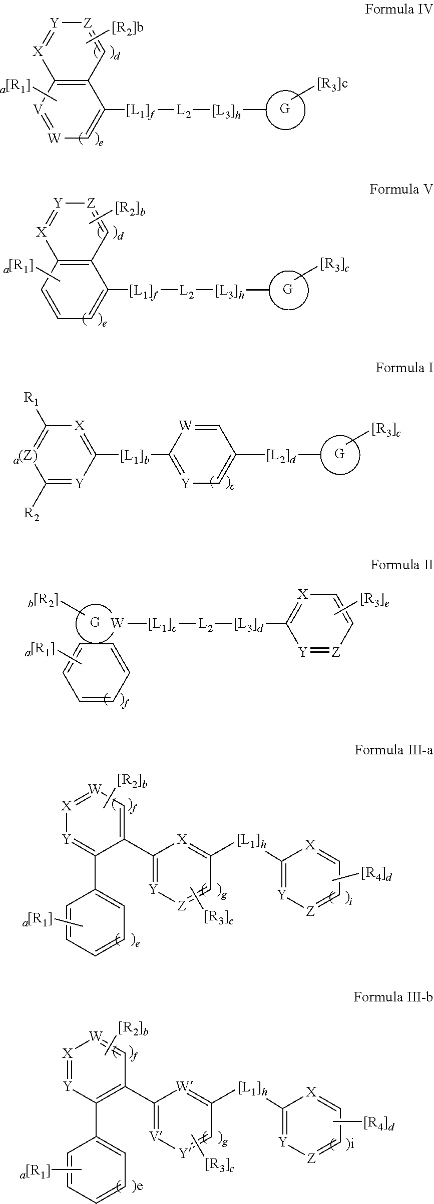 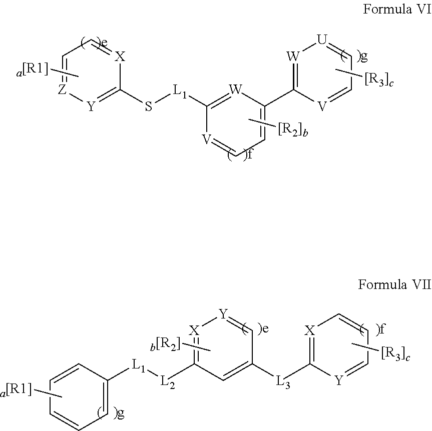
or a pharmaceutically acceptable analog, derivative, salt, solvate, hydrate, cocrystal, or prodrug thereof, wherein in Formulas I to VII:
U, V, W, V′, W′, Y′, X, Y, and Z are independently selected at each occurrence from C, CH, CH₂, N, NH, NR^{a}, O, S
G is independently at each occurrence a mono- or polycyclic optionally substituted cycloalkyl, mono- or polycyclic optionally substituted heterocycloalkyl, mono- or polycyclic optionally substituted aryl, mono- or polycyclic optionally substituted arylalkyl, mono- or polycyclic optionally substituted heteroaryl, and mono- or polycyclic optionally substituted heteroarylalkyl;
R₁, R₂, and R₃ are independently selected at each occurrence from hydrogen, optionally substituted alkylheteroaryl, optionally substituted alkyl, optionally substituted heteroalkyl, optionally substituted alkenyl, optionally substituted alkynyl, optionally substituted cycloalkyl, optionally substituted heterocycloalkyl, optionally substituted fused heterocycloalkyl, optionally substituted aryl, optionally substituted arylalkyl, optionally substituted heteroaryl, optionally substituted alkylheteroaryl, optionally substituted heteroarylalkyl, optionally substituted alkoxy, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂, wherein R₁, R₂, and R₃ can optionally be joined to form a carbo- or heterocycle;
L₁, L₂, and L₃ are linkers comprising independently at each occurrence one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a}C(O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl;
wherein any optional substituent is independently selected at each occurrence from optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂;
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and
a, b, c, d, e, f, g, h, and i are independently at each occurrence an integer selected from 0, 1, 2, 3, 4, 5, and 6.

2. The method of claim 1, wherein G is independently at each occurrence selected from benzene, pyridine, pyrimidine, pyrazine, pyridazine, and triazine.

3. The method of claim 1, wherein G is independently at each occurrence selected from furan, thiophene, pyrrole, thiazole, isothiazole, 1,2,3-thiadiazole, imidazole, oxazole, isoxazole, 1,2,3-oxadiazole, 1,3,4-oxadiazole, 1,2,4-oxadiazole, 1,2,3-triazole, 1,2,4-triazole, and pyrazole.

4. The method of claim 1, wherein G is independently at each occurrence selected from naphthalene, quinoline, isoquinoline, cinnoline, quinoxaline, phtalazine, pyridopyrazine, pteridine, pyridopyridazine, naphtyridine, carbazole, dibenzofuran, or quinazoline.

5. The method of claim 1, wherein G is independently at each occurrence selected from indole, benzoxazole, benzothiophene, benzimidazole, indazole, benzotriazole, pyrrolopyridine, triazolopyridine, purine, indolizine, pyrrolopyrimidine, pyrrolopyrazine, pyrrolopyrimine, pyrrolopyridazine, imidazopyridine, pyrazolopyridine, imidazopyridazine, or imidazopyrimidine.

6. The method of claim 1, wherein the compound has a formula selected from any one of Formulas 1 to 5:

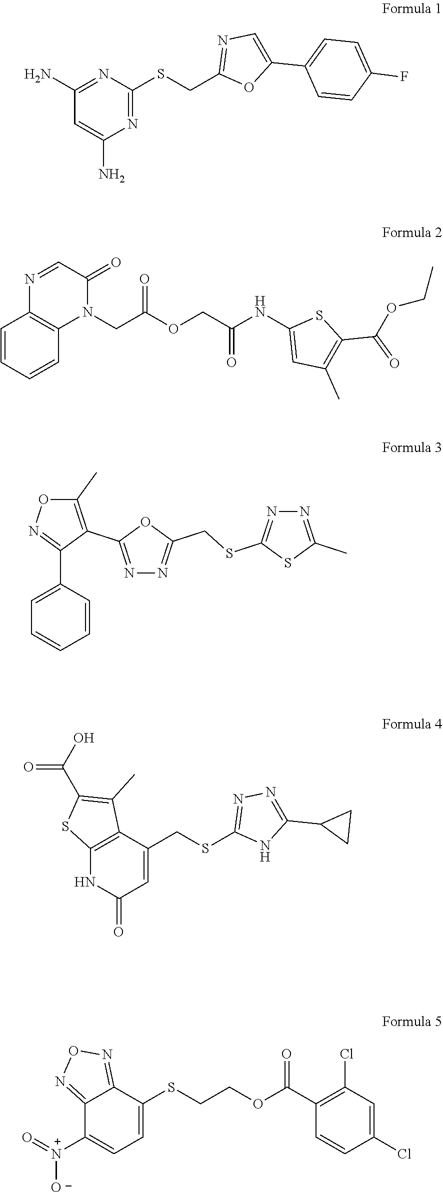 [SMILES: Nc1cc(N)nc(SCc2ncc(-c3ccc(F)cc3)o2)n1 ; CCOC(=O)c1sc(NC(=O)COC(=O)Cn2c(=O)cnc3ccccc32)cc1C ; Cc1nnc(SCc2nnc(-c3c(-c4ccccc4)noc3C)o2)s1 ; Cc1c(C(=O)O)sc2[nH]c(=O)cc(CSc3nnc(C4CC4)[nH]3)c12 ; O=C(OCCSc1ccc([N+](=O)[O-])c2nonc12)c1ccc(Cl)cc1Cl]

7. The method of claim 1, wherein the compound has a formula selected from any one of Formulas 1001 to 1024:

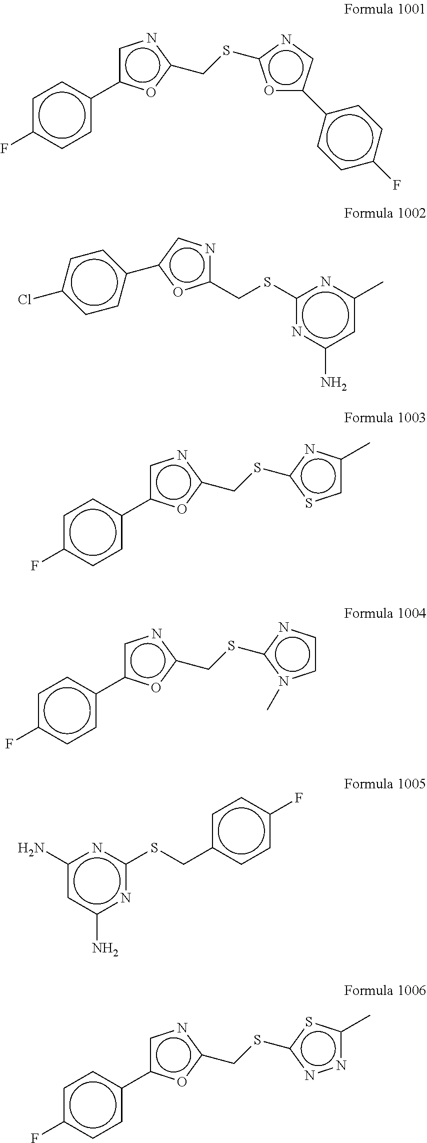

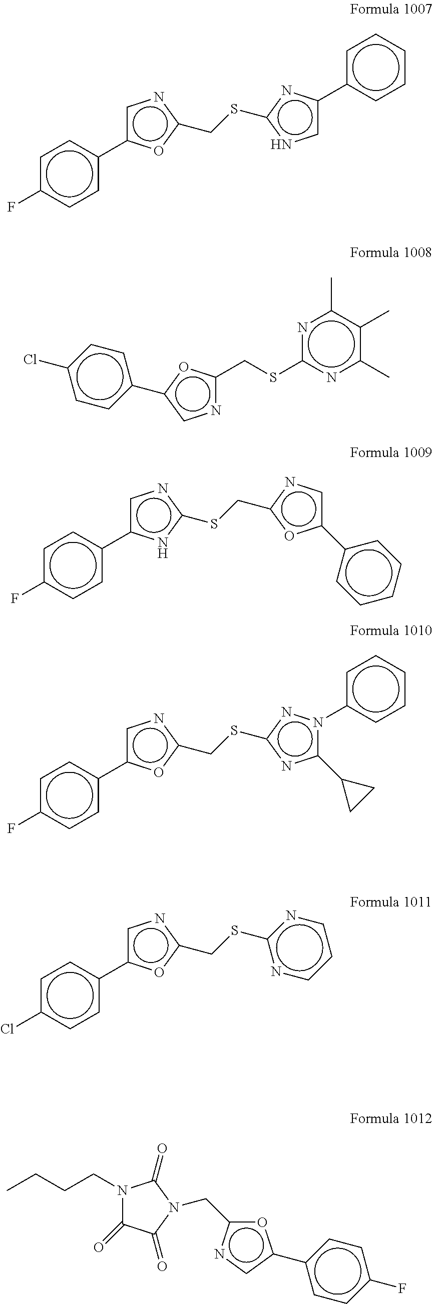

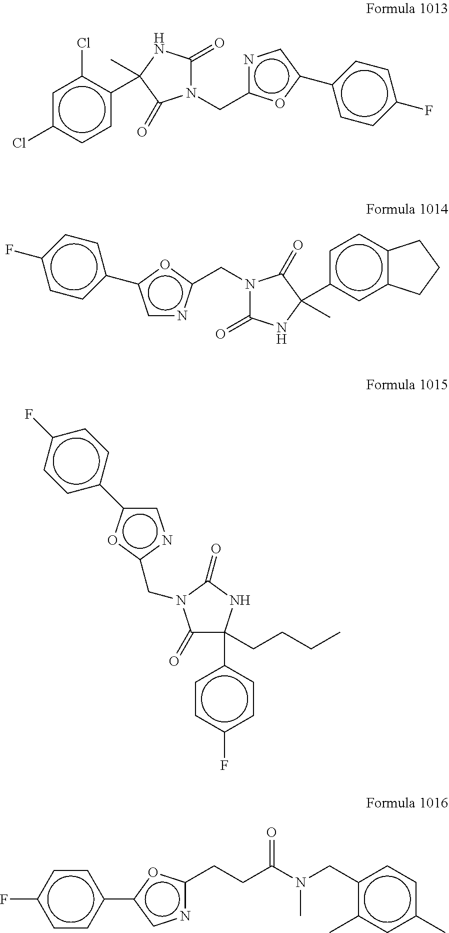

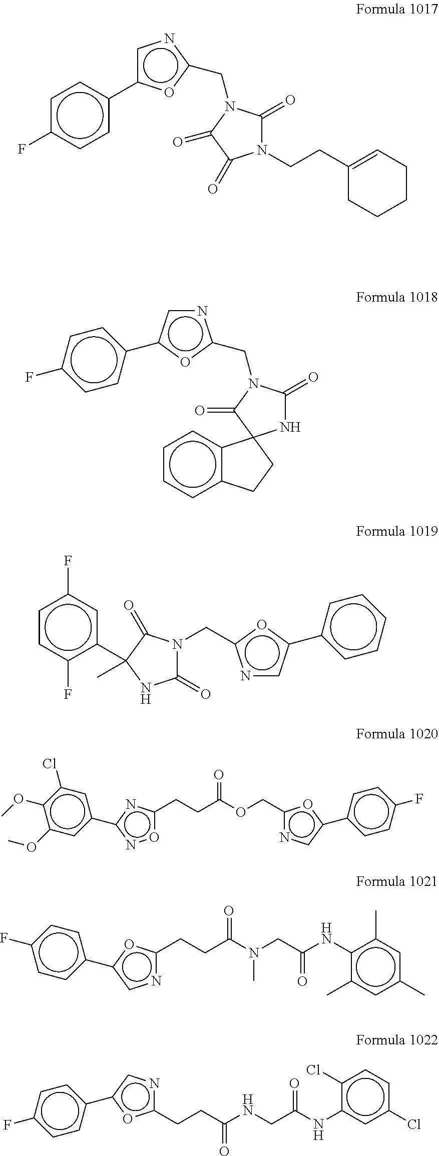

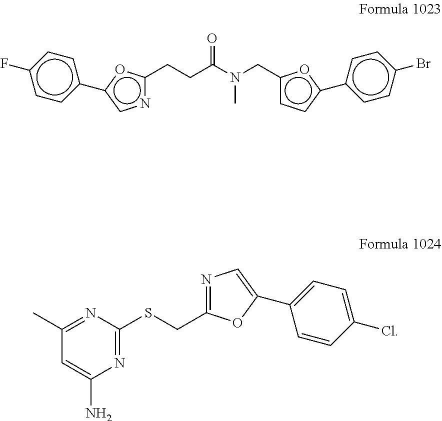

8. The method of claim 1, wherein the compound has a formula selected from any one of Formulas 2001 to 2025:

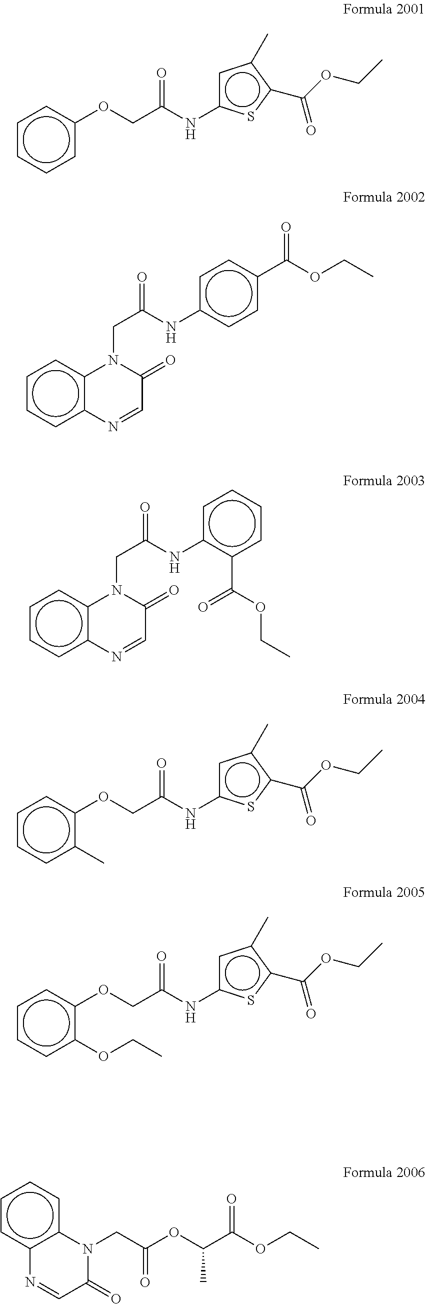

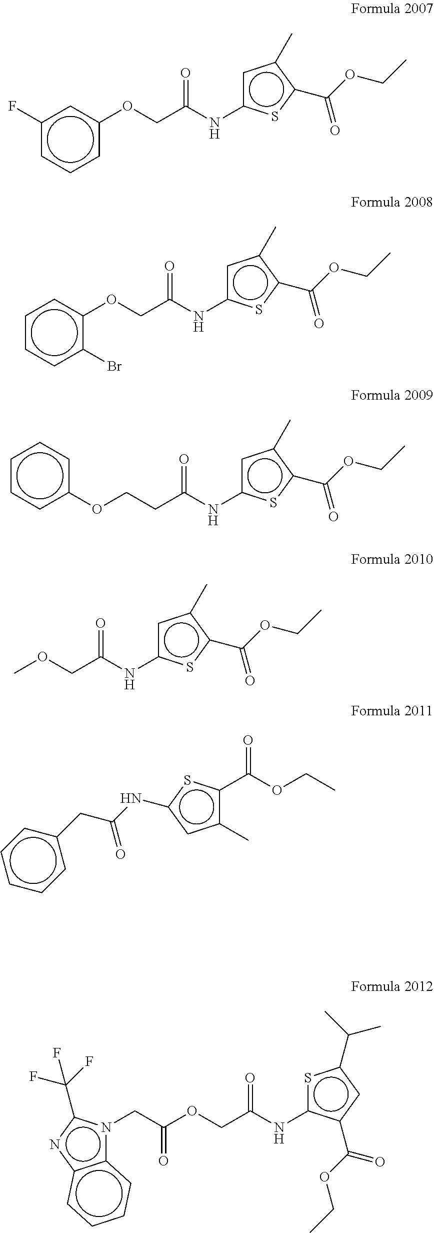

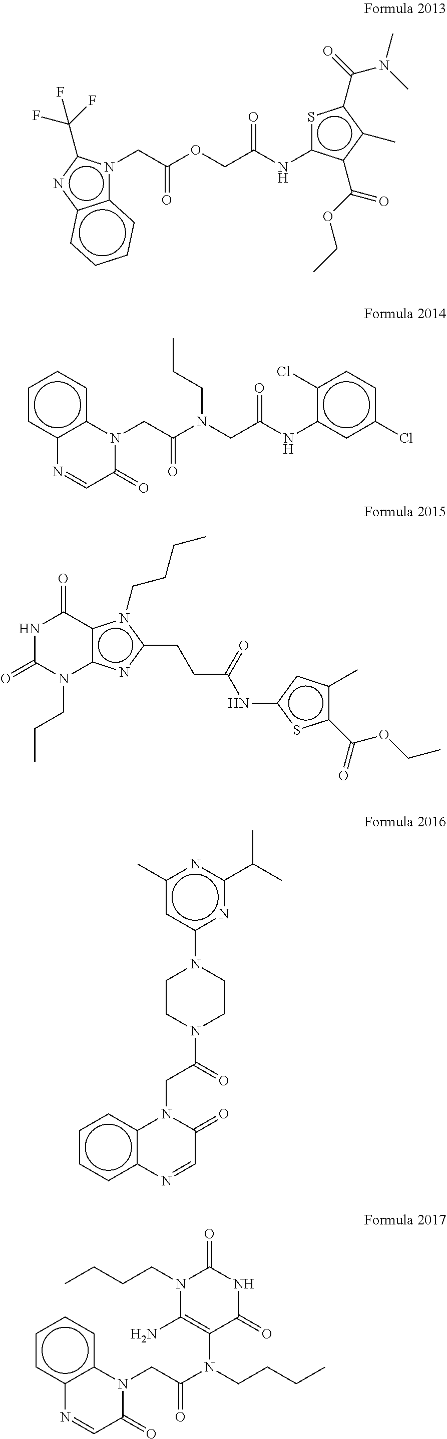

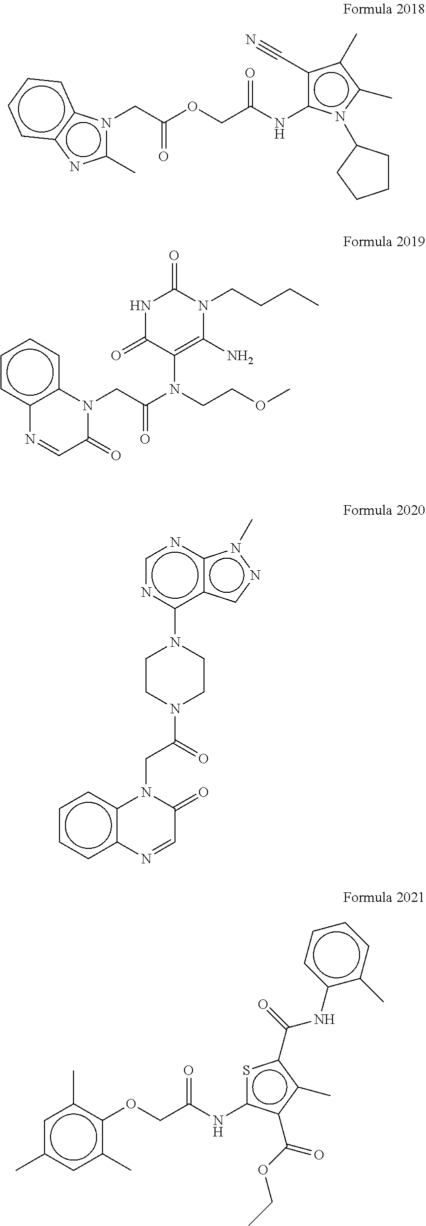

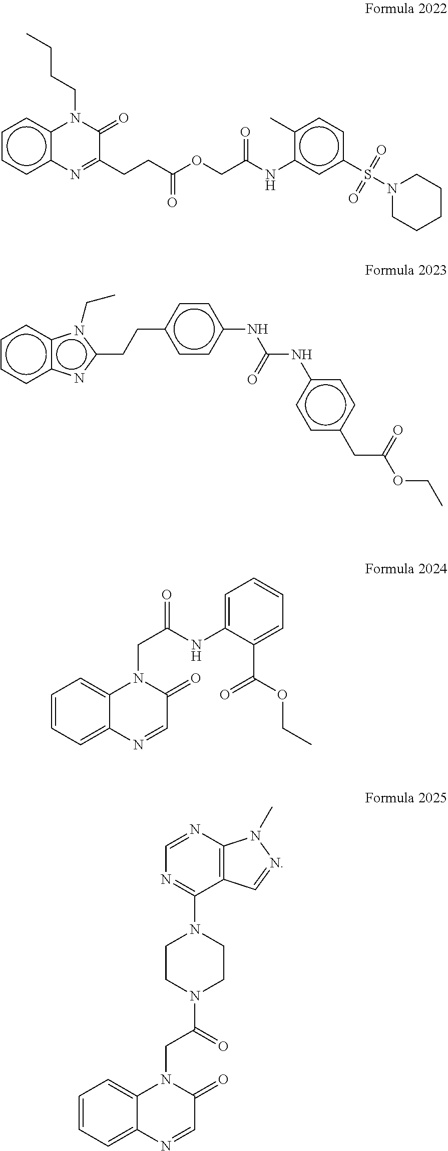

9. The method of claim 1, wherein the compound has a formula selected from any one of Formulas 3001 to 3023:

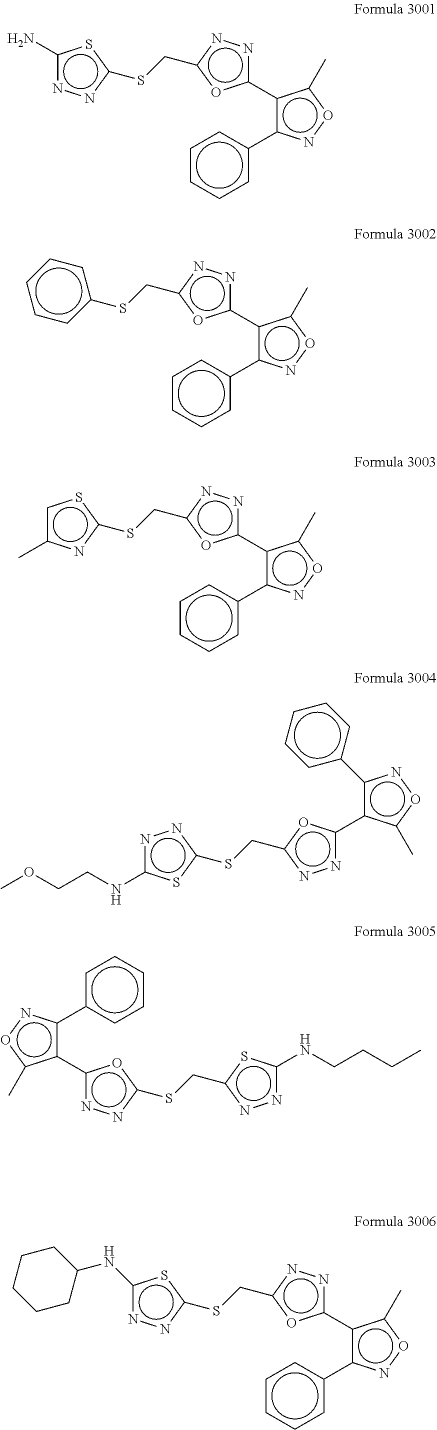

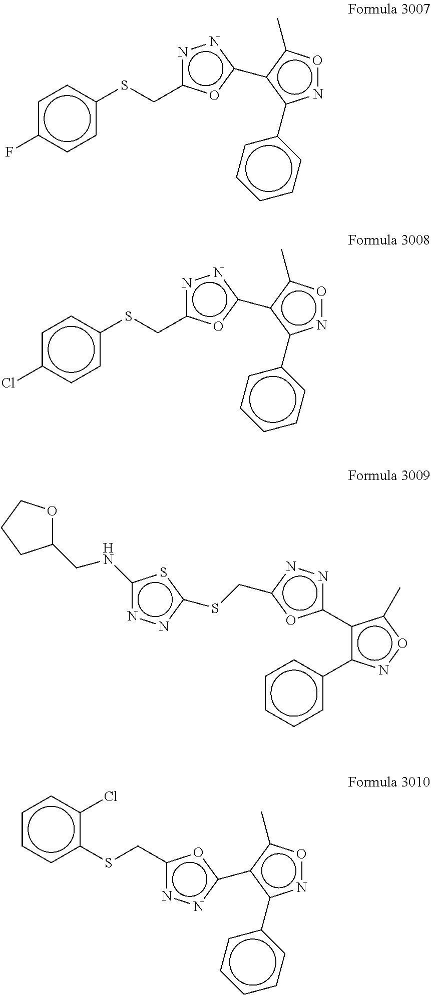

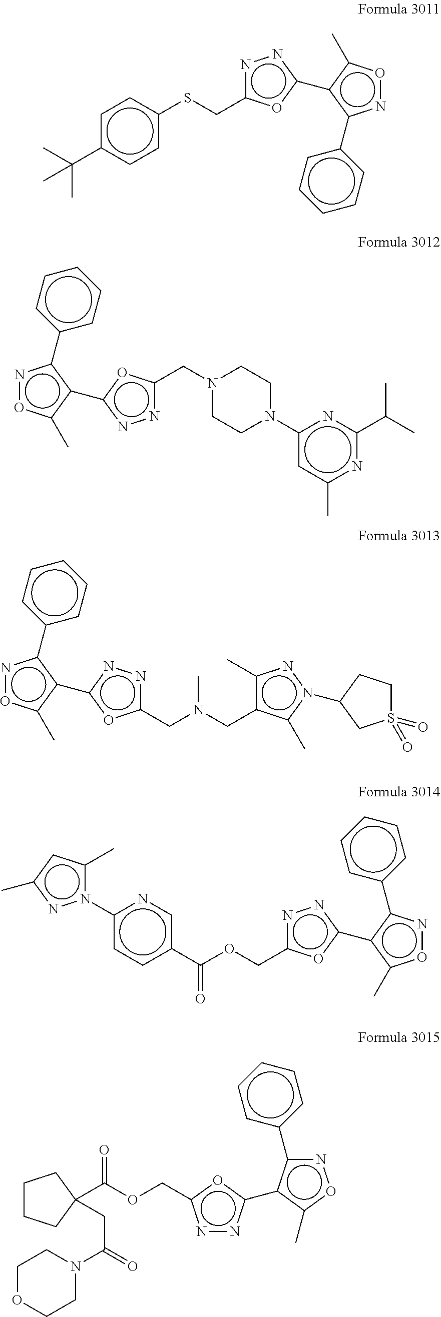

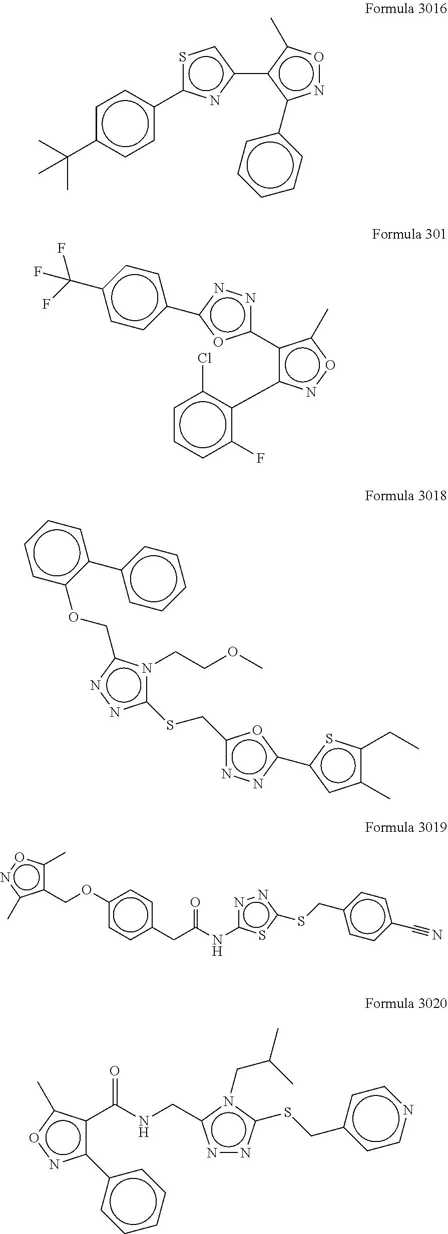

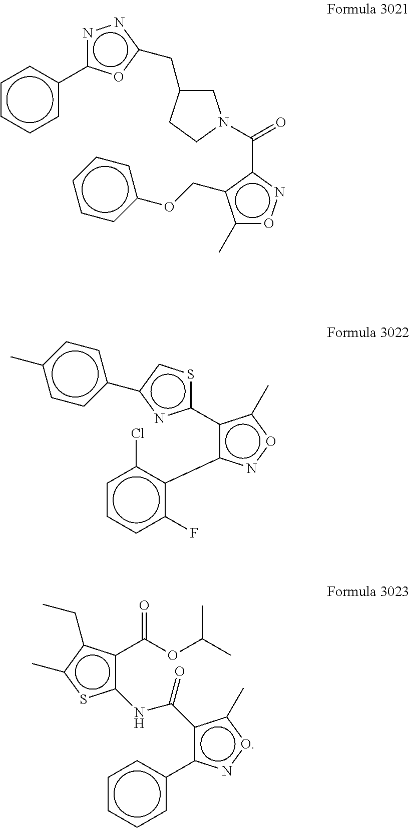

10. The method of claim 1, wherein the compound has a formula selected from any one of Formulas 4001 to 4030:

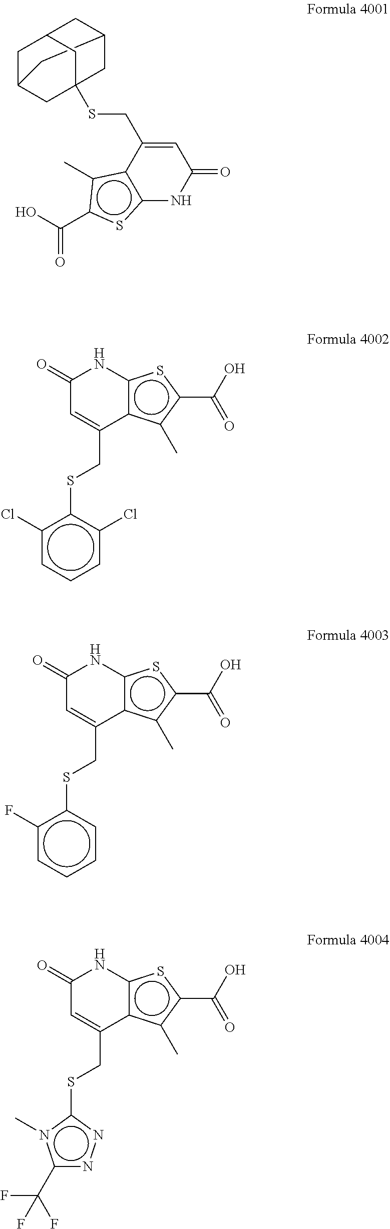

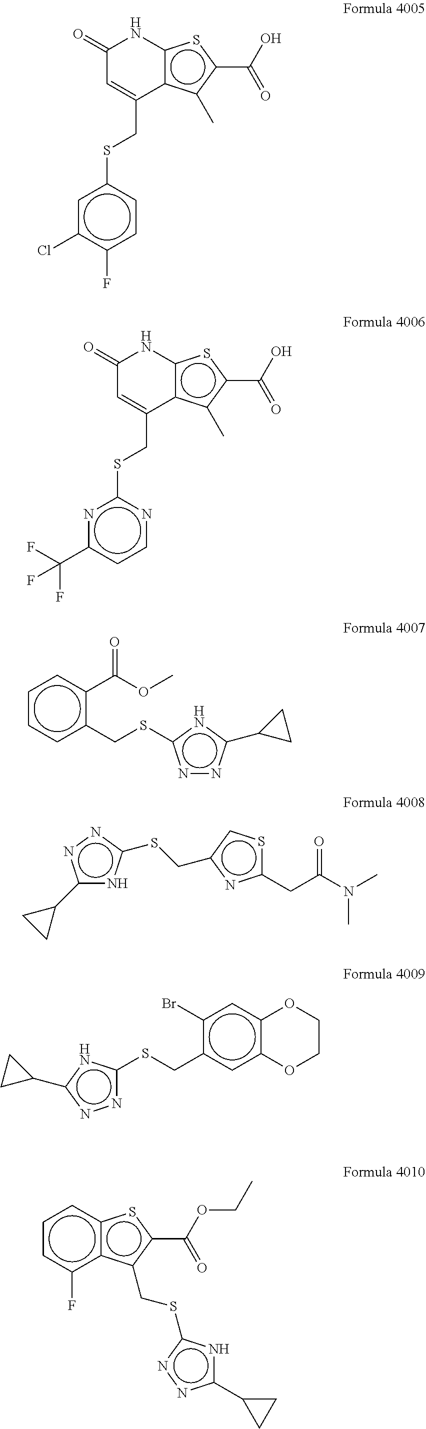

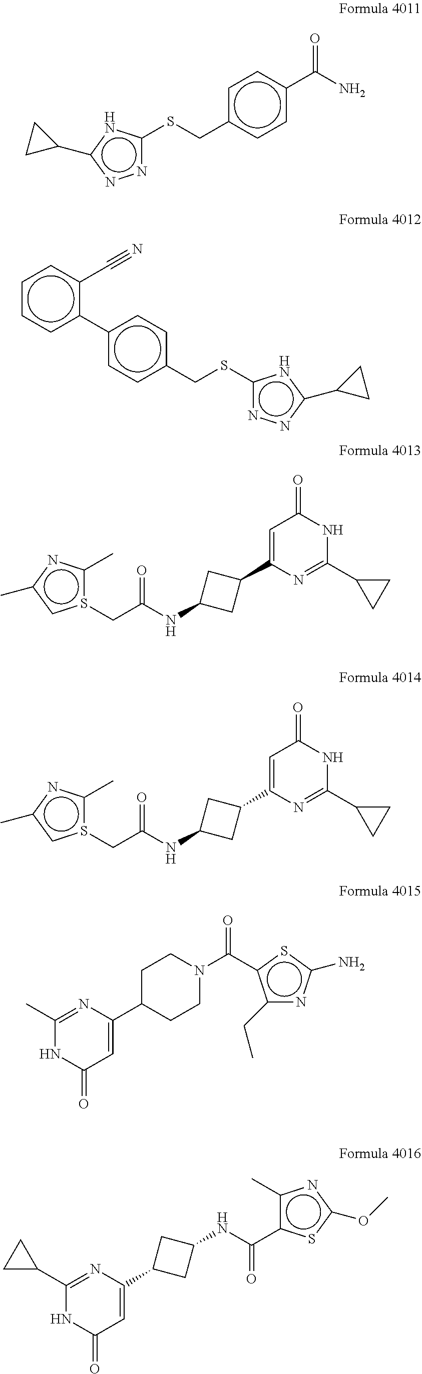

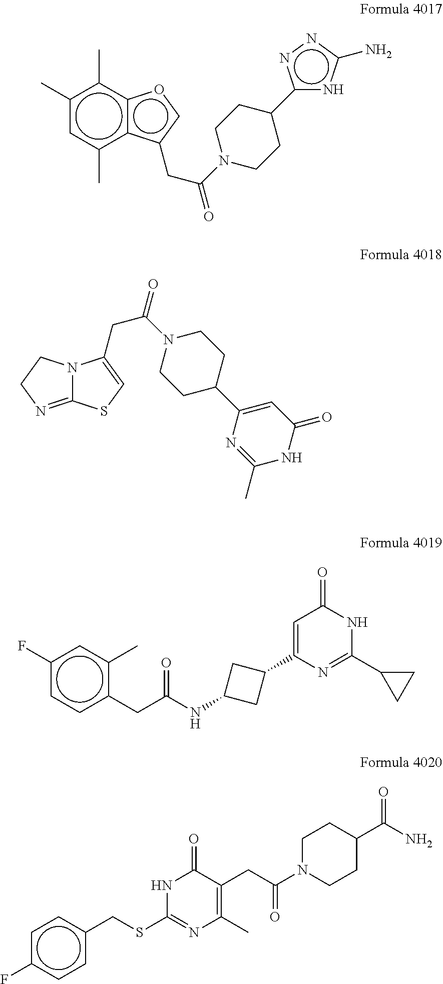

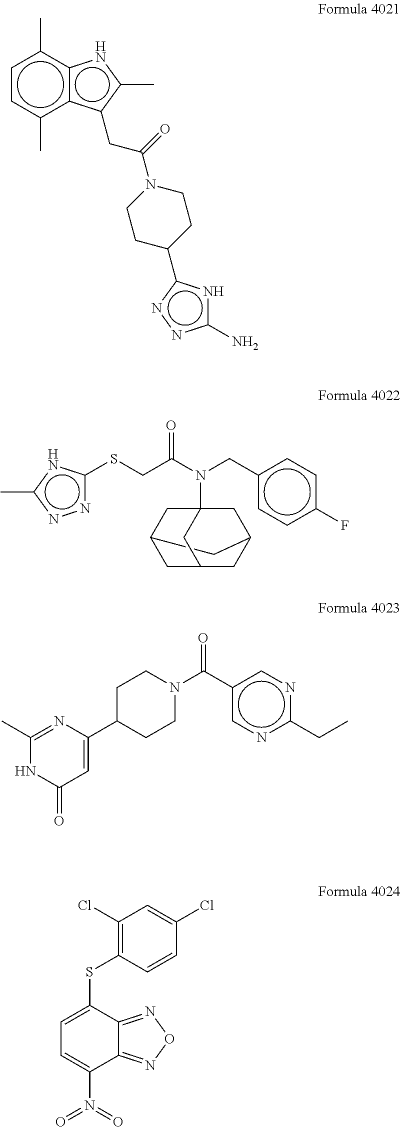

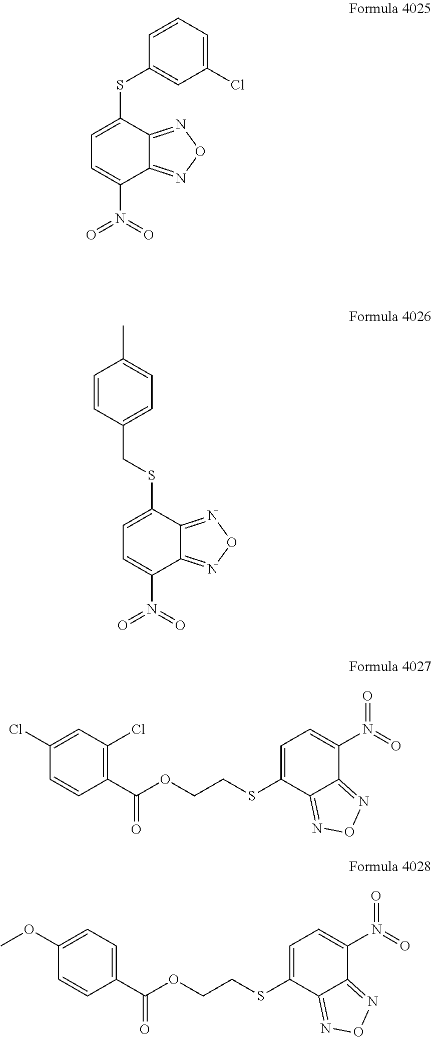 [SMILES: O=[N+]([O-])c1ccc(Sc2cccc(Cl)c2)c2nonc12 ; Cc1ccc(CSc2ccc([N+](=O)[O-])c3nonc23)cc1 ; O=C(OCCSc1ccc([N+](=O)[O-])c2nonc12)c1ccc(Cl)cc1Cl ; COc1ccc(C(=O)OCCSc2ccc([N+](=O)[O-])c3nonc23)cc1]

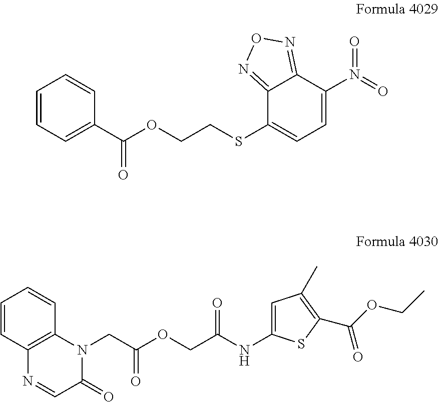 [SMILES: O=C(OCCSc1ccc([N+](=O)[O-])c2nonc12)c1ccccc1 ; CCOC(=O)c1sc(NC(=O)COC(=O)Cn2c(=O)cnc3ccccc32)cc1C]

11. The method of claim 1, wherein the compound has a formula selected from any one of Formulas 5001 to 5023:

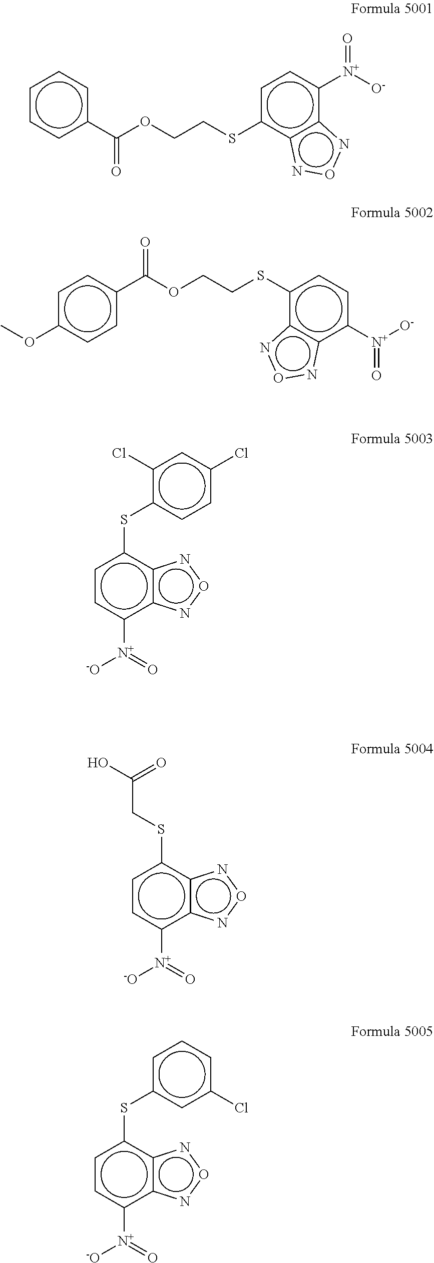

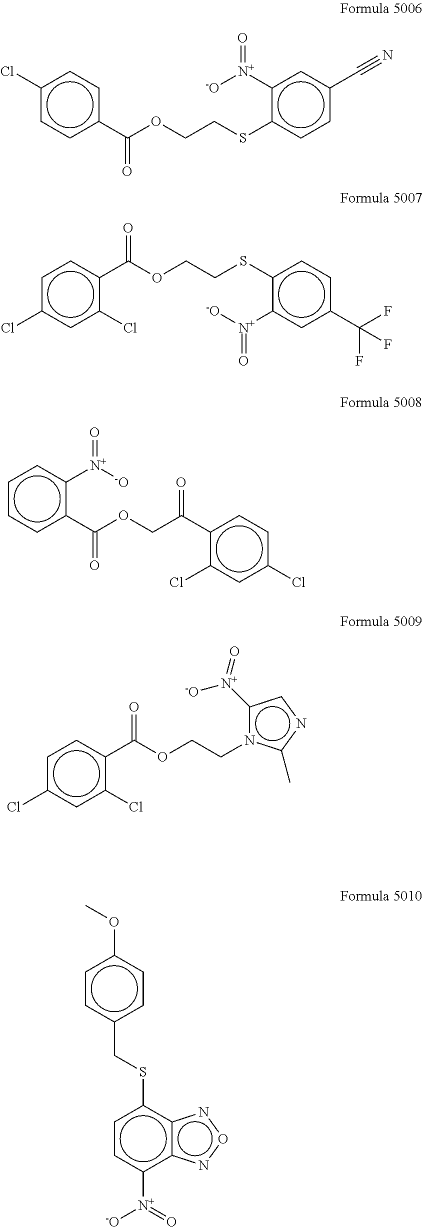

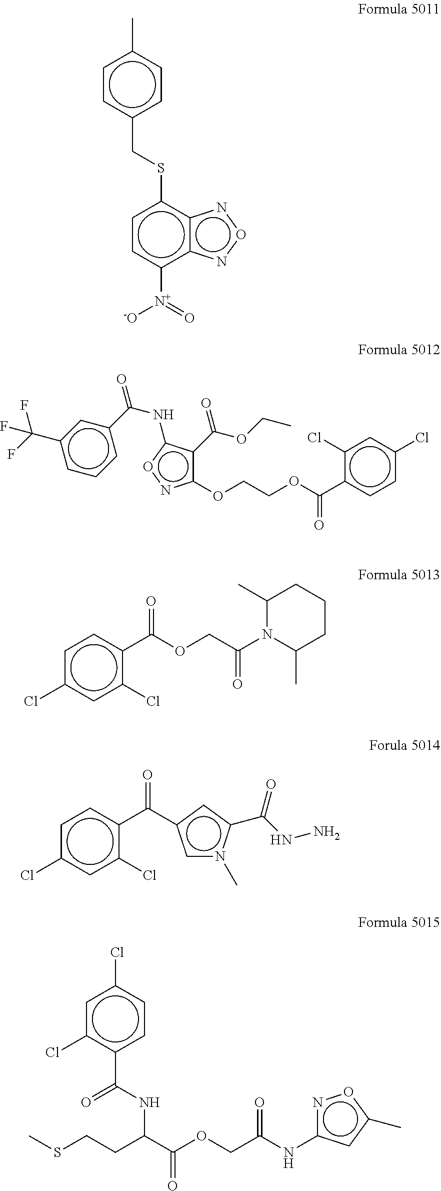

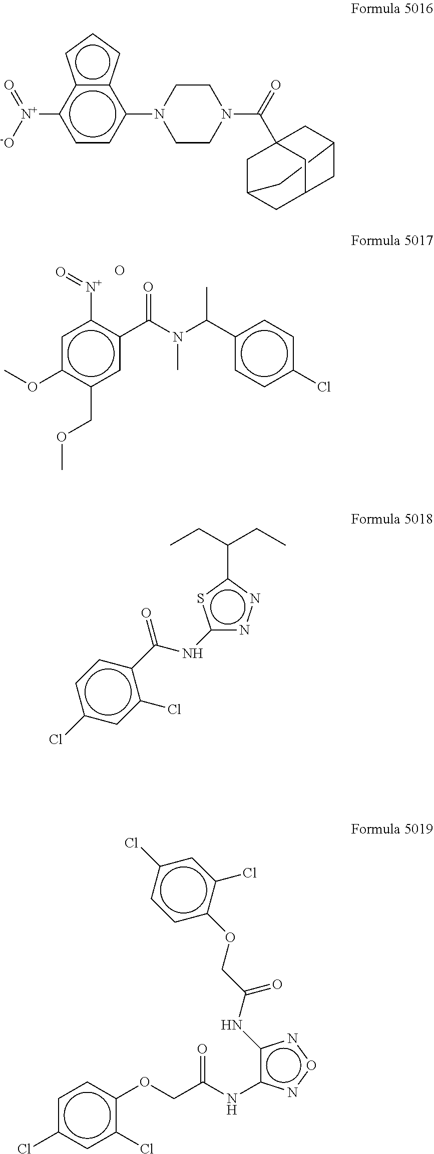

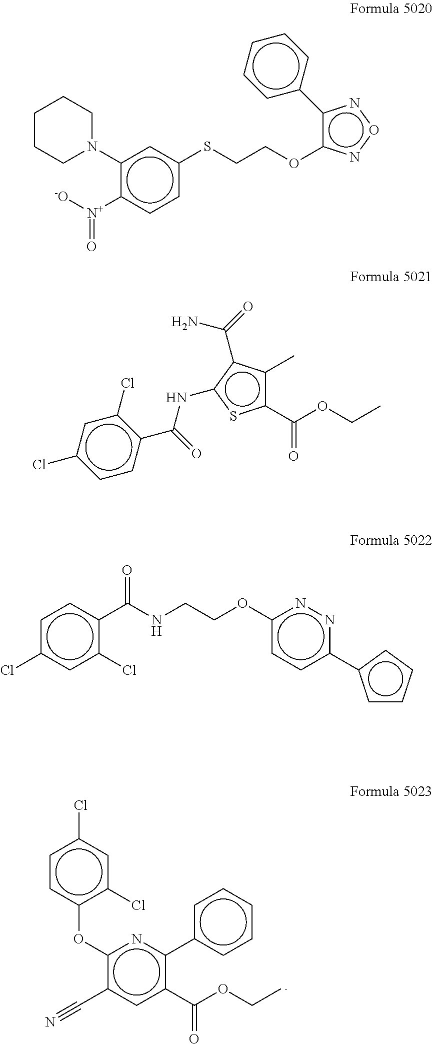

12. A method of treating or preventing a disease alleviated by inhibiting ANAT in a subject in need thereof, comprising administering to the subject a therapeutically effective amount of a compound of any one of Formulas VIII-a to VIII-e: 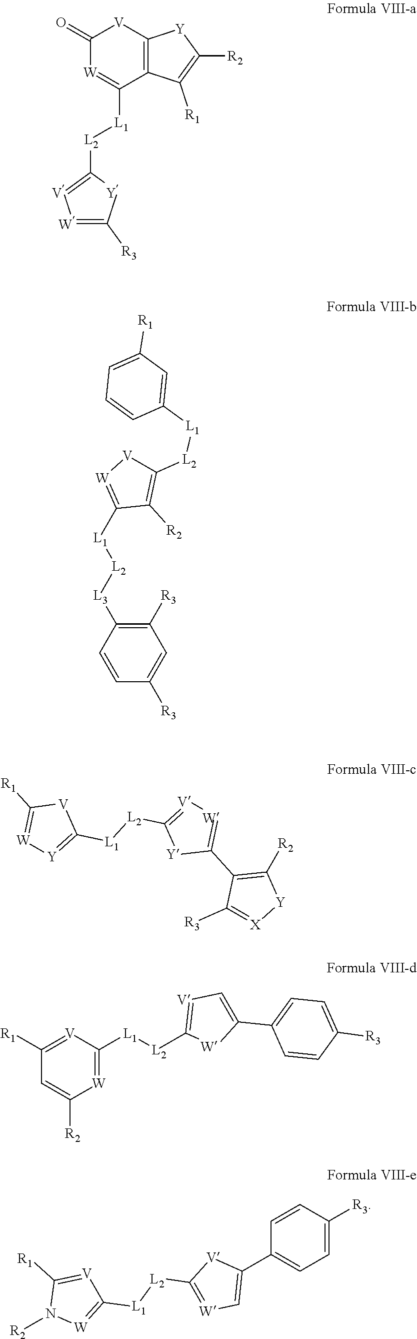
wherein in Formula VIII-a to VIII-e:
V, W, V′, W′, Y′, X, Y, and Z are independently selected at each occurrence from C, CH, CH₂, N, NH, NR^{a}, O, S
R₁, R₂, and R₃ are independently selected at each occurrence from hydrogen, optionally substituted alkylheteroaryl, optionally substituted alkyl, optionally substituted heteroalkyl, optionally substituted alkenyl, optionally substituted alkynyl, optionally substituted cycloalkyl, optionally substituted heterocycloalkyl, optionally substituted fused heterocycloalkyl, optionally substituted aryl, optionally substituted arylalkyl, optionally substituted heteroaryl, optionally substituted alkylheteroaryl, optionally substituted heteroarylalkyl, optionally substituted alkoxy, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂, wherein R₁, R₂, and R₃ can optionally be joined to form a carbo- or heterocycle;
L₁, L₂, and L₃ are linkers comprising independently at each occurrence one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a}C(O)—, —OC(O)O—, —OC(O)S—, —SC(O)O—, —OC(O)NR^{a}—, —NR^{a}C(O)O—, —S(O)ₜN(R^{a})— (where t is 1 or 2), —N(R^{a})S(O)ₜ— (where t is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl;
wherein any optional substituent is independently selected at each occurrence from optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, —SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), and PO₃(R^{a})₂; and
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.

13. The method of claim 12, wherein the compound has a formula selected from any one of Formulas 6001 to 6005:

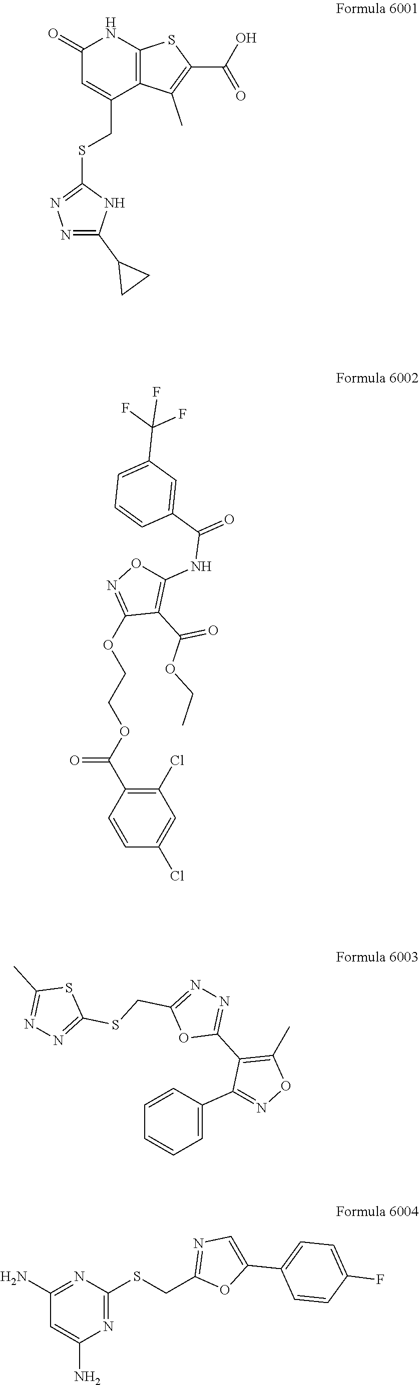

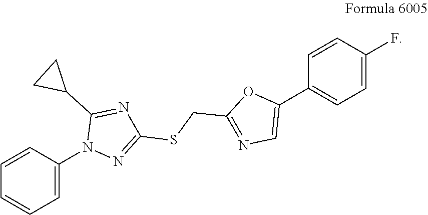 [SMILES: Fc1ccc(-c2cnc(CSc3nc(C4CC4)n(-c4ccccc4)n3)o2)cc1]

14. The method of claim 1 or claim 12, wherein the subject has a genetic defect affecting aspartoacylase enzyme activity.

15. The method of claim 1 or claim 12, wherein the subject has a deficient ability to metabolize N-acetylaspartate.

16. The method of claim 1 or claim 12, wherein the subject has elevated levels of the brain metabolite N-acetylaspartate (NAA).

17. The method of any one of claims 1 to 16, wherein the disease is Canavan disease.

18. The method of any one of claims 1 to 16, wherein the disease is cancer.

19. The method of any one of claims 1 to 16, wherein the disease is lung cancer.

20. The method of any one of claims 1 to 16, wherein the disease is an inborn error of metabolism.

21. The method of any one of claims 1 to 16, wherein the disease is epilepsy.

22. The method of any one of claims 1 to 16, wherein the disease is leukodystrophy.

23. The method of any one of claims 1 to 16, wherein the disease is N-acetyl aspartate deficiency.

24. The method of any one of claims 1 to 23, wherein the subject is human.

25. The method of any one of claims 1 to 24, wherein the compound is administered in a dosage unit form.

26. The method of claim 25, wherein the dosage unit comprises a physiologically compatible carrier medium.
